(19) United States
(12) Patent Application Publication
(10) Pub. No.: US 2024/0380463 A1
(43) Pub. Date: Nov. 14, 2024

(54) METHODS AND SYSTEMS FOR COMPRESSED CSI FOR VIRTUAL WIDEBAND CHANNELS

(71) Applicant: COGNITIVE SYSTEMS CORP., Waterloo (CA)

(72) Inventors: Chris BEG, Waterloo, Ontario (CA); Mohammad OMER, Waterloo, Ontario (CA)

(21) Appl. No.: 18/688,134

(22) PCT Filed: Sep. 2, 2022
(86) PCT No.: PCT/IB2022/058273

§ 371 Date: Feb. 29, 2024

Related U.S. Application Data
(60) Provisional application No. 63/271,328, filed on Oct. 25, 2021.
(60) Provisional application No. 63/240,645, filed on Sep. 3, 2021.

(51) Int. Cl. H04B 7/06; H04L 1/1607; H04L 27/26

(52) U.S. Cl. CPC H04B 7/0626; H04L 1/1614; H04L 27/2671

(57) ABSTRACT

Systems and methods for Wi-Fi sensing are provided. A method for Wi-Fi sensing carried out by a sensing receiver including a transmitting antenna, a receiving antenna, and a processor is described. Initially, sensing transmissions from a plurality of sensing transmitters are received. Then, a sensing measurement representing a channel state information (CSI) is generated based on the sensing transmissions. Thereafter, component frequency bands associated with a virtual wideband sensing transmission from a selected sensing transmitter of the plurality of sensing transmitters are identified. A reduced channel representation information (CRI) including the component frequency bands associated with the selected sensing transmitter is generated and component frequency bands associated with a remainder of the plurality of sensing transmitters are omitted. The reduced CRI is sent to a sensing algorithm manager.

### CROSS-REFERENCE TO RELATED APPLICATIONS

This application claims priority to U.S. Provisional Application No. 63/240,645, filed on Sep. 3, 2021, and U.S. Provisional Application No. 63/271,328, filed on Oct. 25, 2021, the entire contents of each of which are incorporated herein by reference.

### TECHNICAL FIELD

The present disclosure generally relates to systems and methods for Wi-Fi sensing. In particular, the present disclosure relates to systems and methods for compressed channel state information (CSI) for virtual wideband channels.

### BACKGROUND OF THE DISCLOSURE

Motion detection systems have been used to detect movement, for example, of objects in a room or an outdoor area. In some example motion detection systems, infrared or optical sensors are used to detect the movement of objects in the sensor's field of view. Motion detection systems have been used in security systems, automated control systems, and other types of systems. A Wi-Fi sensing system is one recent addition to motion detection systems. The Wi-Fi sensing system may be a network of Wi-Fi-enabled devices that may be a part of an IEEE 802.11 network. For example, the Wi-Fi sensing system may include a sensing receiver and a sensing transmitter. In an example, the Wi-Fi sensing system may be configured to detect features of interest in a sensing space. The sensing space may refer to any physical space in which the Wi-Fi sensing system may operate, such as a place of residence, a place of work, a shopping mall, a sports hall or sports stadium, a garden, or any other physical space. The features of interest may include motion of objects and motion tracking, presence detection, intrusion detection, gesture recognition, fall detection, breathing rate detection, and other applications.

In IEEE 802.11ac, and newer versions of the IEEE standard, channels may be formed by concatenating multiple contiguous component frequency bands. The concatenated multiple contiguous component frequency bands as a whole entity may be referred to as a wideband. For Wi-Fi sensing, using a wideband is advantageous as the wideband improves bandwidth efficiency and time resolution. There may be a scenario where sufficient contiguous component frequency bands may not be available to constitute a wideband even though there may be sufficient available component frequency bands. In an example, if available component frequency bands are not contiguous, then the available component frequency bands may not be available to constitute the wideband. Use of uplink orthogonal frequency division multiple access (UL-OFDMA) in the Wi-Fi sensing system currently enables contiguous component frequency bands to be concatenated for a same transmitting device (i.e., the sensing transmitter). An aggregated frequency band comprising multiple contiguous and/or non-contiguous component frequency bands is referred to as a virtual wideband.

In the Wi-Fi sensing system, information that is representative of a propagation channel (i.e., channel representation information) may need to be transmitted from one device to another device (for example, from the sensing receiver to the sensing transmitter) over the air. The representation of the propagation channel between devices is currently captured in channel state information (CSI). In case of the sensing transmitter which transmits a virtual wideband, isolated frequency bands between the non-contiguous component frequency bands may exist. Accordingly, when the sensing receiver calculates the CSI from the entire sensing transmission in the entire received band then it may combine together information from the sensing transmitter carried by the virtual wideband with other, unrelated information carried outside the virtual wideband. This calculated CSI may be distorted or rendered useless by this combination of information and so may not be included in the determination of movement or motion of an object.

### BRIEF SUMMARY OF THE DISCLOSURE

The present disclosure generally relates to systems and methods for Wi-Fi sensing. In particular, the present disclosure relates to systems and methods for compressed channel state information (CSI) for virtual wideband channels.

Systems and methods are provided for Wi-Fi sensing. In an example embodiment, a method for Wi-Fi sensing is described. The method is carried out by a sensing receiver including a transmitting antenna, a receiving antenna, and a processor configured to execute instructions. The method includes receiving sensing transmissions from a plurality of sensing transmitters, generating a sensing measurement representing a CSI based on the sensing transmissions, identifying component frequency bands associated with a virtual wideband sensing transmission from a selected sensing transmitter of the plurality of sensing transmitters, generating a reduced channel representation information (CRI) including the component frequency bands associated with the selected sensing transmitter and omitting component frequency bands associated with a remainder of the plurality of sensing transmitters, and sending the reduced CRI to a sensing algorithm manager.

In some embodiments, the component frequency bands associated with the sensing transmission are contiguous bands within a transmission channel.

In some embodiments, the component frequency bands associated with the sensing transmission include non-contiguous bands within a transmission channel.

In some embodiments, generating the reduced CRI includes generating a full time-domain channel representation information (TD-CRI) of the CSI, generating a reduced TD-CRI including time domain representations of the component frequency bands associated with the selected sensing transmitter and omitting component frequency bands associated with a remainder of the plurality of sensing transmitters, and generating a frequency domain bit map indicating locations of the time domain representations in the full TD-CRI.

In some embodiments, the method further includes generating a reduced filtered TD-CRI including principal impulses of the reduced TD-CRI, where the principal impulses represent a subset of time domain pulses of the full TD-CRI and generating location information indicating locations of the principal impulses in the reduced TD-CRI.

In some embodiments, the principal impulses are selected to permit reconstruction of the reduced TD-CRI.

In some embodiments, the location information includes a bit map.

In some embodiments, the location information is included in the reduced filtered TD-CRI.

In some embodiments, the method further includes obtaining, by the sensing algorithm manager, the reduced CRI, generating, by the sensing algorithm manager, a reconstructed CSI based on the reduced CRI, and executing, by the sensing algorithm manager, a sensing algorithm according to the reconstructed CSI to obtain a sensing result.

In some embodiments, the method further includes obtaining, by the sensing algorithm manager, the reduced filtered TD-CRI, the location information, and the frequency domain bit map, generating, by the sensing algorithm manager, a reconstructed TD-CRI based on the location information, the frequency domain bit map, and the principal impulses of the reduced filtered TD-CRI, generating, by the sensing algorithm manager, a reconstructed CSI according to the reconstructed TD-CRI, and executing, by the sensing algorithm manager, a sensing algorithm according to the reconstructed CSI to obtain a sensing result.

In another example embodiment, a method for Wi-Fi sensing is described. The method is carried out by a device including a receiving antenna and a processor configured to execute instructions. The method includes receiving, via the receiving antenna, a reduced channel representation information (CRI) including component frequency bands associated with a selected sensing transmitter from a plurality of sensing transmitters and omitting component frequency bands associated with a remainder of the plurality of sensing transmitters, generating, by a sensing algorithm manager operating on the processor, a reconstructed time-domain channel representation information (TD-CRI) from the reduced CRI, transforming the reconstructed TD-CRI into a reconstructed CSI, and executing, by the sensing algorithm manager, a sensing algorithm on the reconstructed CSI to obtain a sensing result.

In some embodiments, the reduced CRI is a reduced TD-CRI including time domain representations of the component frequency bands associated with the selected sensing transmitter and omitting component frequency bands associated with a remainder of the plurality of sensing transmitters, and a frequency domain bit map indicating locations of the time domain representations in a full TD-CRI.

In some embodiments, the reduced CRI is a reduced filtered TD-CRI including time domain representations of principal impulses of the component frequency bands associated with the selected sensing transmitter and omitting component frequency bands associated with a remainder of the plurality of sensing transmitters, a frequency domain bit map indicating the locations of the time domain representations in a full TD-CRI, and location information indicating the locations of the principal impulses in the full TD-CRI.

In another example embodiment, a system for Wi-Fi sensing is described. The system including a sensing receiver having a transmitting antenna, a receiving antenna, and at least one processor configured to execute instructions for receiving, via the receiving antenna, sensing transmissions from a plurality of sensing transmitters; generating a sensing measurement representing a channel state information (CSI) based on the sensing transmissions; identifying component frequency bands associated with a virtual wideband sensing transmission from a selected sensing transmitter of the plurality of sensing transmitters; generating a reduced channel representation information (CRI) including the component frequency bands associated with the selected sensing transmitter and omitting component frequency bands associated with a remainder of the plurality of sensing transmitters; and sending the reduced CRI to a sensing algorithm manager.

In some embodiments, the component frequency bands associated with the sensing transmission are contiguous bands within a transmission channel.

In some embodiments, the component frequency bands associated with the sensing transmission include non-contiguous bands within a transmission channel.

In some embodiments, generating the reduced CRI includes generating a full time-domain channel representation information (TD-CRI) of the CSI; generating a reduced TD-CRI including time domain representations of the component frequency bands associated with the selected sensing transmitter and omitting component frequency bands associated with a remainder of the plurality of sensing transmitters; and generating a frequency domain bit map indicating the-locations of the time domain representations in the full TD-CRI.

In some embodiments, the at least one processor is further configured with instructions for generating a reduced filtered TD-CRI including principal impulses of the reduced TD-CRI, the principal impulses representing a subset of time domain pulses of the full TD-CRI; and generating location information indicating locations of the principal impulses in the reduced TD-CRI.

In some embodiments, the principal impulses are selected to permit reconstruction of the reduced TD-CRI.

In some embodiments, the location information includes a bit map.

In some embodiments, the location information is included in the reduced filtered TD-CRI.

In some embodiments, the at least one processor is further configured with instructions for obtaining, by the sensing algorithm manager, the reduced CRI; generating, by the sensing algorithm manager, a reconstructed CSI based on the reduced CRI; and executing, by the sensing algorithm manager, a sensing algorithm according to the reconstructed CSI to obtain a sensing result.

In some embodiments, the system further includes obtaining, by the sensing algorithm manager, the reduced filtered TD-CRI, the location information, and the frequency domain bit map; generating, by the sensing algorithm manager, a reconstructed TD-CRI based on the location information, the frequency domain bit map, and the principal impulses of the filtered TD-CRI; generating, by the sensing algorithm manager, a reconstructed CSI according to the reconstructed TD-CRI; and executing, by the sensing algorithm manager, a sensing algorithm according to the reconstructed CSI to obtain a sensing result.

In another example embodiment, a system for Wi-Fi sensing is described. The system includes a sensing receiver having a transmitting antenna, a receiving antenna, and at least one processor configured to execute instructions for receiving a reduced channel representation information (CRI) including component frequency bands associated with a selected sensing transmitter from a plurality of sensing transmitters and omitting component frequency bands associated with a remainder of the plurality of sensing transmitters; generating, by a sensing algorithm manager, a reconstructed time-domain channel representation information (TD-CRI) from the reduced CRI; transforming the reconstructed TD-CRI into a reconstructed channel state information (CSI); and executing, by the sensing algorithm manager, a sensing algorithm on the reconstructed CSI to obtain a sensing result.

In some embodiments, the reduced CRI is a reduced TD-CRI including time domain representations of the component frequency bands associated with the selected sensing transmitter and omitting component frequency bands associated with a remainder of the plurality of sensing transmitters, and a frequency domain bit map indicating the locations of the time domain representations in a full TD-CRI.

In some embodiments, the reduced CRI is a reduced filtered TD-CRI including time domain representations of principal impulses of the component frequency bands associated with the selected sensing transmitter and omitting component frequency bands associated with a remainder of the plurality of sensing transmitters, a frequency domain bit map indicating the locations of the time domain representations in a full TD-CRI, and location information indicating the locations of the principal impulses in the full TD-CRI.

Other aspects and advantages of the disclosure will become apparent from the following detailed description, taken in conjunction with the accompanying drawings, which illustrate by way of example, the principles of the disclosure.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a diagram showing an example wireless communication system;

FIG. 2A and FIG. 2B are diagrams showing example wireless signals communicated between wireless communication devices;

FIG. 3A and FIG. 3B are plots showing examples of channel responses computed from the wireless signals communicated between wireless communication devices in FIG. 2A and FIG. 2B;

FIG. 4A and FIG. 4B are diagrams showing example channel responses associated with motion of an object in distinct regions of a space;

FIG. 4C and FIG. 4D are plots showing the example channel responses of FIG. 4A and FIG. 4B overlaid on an example channel response associated with no motion occurring in the space;

FIG. 5 depicts an implementation of some of an architecture of an implementation of a system for Wi-Fi sensing, according to some embodiments;

FIG. 6 depicts an uplink orthogonal frequency division multiple access (UL-OFDMA) transmission procedure and a format of a trigger frame, according to some embodiments;

FIG. 7A to FIG. 7H depict a hierarchy of fields within the trigger frame, according to some embodiments;

FIG. 8 depicts a sequence diagram for communication between a sensing receiver, a sensing transmitter, and a sensing algorithm manager, where the sensing receiver is the sensing initiator, according to some embodiments;

FIG. 9 depicts a sequence diagram for communication between a sensing receiver, a sensing transmitter, and a sensing algorithm manager, where the sensing transmitter is a sensing initiator, according to some embodiments;

FIG. 10 illustrates an example of a component of a management frame carrying a channel representation information (CRI) transmission message, according to some embodiments;

FIG. 11A and FIG. 11B depict four 20 MHz component frequency bands and their availability status, according to some embodiments;

FIG. 12 depicts an example of a virtual wideband for a multi-user sensing transmission, according to some embodiments;

FIG. 13 illustrates a representation of a receiver chain of a sensing receiver, according to some embodiments;

FIG. 14 depicts an example representation of a channel, which includes a direct signal path and a single multipath, according to some embodiments;

FIG. 15 depicts creation of a constrained basis matrix, according to some embodiments;

FIG. 16 depicts an example of a virtual wideband of a sensing transmitter, according to some embodiments;

FIG. 17 depicts an UL-OFDMA signal received by a sensing receiver, according to some embodiments;

FIG. 18 illustrates a representation of communication of locations of principal impulses from a sensing receiver to a sensing algorithm manager using an active tone bit map, according to some embodiments;

FIG. 19 illustrates a representation of communication of locations of principal impulses from a sensing receiver to a sensing algorithm manager using a full bit map, according to some embodiments;

FIG. 20 illustrates a representation of communication of locations of principal impulses from a sensing receiver to a sensing algorithm manager using positions of the principal impulses in a full time-domain channel representation (TD-CRI), according to some embodiments;

FIG. 21A and FIG. 21B depict a flowchart for sending a reduced channel representation information (CRI) to a sensing algorithm manager, according to some embodiments; and

FIG. 22 depicts a flowchart for executing a sensing algorithm on a reconstructed channel state information (CSI) to obtain a sensing result, according to some embodiments.

### DETAILED DESCRIPTION

In some aspects of what is described herein, a wireless sensing system may be used for a variety of wireless sensing applications by processing wireless signals (e.g., radio frequency signals) transmitted through a space between wireless communication devices. Example wireless sensing applications include motion detection, which can include the following: detecting motion of objects in the space, motion tracking, breathing detection, breathing monitoring, presence detection, gesture detection, gesture recognition, human detection (moving and stationary human detection), human tracking, fall detection, speed estimation, intrusion detection, walking detection, step counting, respiration rate detection, apnea estimation, posture change detection, activity recognition, gait rate classification, gesture decoding, sign language recognition, hand tracking, heart rate estimation, breathing rate estimation, room occupancy detection, human dynamics monitoring, and other types of motion detection applications. Other examples of wireless sensing applications include object recognition, speaking recognition, keystroke detection and recognition, tamper detection, touch detection, attack detection, user authentication, driver fatigue detection, traffic monitoring, smoking detection, school violence detection, human counting, metal detection, human recognition, bike localization, human queue estimation, Wi-Fi imaging, and other types of wireless sensing applications. For instance, the wireless sensing system may operate as a motion detection system to detect the existence and location of motion based on Wi-Fi signals or other types of wireless signals. As described in more detail below, a wireless sensing system may be configured to control measurement rates, wireless connections, and device participation, for example, to improve system operation or to achieve other technical advantages. The system improvements and technical advantages achieved when the wireless sensing system is used for motion detection are also achieved in examples where the wireless sensing system is used for another type of wireless sensing application.

In some example wireless sensing systems, a wireless signal includes a component (e.g., a synchronization preamble in a Wi-Fi PHY frame, or another type of component) that wireless devices can use to estimate a channel response or other channel information, and the wireless sensing system can detect motion (or another characteristic depending on the wireless sensing application) by analyzing changes in the channel information collected over time. In some examples, a wireless sensing system can operate similar to a bistatic radar system, where a Wi-Fi access point (AP) assumes the receiver role, and each Wi-Fi device (stations or nodes or peers) connected to the AP assumes the transmitter role. The wireless sensing system may trigger a connected device to generate a transmission and produce a channel response measurement at a receiver device. This triggering process can be repeated periodically to obtain a sequence of time variant measurements. A wireless sensing algorithm may then receive the generated time-series of channel response measurements (e.g., computed by Wi-Fi receivers) as input, and through a correlation or filtering process, may then make a determination (e.g., determine if there is motion or no motion within the environment represented by the channel response, for example, based on changes or patterns in the channel estimations). In examples where the wireless sensing system detects motion, it may also be possible to identify a location of the motion within the environment based on motion detection results among a number of wireless devices.

Accordingly, wireless signals received at each of the wireless communication devices in a wireless communication network may be analyzed to determine channel information for the various communication links (between respective pairs of wireless communication devices) in the network. The channel information may be representative of a physical medium that applies a transfer function to wireless signals that traverse a space. In some instances, the channel information includes a channel response. Channel responses can characterize a physical communication path, representing the combined effect of, for example, scattering, fading, and power decay within the space between the transmitter and receiver. In some instances, the channel information includes beamforming state information (e.g., a feedback matrix, a steering matrix, channel state information (CSI), etc.) provided by a beamforming system. Beamforming is a signal processing technique often used in multi-antenna (multiple-input/multiple-output (MIMO)) radio systems for directional signal transmission or reception. Beamforming can be achieved by operating elements in an antenna array in such a way that signals at some angles experience constructive interference while others experience destructive interference.

The channel information for each of the communication links may be analyzed (e.g., by a hub device or other device in a wireless communication network, or a sensing transmitter communicably coupled to the network) to, for example, detect whether motion has occurred in the space, to determine a relative location of the detected motion, or both. In some aspects, the channel information for each of the communication links may be analyzed to detect whether an object is present or absent, e.g., when no motion is detected in the space.

In some cases, a wireless sensing system can control a node measurement rate. For instance, a Wi-Fi motion system may configure variable measurement rates (e.g., channel estimation/environment measurement/sampling rates) based on criteria given by a current wireless sensing application (e.g., motion detection). In some implementations, when no motion is present or detected for a period of time, for example, the wireless sensing system can reduce the rate that the environment is measured, such that the connected device will be triggered less frequently. In some implementations, when motion is present, for example, the wireless sensing system can increase the triggering rate to produce a time-series of measurements with finer time resolution. Controlling the variable measurement rate can allow energy conservation (through the device triggering), reduce processing (less data to correlate or filter), and improve resolution during specified times.

In some cases, a wireless sensing system can perform band steering or client steering of nodes throughout a wireless network, for example, in a Wi-Fi multi-AP or Extended Service Set (ESS) topology, multiple coordinating wireless APs each provide a Basic Service Set (BSS) which may occupy different frequency bands and allow devices to transparently move between from one participating AP to another (e.g., mesh). For instance, within a home mesh network, Wi-Fi devices can connect to any of the APs, but typically select one with a good signal strength. The coverage footprint of the mesh APs typically overlap, often putting each device within communication range or more than one AP. If the AP supports multi-bands (e.g., 2.4 GHz and 5 GHZ), the wireless sensing system may keep a device connected to the same physical AP, but instruct it to use a different frequency band to obtain more diverse information to help improve the accuracy or results of the wireless sensing algorithm (e.g., motion detection algorithm). In some implementations, the wireless sensing system can change a device from being connected to one mesh AP to being connected to another mesh AP. Such device steering can be performed, for example, during wireless sensing (e.g., motion detection), based on criteria detected in a specific area, to improve detection coverage, or to better localize motion within an area.

In some cases, a wireless sensing system can allow devices to dynamically indicate and communicate their wireless sensing capability or wireless sensing willingness to the wireless sensing system. For example, there may be times when a device does not want to be periodically interrupted or triggered to transmit a wireless signal that would allow the AP to produce a channel measurement. For instance, if a device is sleeping, frequently waking the device up to transmit or receive wireless sensing signals could consume resources (e.g., causing a cell-phone battery to discharge faster). These and other events could make a device willing or not willing to participate in wireless sensing system operations. In some cases, a cell phone running on its battery may not want to participate, but when the cell phone is plugged into the charger, it may be willing to participate. Accordingly, if the cell phone is unplugged, it may indicate to the wireless sensing system to exclude the cell phone from participating; whereas if the cell phone is plugged in, it may indicate to the wireless sensing system to include the cell phone in wireless sensing system operations. In some cases, if a device is under load (e.g., a device streaming audio or video) or busy performing a primary function, the device may not want to participate; whereas when the same device's load is reduced and participating will not interfere with a primary function, the device may indicate to the wireless sensing system that it is willing to participate.

Example wireless sensing systems are described below in the context of motion detection (detecting motion of objects in the space, motion tracking, breathing detection, breathing monitoring, presence detection, gesture detection, gesture recognition, human detection (moving and stationary human detection), human tracking, fall detection, speed estimation, intrusion detection, walking detection, step counting, respiration rate detection, apnea estimation, posture change detection, activity recognition, gait rate classification, gesture decoding, sign language recognition, hand tracking, heart rate estimation, breathing rate estimation, room occupancy detection, human dynamics monitoring, and other types of motion detection applications. However, the operation, system improvements, and technical advantages achieved when the wireless sensing system is operating as a motion detection system are also applicable in examples where the wireless sensing system is used for another type of wireless sensing application.

In various embodiments of the disclosure, non-limiting definitions of one or more terms that will be used in the document are provided below.

A term “measurement campaign” may refer to a bi-directional series of one or more sensing transmissions between a sensing receiver and a sensing transmitter that allows a series of one or more sensing measurements to be computed.

A term “channel state information (CSI)” may refer to properties of a communications channel that are known or measured by a technique of channel estimation. CSI may represent how wireless signals propagate from a transmitter (for example, a sensing transmitter) to a receiver (for example, a sensing receiver) along multiple paths. CSI is typically a matrix of complex values representing the amplitude attenuation and phase shift of signals, which provides an estimation of a communications channel.

A term “full time-domain channel representation information (full TD-CRI)” may refer to a series of complex pairs representing the amplitude and delay of time domain pulses which are created by performing an inverse discrete Fourier transform (IDFT) on CSI values, for example CSI calculated by a baseband receiver.

A term “filtered time-domain channel representation information (filtered TD-CRI)” may refer to a reduced series of complex pairs of time domain pulses created by applying an algorithm to a full TD-CRI. The algorithm may select some time domain pulses and reject others. The filtered TD-CRI includes information that relates a selected time domain pulse to the corresponding time domain pulse in the full TD-CRI.

A term “reduced filtered TD-CRI” may refer to a reduced series of complex pairs of time domain pulses created by applying an algorithm to a filtered TD-CRI. The algorithm may select some time domain pulses and reject others. An example of the reduced filtered TD-CRI is reduced CRI. The reduced CRI is a reduced filtered TD-CRI including time domain representations of principal impulses of the component frequency bands associated with a sensing transmitter

A term “principal impulses” may refer to a minimum subset of TD-CRI time domain pulses comprising the time domain pulses which are determined to be principal for creating reconstructed CSI (R-CSI) channel representation with sufficient accuracy. In an example, principal impulses are included in the filtered TD-CRI.

A term “channel representation information (CRI)” may refer to a collection of sensing measurements which together represent the state of the channel between two devices. Examples of CRI are CSI and full TD-CRI.

A term “reconstructed CSI (R-CSI)” may refer to a representation of original CSI values as measured by the baseband receiver, where R-CSI is calculated by taking original CSI values (frequency domain), performing an IDFT to translate those values into the time domain, selecting a number of time domain pulses, zeroing or nulling time domain tones that do not include a selected time domain pulse, and performing a DFT. The resulting frequency domain complex values are the R-CSI.

A term “discrete Fourier transform (DFT)” may refer to an algorithm that transforms a signal in time domain to a signal in frequency domain. In an example, the DFT may be used to transform a TD-CRI into a R-CSI. In an embodiment, a fast Fourier transform (FFT) may be used to implement the DFT.

A term “fast Fourier transform (FFT)” may refer to a fast algorithm to implement DFT.

A term “inverse discrete Fourier transform (IDFT)” may refer to an algorithm which transforms a signal in frequency domain to a signal in time domain. In an example, the IDFT may be used to transform a CSI into a TD-CRI. In an embodiment, an inverse fast Fourier transform (IFFT) may be used to implement the IDFT.

A term “inverse fast Fourier transform (IFFT)” may refer to a fast algorithm to implement IDFT.

A term “sensing initiator” may refer to a device that initiates a Wi-Fi sensing session. The role of sensing initiator may be taken on by the sensing receiver, the sensing transmitter, or a separate device which includes a sensing algorithm (for example, a sensing algorithm manager).

A term “Null Data PPDU (NDP)” may refer to a PPDU that does not include data fields. In an example, Null Data PPDU may be used for sensing transmission where it is the MAC header that includes the information required.

A term “sensing transmission” may refer to any transmission made from the sensing transmitter to the sensing receiver which may be used to make a sensing measurement. In an example, sensing transmission may also be referred to as wireless sensing signal or wireless signal.

A term “resource unit (RU)” may refer to an allocation of orthogonal frequency division multiplexing (OFDM) channels which may be used to carry a modulated signal. An RU may include a variable number of carriers depending on the mode of the modem.

A term “sensing trigger message” may refer to a message sent from the sensing receiver to the sensing transmitter to trigger one or more sensing transmissions that may be used for performing sensing measurements. A sensing trigger message may also be referred to as a sensing initiation message.

A term “uplink orthogonal frequency division multiple access (UL-OFDMA) sensing trigger message” may refer to a message from the sensing receiver to one or more sensing transmitters to generate a sensing transmission in a single transmission opportunity (TXOP) using UL-OFDMA. The UL-OFMDA sensing trigger message includes data which instructs the one or more sensing transmitters how to form sensing transmissions in response to the UL-OFMDA sensing trigger message.

A term “sensing response message” may refer to a message which is included within a sensing transmission from the sensing transmitter to the sensing receiver. In an example, the sensing transmission that includes the sensing response message may be used to perform a sensing measurement.

A term “sensing measurement” may refer to a measurement of a state of a channel i.e., CSI measurement between the sensing transmitter and the sensing receiver derived from a sensing transmission.

A term “PHY-layer Protocol Data Unit (PPDU)” may refer to a data unit that includes preamble and data fields. The preamble field may include the transmission vector format information and the data field may include payload and higher layer headers.

A term “sensing transmitter” may refer to a device that sends a transmission (for example, NDP and PPDUs) used for sensing measurements (for example, channel state information) in a sensing session. In an example, a station is an example of a sensing transmitter. In some examples, an access point (AP) may also be a sensing transmitter for Wi-Fi sensing purposes in the example where a station acts as a sensing receiver.

A term “sensing receiver” may refer to a device that receives a transmission (for example, NDP and PPDUs) sent by a sensing transmitter and performs one or more sensing measurements (for example, channel state information) in a sensing session. An access point (AP) is an example of a sensing receiver. In some examples, a station may also be a sensing receiver in a mesh network scenario.

A term “sensing transmission announcement message” may refer to a message which is sent from the sensing transmitter to the sensing receiver that announces that a sensing transmission NDP will follow within a short interframe space (SIFS). The sensing transmission NDP may be transmitted using transmission parameters defined with the sensing transmission announcement messages.

A term “short interframe space (SIFS)” may refer to a period within which a processing element (for example, a microprocessor, dedicated hardware, or any such element) within a device of a Wi-Fi sensing system is able to process data presented to it in a frame. In an example, the short interframe space may be 10 μs.

A term “sensing transmission NDP” may refer to an NDP transmission which is sent by the sensing transmitter and used for a sensing measurement at the sensing receiver. The transmission follows a sensing transmission announcement and may be transmitted using transmission parameters that are defined in the sensing response announcement.

A term “transmission opportunity (TXOP)” may refer to a negotiated interval of time during which a particular quality of service (QOS) station (e.g., a sensing initiator or sensing transmitter) may have the right to initiate a frame exchange onto a wireless medium. A QoS access category (AC) of the transmission opportunity may be requested as part of a negotiation.

A term “quality of service (QOS) access category (AC)” may refer to an identifier for a frame which classifies a priority of transmission that the frame requires. In an example, four QoS access categories are defined namely AC_VI: Video, AC_VO: Voice, AC_BE: Best-Effort, and AC_BK: Background. Further, each QoS access category may have differing transmission opportunity parameters defined for it.

A term “transmission parameters” may refer to a set of IEEE 802.11 PHY transmitter configuration parameters which are defined as part of transmission vector (TXVECTOR) corresponding to a specific PHY and which are configurable for each PHY-layer Protocol Data Unit (PPDU) transmission.

A term “channel response information (CRI) transmission message” may refer to a message sent by the sensing receiver that has performed a sensing measurement on a sensing transmission, in which the sensing receiver sends CRI to the sensing transmitter.

A term “time domain pulse” may refer to a complex number that represents amplitude and phase of discretized energy in the time domain. When CSI values are obtained for each tone from the baseband receiver, time domain pulses are obtained by performing an IFFT on the CSI values.

A term “tone” may refer to an individual subcarrier in an OFDM signal. A tone may be represented in time domain or frequency domain. In the time domain, a tone may also be referred to as a symbol. In the frequency domain, a tone may also be referred to as a subcarrier.

A term “delivered transmission configuration” may refer to transmission parameters applied by the sensing transmitter to a sensing transmission.

A term “requested transmission configuration” may refer to requested transmission parameters of the sensing transmitter to be used when sending a sensing transmission.

A term “virtual wideband” may refer to an aggregated frequency band comprising multiple contiguous and/or non-contiguous component frequency bands.

A term “Wi-Fi sensing session” may refer to a period during which objects in a physical space may be probed, detected and/or characterized. In an example, during a Wi-Fi sensing session, several devices participate in, and thereby contribute to the generation of sensing measurements. A Wi-Fi sensing session may also be referred to as a wireless local area network (WLAN) sensing session or simply a sensing session.

A term “steering matrix configuration” may refer to a matrix of complex values representing real and complex phase required to pre-condition antenna of a Radio Frequency (RF) transmission signal chain for each transmit signal. Application of the steering matrix configuration (for example, by a spatial mapper) enables beamforming and beam-steering.

A term “spatial mapper” may refer to a signal processing element that adjusts the amplitude and phase of a signal input to an RF transmission chain in a station or a sensing transmitter. The spatial mapper may include elements to process the signal to each RF chain implemented. The operation carried out is called spatial mapping. The output of the spatial mapper is one or more spatial streams.

For purposes of reading the description of the various embodiments below, the following descriptions of the sections of the specifications and their respective contents may be helpful:

Section A describes a wireless communications system, wireless transmissions and sensing measurements which may be useful for practicing embodiments described herein.

Section B describes systems and methods that are useful for a Wi-Fi sensing system configurated to send sensing transmissions and make sensing measurements.

Section C describes embodiments of systems and methods for compressed CSI for virtual wideband channels.

### A. Wireless Communications System, Wireless Transmissions, and Sensing Measurements

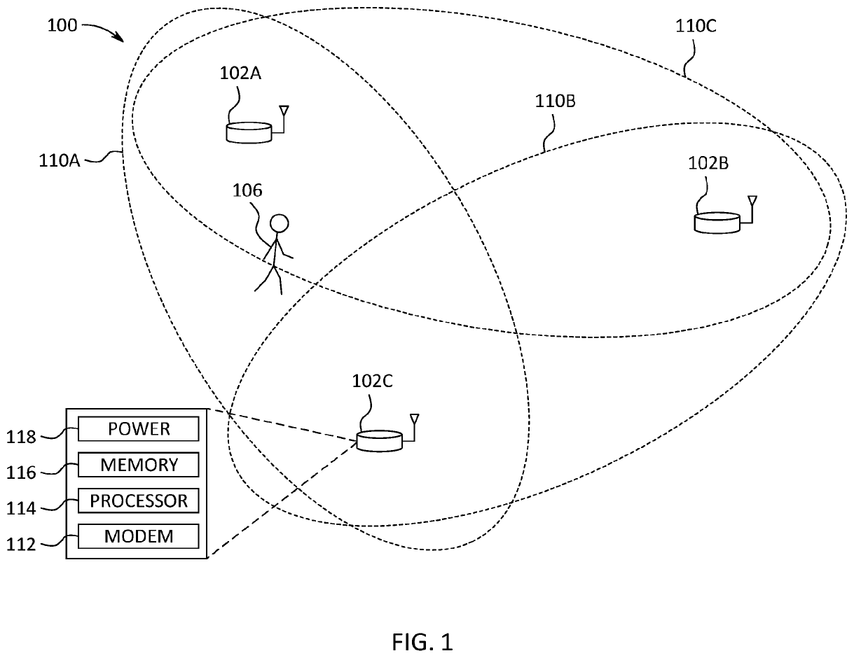

FIG. 1 illustrates wireless communication system 100. Wireless communication system 100 includes three wireless communication devices: first wireless communication device 102A, second wireless communication device 102B, and third wireless communication device 102C. Wireless communication system 100 may include additional wireless communication devices and other components (e.g., additional wireless communication devices, one or more network servers, network routers, network switches, cables, or other communication links, etc.).

Wireless communication devices 102A, 102B, 102C can operate in a wireless network, for example, according to a wireless network standard or another type of wireless communication protocol. For example, the wireless network may be configured to operate as a Wireless Local Area Network (WLAN), a Personal Area Network (PAN), a metropolitan area network (MAN), or another type of wireless network. Examples of WLANs include networks configured to operate according to one or more of the 802.11 family of standards developed by IEEE (e.g., Wi-Fi networks), and others. Examples of PANs include networks that operate according to short-range communication standards (e.g., BLUETOOTH®., Near Field Communication (NFC), ZigBee), millimeter wave communications, and others.

In some implementations, wireless communication devices 102A, 102B, 102C may be configured to communicate in a cellular network, for example, according to a cellular network standard. Examples of cellular networks include networks configured according to 2G standards such as Global System for Mobile (GSM) and Enhanced Data rates for GSM Evolution (EDGE) or EGPRS; 3G standards such as Code Division Multiple Access (CDMA), Wideband Code Division Multiple Access (WCDMA), Universal Mobile Telecommunications System (UMTS), and Time Division Synchronous Code Division Multiple Access (TD-SCDMA); 4G standards such as Long-Term Evolution (LTE) and LTE-Advanced (LTE-A); 5G standards, and others.

In the example shown in FIG. 1, wireless communication devices 102A, 102B, 102C can be, or they may include standard wireless network components. For example, wireless communication devices 102A, 102B, 102C may be commercially available Wi-Fi APs or another type of wireless access point (WAP) performing one or more operations as described herein that are embedded as instructions (e.g., software or firmware) on the modem of the WAP. In some cases, wireless communication devices 102A, 102B, 102C may be nodes of a wireless mesh network, such as, for example, a commercially available mesh network system (e.g., Plume Wi-Fi, Google Wi-Fi, Qualcomm Wi-Fi SON, etc.). In some cases, another type of standard or conventional Wi-Fi transmitter device may be used. In some instances, one or more of wireless communication devices 102A, 102B, 102C may be implemented as WAPs in a mesh network, while other wireless communication device(s) 102A, 102B, 102C are implemented as leaf devices (e.g., mobile devices, smart devices, etc.) that access the mesh network through one of the WAPs. In some cases, one or more of wireless communication devices 102A, 102B, 102C is a mobile device (e.g., a smartphone, a smart watch, a tablet, a laptop computer, etc.), a wireless-enabled device (e.g., a smart thermostat, a Wi-Fi enabled camera, a smart TV), or another type of device that communicates in a wireless network.

Wireless communication devices 102A, 102B, 102C may be implemented without Wi-Fi components; for example, other types of standard or non-standard wireless communication may be used for motion detection. In some cases, wireless communication devices 102A, 102B, 102C can be, or they may be part of, a dedicated motion detection system. For example, the dedicated motion detection system can include a hub device and one or more beacon devices (as remote sensor devices), and wireless communication devices 102A, 102B, 102C can be either a hub device or a beacon device in the motion detection system.

As shown in FIG. 1, wireless communication device 102C includes modem 112, processor 114, memory 116, and power unit 118; any of wireless communication devices 102A, 102B, 102C in wireless communication system 100 may include the same, additional, or different components, and the components may be configured to operate as shown in FIG. 1 or in another manner. In some implementations, modem 112, processor 114, memory 116, and power unit 118 of a wireless communication device are housed together in a common housing or other assembly. In some implementations, one or more of the components of a wireless communication device can be housed separately, for example, in a separate housing or other assembly.

Modem 112 can communicate (receive, transmit, or both) wireless signals. For example, modem 112 may be configured to communicate radio frequency (RF) signals formatted according to a wireless communication standard (e.g., Wi-Fi or Bluetooth). Modem 112 may be implemented as the example wireless network modem 112 shown in FIG. 1, or may be implemented in another manner, for example, with other types of components or subsystems. In some implementations, modem 112 includes a radio subsystem and a baseband subsystem. In some cases, the baseband subsystem and radio subsystem can be implemented on a common chip or chipset, or they may be implemented in a card or another type of assembled device. The baseband subsystem can be coupled to the radio subsystem, for example, by leads, pins, wires, or other types of connections.

In some cases, a radio subsystem in modem 112 can include one or more antennas and radio frequency circuitry. The radio frequency circuitry can include, for example, circuitry that filters, amplifies, or otherwise conditions analog signals, circuitry that up-converts baseband signals to RF signals, circuitry that down-converts RF signals to baseband signals, etc. Such circuitry may include, for example, filters, amplifiers, mixers, a local oscillator, etc. The radio subsystem can be configured to communicate radio frequency wireless signals on the wireless communication channels. As an example, the radio subsystem may include a radio chip, an RF front end, and one or more antennas. A radio subsystem may include additional or different components. In some implementations, the radio subsystem can be or include the radio electronics (e.g., RF front end, radio chip, or analogous components) from a conventional modem, for example, from a Wi-Fi modem, pico base station modem, etc. In some implementations, the antenna includes multiple antennas.

In some cases, a baseband subsystem in modem 112 can include, for example, digital electronics configured to process digital baseband data. As an example, the baseband subsystem may include a baseband chip. A baseband subsystem may include additional or different components. In some cases, the baseband subsystem may include a digital signal processor (DSP) device or another type of processor device. In some cases, the baseband system includes digital processing logic to operate the radio subsystem, to communicate wireless network traffic through the radio subsystem, to detect motion based on motion detection signals received through the radio subsystem or to perform other types of processes. For instance, the baseband subsystem may include one or more chips, chipsets, or other types of devices that are configured to encode signals and deliver the encoded signals to the radio subsystem for transmission, or to identify and analyze data encoded in signals from the radio subsystem (e.g., by decoding the signals according to a wireless communication standard, by processing the signals according to a motion detection process, or otherwise).

In some instances, the radio subsystem in modem 112 receives baseband signals from the baseband subsystem, up-converts the baseband signals to radio frequency (RF) signals, and wirelessly transmits the radio frequency signals (e.g., through an antenna). In some instances, the radio subsystem in modem 112 wirelessly receives radio frequency signals (e.g., through an antenna), down-converts the radio frequency signals to baseband signals, and sends the baseband signals to the baseband subsystem. The signals exchanged between the radio subsystem, and the baseband subsystem may be digital or analog signals. In some examples, the baseband subsystem includes conversion circuitry (e.g., a digital-to-analog converter, an analog-to-digital converter) and exchanges analog signals with the radio subsystem. In some examples, the radio subsystem includes conversion circuitry (e.g., a digital-to-analog converter, an analog-to-digital converter) and exchanges digital signals with the baseband subsystem.

In some cases, the baseband subsystem of modem 112 can communicate wireless network traffic (e.g., data packets) in the wireless communication network through the radio subsystem on one or more network traffic channels. The baseband subsystem of modem 112 may also transmit or receive (or both) signals (e.g., motion probe signals or motion detection signals) through the radio subsystem on a dedicated wireless communication channel. In some instances, the baseband subsystem generates motion probe signals for transmission, for example, to probe a space for motion. In some instances, the baseband subsystem processes received motion detection signals (signals based on motion probe signals transmitted through the space), for example, to detect motion of an object in a space.

Processor 114 can execute instructions, for example, to generate output data based on data inputs. The instructions can include programs, codes, scripts, or other types of data stored in memory. Additionally, or alternatively, the instructions can be encoded as pre-programmed or re-programmable logic circuits, logic gates, or other types of hardware or firmware components. Processor 114 may be or include a general-purpose microprocessor, as a specialized co-processor or another type of data processing apparatus. In some cases, processor 114 performs high level operation of the wireless communication device 102C. For example, processor 114 may be configured to execute or interpret software, scripts, programs, functions, executables, or other instructions stored in memory 116. In some implementations, processor 114 may be included in modem 112.

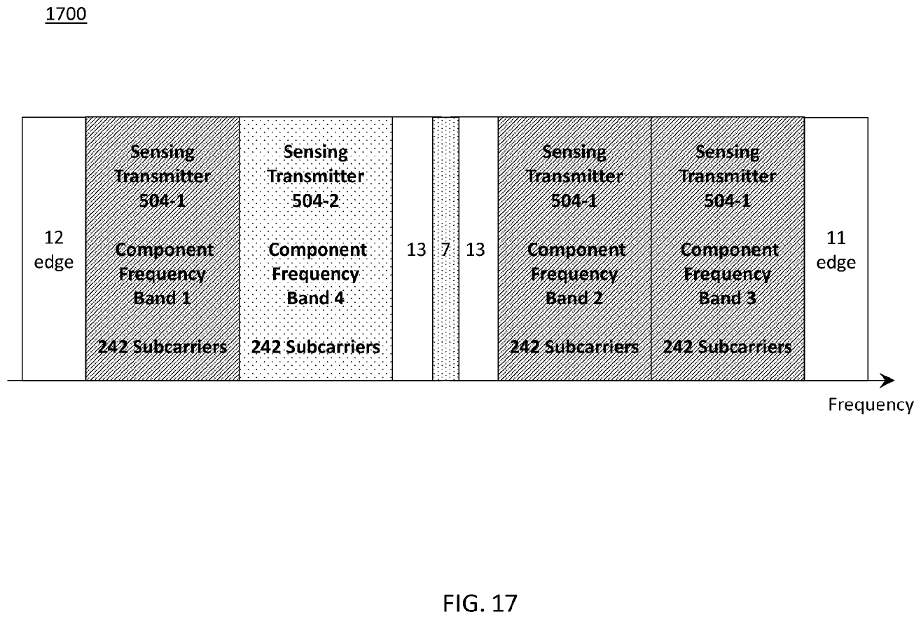

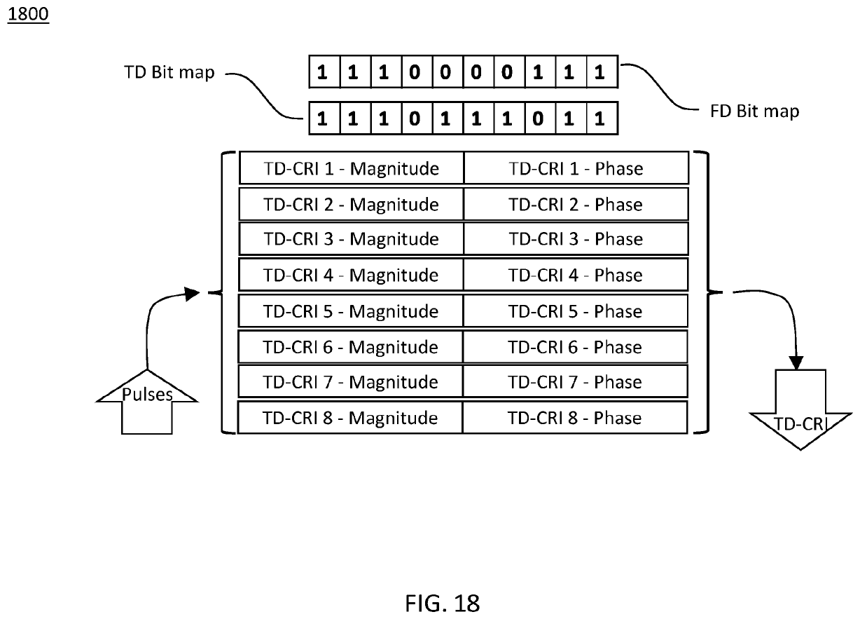

Memory 116 can include computer-readable storage media, for example, a volatile memory device, a non-volatile memory device, or both. Memory 116 can include one or more read-only memory devices, random-access memory devices, buffer memory devices, or a combination of these and other types of memory devices. In some instances, one or more components of the memory can be integrated or otherwise associated with another component of wireless communication device 102C. Memory 116 may store instructions that are executable by processor 114. For example, the instructions may include instructions for time-aligning signals using an interference buffer and a motion detection buffer, such as through one or more of the operations of the example processes as described in any of FIG. 17A, FIG. 17B, and FIG. 18.

Power unit 118 provides power to the other components of wireless communication device 102C. For example, the other components may operate based on electrical power provided by power unit 118 through a voltage bus or other connection. In some implementations, power unit 118 includes a battery or a battery system, for example, a rechargeable battery. In some implementations, power unit 118 includes an adapter (e.g., an AC adapter) that receives an external power signal (from an external source) and coverts the external power signal to an internal power signal conditioned for a component of wireless communication device 102C. Power unit 118 may include other components or operate in another manner.

In the example shown in FIG. 1, wireless communication devices 102A, 102B transmit wireless signals (e.g., according to a wireless network standard, a motion detection protocol, or otherwise). For instance, wireless communication devices 102A, 102B may broadcast wireless motion probe signals (e.g., reference signals, beacon signals, status signals, etc.), or they may send wireless signals addressed to other devices (e.g., a user equipment, a client device, a server, etc.), and the other devices (not shown) as well as wireless communication device 102C may receive the wireless signals transmitted by wireless communication devices 102A, 102B. In some cases, the wireless signals transmitted by wireless communication devices 102A, 102B are repeated periodically, for example, according to a wireless communication standard or otherwise.

In the example shown, wireless communication device 102C processes the wireless signals from wireless communication devices 102A, 102B to detect motion of an object in a space accessed by the wireless signals, to determine a location of the detected motion, or both. For example, wireless communication device 102C may perform one or more operations of the example processes described below with respect to any of FIG. 17A, FIG. 17B, and FIG. 18, or another type of process for detecting motion or determining a location of detected motion. The space accessed by the wireless signals can be an indoor or outdoor space, which may include, for example, one or more fully or partially enclosed areas, an open area without enclosure, etc. The space can be or can include an interior of a room, multiple rooms, a building, or the like. In some cases, the wireless communication system 100 can be modified, for instance, such that wireless communication device 102C can transmit wireless signals and wireless communication devices 102A, 102B can processes the wireless signals from wireless communication device 102C to detect motion or determine a location of detected motion.

The wireless signals used for motion detection can include, for example, a beacon signal (e.g., Bluetooth Beacons, Wi-Fi Beacons, other wireless beacon signals), another standard signal generated for other purposes according to a wireless network standard, or non-standard signals (e.g., random signals, reference signals, etc.) generated for motion detection or other purposes. In examples, motion detection may be carried out by analyzing one or more training fields carried by the wireless signals or by analyzing other data carried by the signal. In some examples data will be added for the express purpose of motion detection or the data used will nominally be for another purpose and reused or repurposed for motion detection. In some examples, the wireless signals propagate through an object (e.g., a wall) before or after interacting with a moving object, which may allow the moving object's movement to be detected without an optical line-of-sight between the moving object and the transmission or receiving hardware. Based on the received signals, wireless communication device 102C may generate motion detection data. In some instances, wireless communication device 102C may communicate the motion detection data to another device or system, such as a security system, which may include a control center for monitoring movement within a space, such as a room, building, outdoor area, etc.

In some implementations, wireless communication devices 102A, 102B can be modified to transmit motion probe signals (which may include, e.g., a reference signal, beacon signal, or another signal used to probe a space for motion) on a separate wireless communication channel (e.g., a frequency channel or coded channel) from wireless network traffic signals. For example, the modulation applied to the payload of a motion probe signal and the type of data or data structure in the payload may be known by wireless communication device 102C, which may reduce the amount of processing that wireless communication device 102C performs for motion sensing. The header may include additional information such as, for example, an indication of whether motion was detected by another device in wireless communication system 100, an indication of the modulation type, an identification of the device transmitting the signal, etc.

In the example shown in FIG. 1, wireless communication system 100 is a wireless mesh network, with wireless communication links between each of wireless communication devices 102. In the example shown, the wireless communication link between wireless communication device 102C and wireless communication device 102A can be used to probe motion detection field 110A, the wireless communication link between wireless communication device 102C and wireless communication device 102B can be used to probe motion detection field 110B, and the wireless communication link between wireless communication device 102A and wireless communication device 102B can be used to probe motion detection field 110C. In some instances, each wireless communication device 102 detects motion in motion detection fields 110 accessed by that device by processing received signals that are based on wireless signals transmitted by wireless communication devices 102 through motion detection fields 110. For example, when person 106 shown in FIG. 1 moves in motion detection field 110A and motion detection field 110C, wireless communication devices 102 may detect the motion based on signals they received that are based on wireless signals transmitted through respective motion detection fields 110. For instance, wireless communication device 102A can detect motion of person 106 in motion detection fields 110A, 110C, wireless communication device 102B can detect motion of person 106 in motion detection field 110C, and wireless communication device 102C can detect motion of person 106 in motion detection field 110A.

In some instances, motion detection fields 110 can include, for example, air, solid materials, liquids, or another medium through which wireless electromagnetic signals may propagate. In the example shown in FIG. 1, motion detection field 110A provides a wireless communication channel between wireless communication device 102A and wireless communication device 102C, motion detection field 110B provides a wireless communication channel between wireless communication device 102B and wireless communication device 102C, and motion detection field 110C provides a wireless communication channel between wireless communication device 102A and wireless communication device 102B. In some aspects of operation, wireless signals transmitted on a wireless communication channel (separate from or shared with the wireless communication channel for network traffic) are used to detect movement of an object in a space. The objects can be any type of static or moveable object and can be living or inanimate. For example, the object can be a human (e.g., person 106 shown in FIG. 1), an animal, an inorganic object, or another device, apparatus, or assembly), an object that defines all or part of the boundary of a space (e.g., a wall, door, window, etc.), or another type of object. In some implementations, motion information from the wireless communication devices may be analyzed to determine a location of the detected motion. For example, as described further below, one of wireless communication devices 102 (or another device communicably coupled to wireless communications devices 102) may determine that the detected motion is nearby a particular wireless communication device.

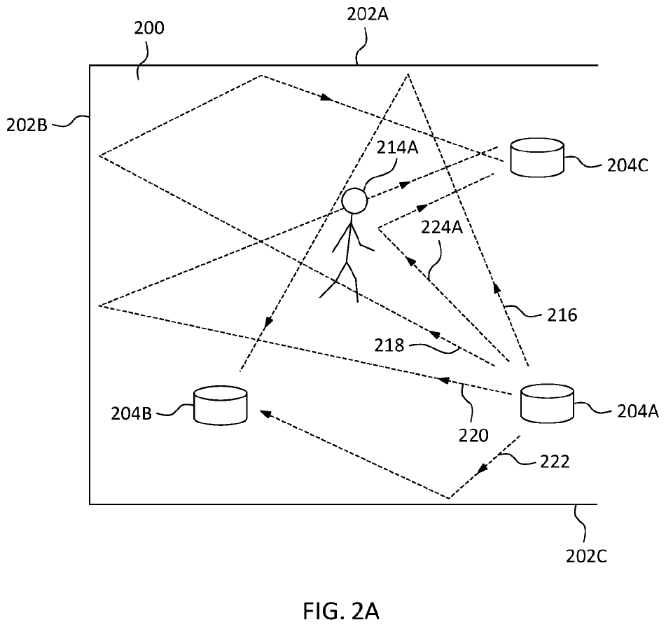

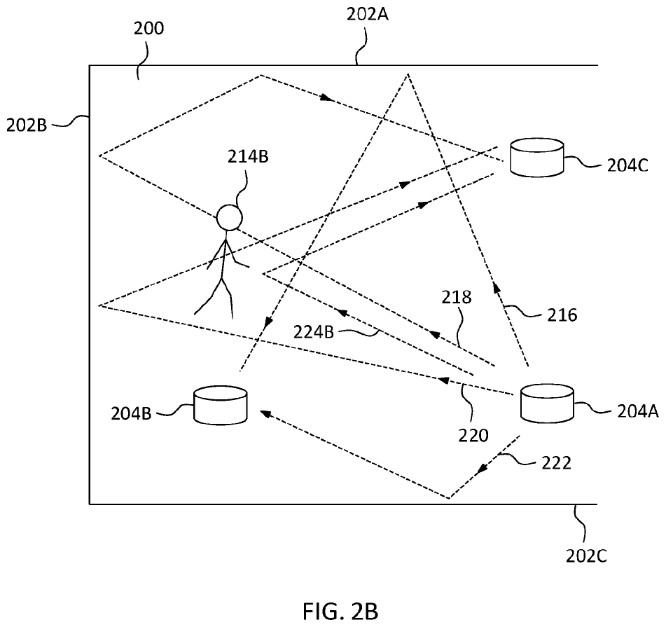

FIG. 2A and FIG. 2B are diagrams showing example wireless signals communicated between wireless communication devices 204A, 204B, 204C. Wireless communication devices 204A, 204B, 204C can be, for example, wireless communication devices 102A, 102B, 102C shown in FIG. 1, or other types of wireless communication devices. Wireless communication devices 204A, 204B, 204C transmit wireless signals through space 200. Space 200 can be completely or partially enclosed or open at one or more boundaries. Space 200 can be or can include an interior of a room, multiple rooms, a building, an indoor area, outdoor area, or the like. First wall 202A, second wall 202B, and third wall 202C at least partially enclose space 200 in the example shown.

In the example shown in FIG. 2A and FIG. 2B, wireless communication device 204A is operable to transmit wireless signals repeatedly (e.g., periodically, intermittently, at scheduled, unscheduled, or random intervals, etc.). Wireless communication devices 204B, 204C are operable to receive signals based on those transmitted by wireless communication device 204A. Wireless communication devices 204B, 204C each have a modem (e.g., modem 112 shown in FIG. 1) that is configured to process received signals to detect motion of an object in space 200.

As shown, an object is in first position 214A in FIG. 2A, and the object has moved to second position 214B in FIG. 2B. In FIG. 2A and FIG. 2B, the moving object in space 200 is represented as a human, but the moving object can be another type of object. For example, the moving object can be an animal, an inorganic object (e.g., a system, device, apparatus, or assembly), an object that defines all or part of the boundary of space 200 (e.g., a wall, door, window, etc.), or another type of object.

As shown in FIG. 2A and FIG. 2B, multiple example paths of the wireless signals transmitted from wireless communication device 204A are illustrated by dashed lines. Along first signal path 216, the wireless signal is transmitted from wireless communication device 204A and reflected off first wall 202A toward the wireless communication device 204B. Along second signal path 218, the wireless signal is transmitted from the wireless communication device 204A and reflected off second wall 202B and first wall 202A toward wireless communication device 204C. Along third signal path 220, the wireless signal is transmitted from the wireless communication device 204A and reflected off second wall 202B toward wireless communication device 204C. Along fourth signal path 222, the wireless signal is transmitted from the wireless communication device 204A and reflected off third wall 202C toward the wireless communication device 204B.

In FIG. 2A, along fifth signal path 224A, the wireless signal is transmitted from wireless communication device 204A and reflected off the object at first position 214A toward wireless communication device 204C. Between FIG. 2A and FIG. 2B, a surface of the object moves from first position 214A to second position 214B in space 200 (e.g., some distance away from first position 214A). In FIG. 2B, along sixth signal path 224B, the wireless signal is transmitted from wireless communication device 204A and reflected off the object at second position 214B toward wireless communication device 204C. Sixth signal path 224B depicted in FIG. 2B is longer than fifth signal path 224A depicted in FIG. 2A due to the movement of the object from first position 214A to second position 214B. In some examples, a signal path can be added, removed, or otherwise modified due to movement of an object in a space.

The example wireless signals shown in FIG. 2A and FIG. 2B may experience attenuation, frequency shifts, phase shifts, or other effects through their respective paths and may have portions that propagate in another direction, for example, through the first, second and third walls 202A, 202B, and 202C. In some examples, the wireless signals are radio frequency (RF) signals. The wireless signals may include other types of signals.

In the example shown in FIG. 2A and FIG. 2B, wireless communication device 204A can repeatedly transmit a wireless signal. In particular, FIG. 2A shows the wireless signal being transmitted from wireless communication device 204A at a first time, and FIG. 2B shows the same wireless signal being transmitted from wireless communication device 204A at a second, later time. The transmitted signal can be transmitted continuously, periodically, at random or intermittent times or the like, or a combination thereof. The transmitted signal can have a number of frequency components in a frequency bandwidth. The transmitted signal can be transmitted from wireless communication device 204A in an omnidirectional manner, in a directional manner or otherwise. In the example shown, the wireless signals traverse multiple respective paths in space 200, and the signal along each path may become attenuated due to path losses, scattering, reflection, or the like and may have a phase or frequency offset.

As shown in FIG. 2A and FIG. 2B, the signals from first to sixth paths 216, 218, 220, 222, 224A, and 224B combine at wireless communication device 204C and wireless communication device 204B to form received signals. Because of the effects of the multiple paths in space 200 on the transmitted signal, space 200 may be represented as a transfer function (e.g., a filter) in which the transmitted signal is input and the received signal is output. When an object moves in space 200, the attenuation or phase offset affected upon a signal in a signal path can change, and hence, the transfer function of space 200 can change. Assuming the same wireless signal is transmitted from wireless communication device 204A, if the transfer function of space 200 changes, the output of that transfer function—the received signal-will also change. A change in the received signal can be used to detect movement of an object.

Mathematically, a transmitted signal f(t) transmitted from the first wireless communication device 204A may be described according to Equation (1):

$$f ( t ) = {\sum }_{n = - ∞}^{∞} {c}_{n} {e}^{j {ω}_{n} t} \qquad ( 1 )$$

Where ωₙ represents the frequency of nth frequency component of the transmitted signal, cₙ represents the complex coefficient of the nth frequency component, and t represents time. With the transmitted signal f(t) being transmitted from the first wireless communication device 204A, an output signal rₖ(t) from a path k may be described according to Equation (2):

$${r}_{k} ( t ) = {\sum }_{n = - ∞}^{∞} {α}_{n , k} {c}_{n} {e}^{j ( {ω}_{n} t + {ϕ}_{n , k} )} \qquad ( 2 )$$

Where α_{n,k} represents an attenuation factor (or channel response; e.g., due to scattering, reflection, and path losses) for the nth frequency component along path k, and ϕ_{n,k} represents the phase of the signal for nth frequency component along path k. Then, the received signal R at a wireless communication device can be described as the summation of all output signals rₖ(t) from all paths to the wireless communication device, which is shown in Equation (3):

$$R = {\sum }_{k} {r}_{k} ( t ) \qquad ( 3 )$$

Substituting Equation (2) into Equation (3) renders the following Equation (4):

$$R = {\sum }_{k} {\sum }_{n = - ∞}^{∞} ( {α}_{n , k} {e}^{j {ϕ}_{n , k}} ) {c}_{n} {e}^{j {ω}_{n} t} \qquad ( 4 )$$

The received signal R at a wireless communication device can then be analyzed. The received signal R at a wireless communication device can be transformed to the frequency domain, for example, using a Fast Fourier Transform (FFT) or another type of algorithm. The transformed signal can represent the received signal R as a series of n complex values, one for each of the respective frequency components (at the n frequencies (n). For a frequency component at frequency ωₙ, a complex value Hₙ may be represented as follows in Equation (5):

$${H}_{n} = {\sum }_{k} {c}_{n} {α}_{n , k} {e}^{j {ϕ}_{n , k}} \qquad ( 5 )$$

The complex value Hₙ for a given frequency component on indicates a relative magnitude and phase offset of the received signal at that frequency component ωₙ. When an object moves in the space, the complex value Hₙ changes due to the channel response α_{n,k} of the space changing. Accordingly, a change detected in the channel response can be indicative of movement of an object within the communication channel. In some instances, noise, interference, or other phenomena can influence the channel response detected by the receiver, and the motion detection system can reduce or isolate such influences to improve the accuracy and quality of motion detection capabilities. In some implementations, the overall channel response can be represented as follows in Equation (6):

$${h}_{ch} = {\sum }_{k} {\sum }_{n = - ∞}^{∞} {α}_{n , k} \qquad ( 6 )$$

In some instances, the channel response h_{ch} for a space can be determined, for example, based on the mathematical theory of estimation. For instance, a reference signal Ref can be modified with candidate channel responses (h_{ch}), and then a maximum likelihood approach can be used to select the candidate channel which gives best match to the received signal (R_{evd}). In some cases, an estimated received signal (Rcvd) is obtained from the convolution of the reference signal (R_{ef}) with the candidate channel responses (h_{ch}), and then the channel coefficients of the channel response (h_{ch}) are varied to minimize the squared error of the estimated received signal (\hat{R}_{cvd}). This can be mathematically illustrated as follows in Equation (7):

$${R}_{cvd} = {R}_{ef} ⊗ {h}_{ch} = {\sum }_{k = - 𝔪}^{𝔪} {R}_{ef} ( n - k ) {h}_{ch} ( k ) \qquad ( 7 )$$

with the optimization criterion

$${min}_{{h}_{ch}} \sum {( {\hat{R}}_{cvd} - {R}_{cvd} )}^{2}$$

The minimizing, or optimizing, process can utilize an adaptive filtering technique, such as Least Mean Squares (LMS), Recursive Least Squares (RLS), Batch Least Squares (BLS), etc. The channel response can be a Finite Impulse Response (FIR) filter, Infinite Impulse Response (IIR) filter, or the like. As shown in the equation above, the received signal can be considered as a convolution of the reference signal and the channel response. The convolution operation means that the channel coefficients possess a degree of correlation with each of the delayed replicas of the reference signal. The convolution operation as shown in the equation above, therefore shows that the received signal appears at different delay points, each delayed replica being weighted by the channel coefficient.

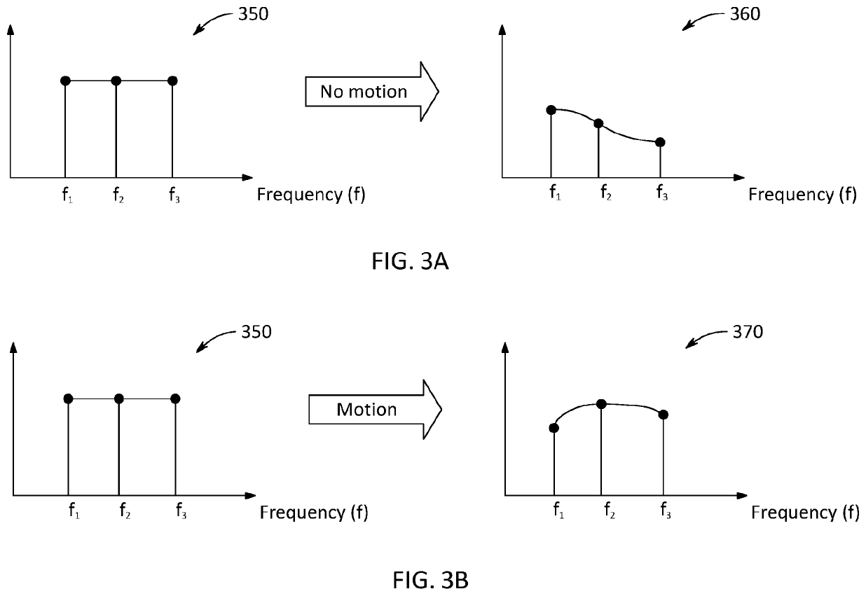

FIG. 3A and FIG. 3B are plots showing examples of channel responses 360, 370 computed from the wireless signals communicated between wireless communication devices 204A, 204B, 204C in FIG. 2A and FIG. 2B. FIG. 3A and FIG. 3B also show frequency domain representation 350 of an initial wireless signal transmitted by wireless communication device 204A. In the examples shown, channel response 360 in FIG. 3A represents the signals received by wireless communication device 204B when there is no motion in space 200, and channel response 370 in FIG. 3B represents the signals received by wireless communication device 204B in FIG. 2B after the object has moved in space 200.

In the example shown in FIG. 3A and FIG. 3B, for illustration purposes, wireless communication device 204A transmits a signal that has a flat frequency profile (the magnitude of each frequency component f₁, f₂, and f₃ is the same), as shown in frequency domain representation 350. Because of the interaction of the signal with space 200 (and the objects therein), the signals received at wireless communication device 204B that are based on the signal sent from wireless communication device 204A look different from the transmitted signal. In this example, where the transmitted signal has a flat frequency profile, the received signal represents the channel response of space 200. As shown in FIG. 3A and FIG. 3B, channel responses 360, 370 are different from frequency domain representation 350 of the transmitted signal. When motion occurs in space 200, a variation in the channel response will also occur. For example, as shown in FIG. 3B, channel response 370 that is associated with motion of object in space 200 varies from channel response 360 that is associated with no motion in space 200.

Furthermore, as an object moves within space 200, the channel response may vary from channel response 370. In some cases, space 200 can be divided into distinct regions and the channel responses associated with each region may share one or more characteristics (e.g., shape), as described below. Thus, motion of an object within different distinct regions can be distinguished, and the location of detected motion can be determined based on an analysis of channel responses.

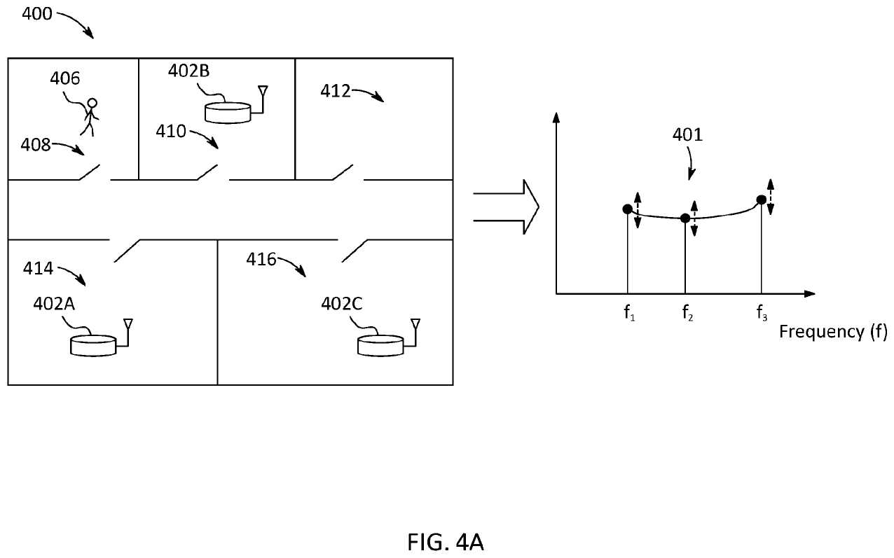

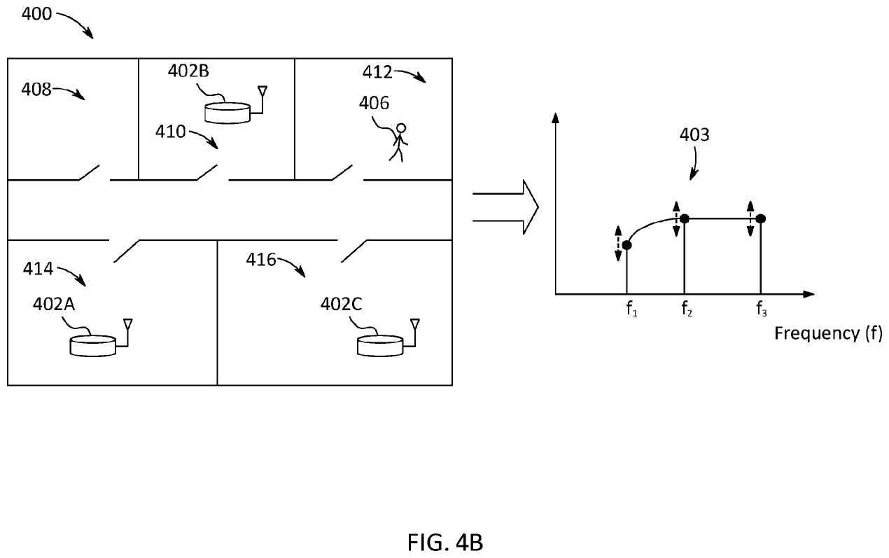

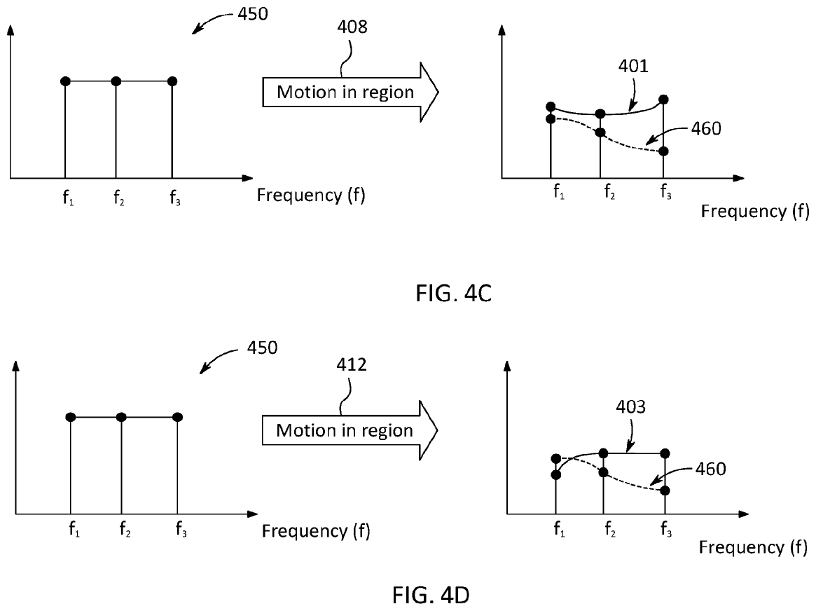

FIG. 4A and FIG. 4B are diagrams showing example channel responses 401, 403 associated with motion of object 406 in distinct regions 408, 412 of space 400. In the examples shown, space 400 is a building, and space 400 is divided into a plurality of distinct regions-first region 408, second region 410, third region 412, fourth region 414, and fifth region 416. Space 400 may include additional or fewer regions, in some instances. As shown in FIGS. 4A and 4B, the regions within space 400 may be defined by walls between rooms. In addition, the regions may be defined by ceilings between floors of a building. For example, space 400 may include additional floors with additional rooms. In addition, in some instances, the plurality of regions of a space can be or include a number of floors in a multistory building, a number of rooms in the building, or a number of rooms on a particular floor of the building. In the example shown in FIG. 4A, an object located in first region 408 is represented as person 106, but the moving object can be another type of object, such as an animal or an inorganic object.

In the example shown, wireless communication device 402A is located in fourth region 414 of space 400, wireless communication device 402B is located in second region 410 of space 400, and wireless communication device 402C is located in fifth region 416 of space 400. Wireless communication devices 402 can operate in the same or similar manner as wireless communication devices 102 of FIG. 1. For instance, wireless communication devices 402 may be configured to transmit and receive wireless signals and detect whether motion has occurred in space 400 based on the received signals. As an example, wireless communication devices 402 may periodically or repeatedly transmit motion probe signals through space 400, and receive signals based on the motion probe signals. Wireless communication devices 402 can analyze the received signals to detect whether an object has moved in space 400, such as, for example, by analyzing channel responses associated with space 400 based on the received signals. In addition, in some implementations, wireless communication devices 402 can analyze the received signals to identify a location of detected motion within space 400. For example, wireless communication devices 402 can analyze characteristics of the channel response to determine whether the channel responses share the same or similar characteristics to channel responses known to be associated with first to fifth regions 408, 410, 412, 414, 416 of space 400.

In the examples shown, one (or more) of wireless communication devices 402 repeatedly transmits a motion probe signal (e.g., a reference signal) through space 400. The motion probe signals may have a flat frequency profile in some instances, wherein the magnitude of each frequency component f₁, f₂, and f₃. For example, the motion probe signals may have a frequency response similar to frequency domain representation 350 shown in FIG. 3A and FIG. 3B. The motion probe signals may have a different frequency profile in some instances. Because of the interaction of the reference signal with space 400 (and the objects therein), the signals received at another wireless communication device 402 that are based on the motion probe signal transmitted from the other wireless communication device 402 are different from the transmitted reference signal.

Based on the received signals, wireless communication devices 402 can determine a channel response for space 400. When motion occurs in distinct regions within the space, distinct characteristics may be seen in the channel responses. For example, while the channel responses may differ slightly for motion within the same region of space 400, the channel responses associated with motion in distinct regions may generally share the same shape or other characteristics. For instance, channel response 401 of FIG. 4A represents an example channel response associated with motion of object 406 in first region 408 of space 400, while channel response 403 of FIG. 4B represents an example channel response associated with motion of object 406 in third region 412 of space 400. Channel responses 401, 403 are associated with signals received by the same wireless communication device 402 in space 400.

FIG. 4C and FIG. 4D are plots showing channel responses 401, 403 of FIG. 4A and FIG. 4B overlaid on channel response 460 associated with no motion occurring in space 400. FIG. 4C-FIG. 4D also show frequency domain representation 450 of an initial wireless signal transmitted by one or more of wireless communication devices 402A, 402B, 402C. When motion occurs in space 400, a variation in the channel response will occur relative to channel response 460 associated with no motion, and thus, motion of an object in space 400 can be detected by analyzing variations in the channel responses. In addition, a relative location of the detected motion within space 400 can be identified. For example, the shape of channel responses associated with motion can be compared with reference information (e.g., using a trained AI model) to categorize the motion as having occurred within a distinct region of space 400.

When there is no motion in space 400 (e.g., when object 406 is not present), wireless communication device 402 may compute channel response 460 associated with no motion. Slight variations may occur in the channel response due to a number of factors; however, multiple channel responses 460 associated with different periods of time may share one or more characteristics. In the example shown, channel response 460 associated with no motion has a decreasing frequency profile (the magnitude of each frequency component f₁, f₂, and f₃ is less than the previous). The profile of channel response 460 may differ in some instances (e.g., based on different room layouts or placement of wireless communication devices 402).

When motion occurs in space 400, a variation in the channel response will occur. For instance, in the examples shown in FIG. 4C and FIG. 4D, channel response 401 associated with motion of object 406 in first region 408 differs from channel response 460 associated with no motion and channel response 403 associated with motion of object 406 in third region 412 differs from channel response 460 associated with no motion. Channel response 401 has a concave-parabolic frequency profile (the magnitude of the middle frequency component f₂ is less than the outer frequency components f₁ and f₃), while channel response 403 has a convex-asymptotic frequency profile (the magnitude of the middle frequency component f₂ is greater than the outer frequency components f₁ and f₃). The profiles of channel responses 401, 403 may differ in some instances (e.g., based on different room layouts or placement of the wireless communication devices 402).

Analyzing channel responses may be considered similar to analyzing a digital filter. In other words, a channel response has been formed through the reflections of objects in a space as well as reflections created by a moving or static human. When a reflector (e.g., a human) moves, it changes the channel response. This may translate to a change in equivalent taps of a digital filter, which can be thought of as having poles and zeros (poles amplify the frequency components of a channel response and appear as peaks or high points in the response, while zeros attenuate the frequency components of a channel response and appear as troughs, low points, or nulls in the response). A changing digital filter can be characterized by the locations of its peaks and troughs, and a channel response may be characterized similarly by its peaks and troughs. For example, in some implementations, analyzing nulls and peaks in the frequency components of a channel response (e.g., by marking their location on the frequency axis and their magnitude), motion can be detected.

In some implementations, a time series aggregation can be used to detect motion. A time series aggregation may be performed by observing the features of a channel response over a moving window and aggregating the windowed result by using statistical measures (e.g., mean, variance, principal components, etc.). During instances of motion, the characteristic digital-filter features would be displaced in location and flip-flop between some values due to the continuous change in the scattering scene. That is, an equivalent digital filter exhibits a range of values for its peaks and nulls (due to the motion). By looking this range of values, unique profiles (in examples profiles may also be referred to as signatures) may be identified for distinct regions within a space.

In some implementations, an artificial intelligence (AI) model may be used to process data. AI models may be of a variety of types, for example linear regression models, logistic regression models, linear discriminant analysis models, decision tree models, naïve bayes models, K-nearest neighbors models, learning vector quantization models, support vector machines, bagging and random forest models, and deep neural networks. In general, all AI models aim to learn a function which provides the most precise correlation between input values and output values and are trained using historic sets of inputs and outputs that are known to be correlated. In examples, artificial intelligence may also be referred to as machine learning.

In some implementations, the profiles of the channel responses associated with motion in distinct regions of space 400 can be learned. For example, machine learning may be used to categorize channel response characteristics with motion of an object within distinct regions of a space. In some cases, a user associated with wireless communication devices 402 (e.g., an owner or other occupier of space 400) can assist with the learning process. For instance, referring to the examples shown in FIG. 4A and FIG. 4B, the user can move in each of first to fifth regions 408, 410, 412, 414, 416 during a learning phase and may indicate (e.g., through a user interface on a mobile computing device) that he/she is moving in one of the particular regions in space 400. For example, while the user is moving through first region 408 (e.g., as shown in FIG. 4A) the user may indicate on a mobile computing device that he/she is in first region 408 (and may name the region as “bedroom”, “living room”, “kitchen”, or another type of room of a building, as appropriate). Channel responses may be obtained as the user moves through the region, and the channel responses may be “tagged” with the user's indicated location (region). The user may repeat the same process for the other regions of space 400. The term “tagged” as used herein may refer to marking and identifying channel responses with the user's indicated location or any other information.

The tagged channel responses can then be processed (e.g., by machine learning software) to identify unique characteristics of the channel responses associated with motion in the distinct regions. Once identified, the identified unique characteristics may be used to determine a location of detected motion for newly computed channel responses. For example, an AI model may be trained using the tagged channel responses, and once trained, newly computed channel responses can be input to the AI model, and the AI model can output a location of the detected motion. For example, in some cases, mean, range, and absolute values are input to an AI model. In some instances, magnitude and phase of the complex channel response itself may be input as well. These values allow the AI model to design arbitrary front-end filters to pick up the features that are most relevant to making accurate predictions with respect to motion in distinct regions of a space. In some implementations, the AI model is trained by performing a stochastic gradient descent. For instance, channel response variations that are most active during a certain zone may be monitored during the training, and the specific channel variations may be weighted heavily (by training and adapting the weights in the first layer to correlate with those shapes, trends, etc.). The weighted channel variations may be used to create a metric that activates when a user is present in a certain region.

For extracted features like channel response nulls and peaks, a time-series (of the nulls/peaks) may be created using an aggregation within a moving window, taking a snapshot of few features in the past and present, and using that aggregated value as input to the network. Thus, the network, while adapting its weights, will be trying to aggregate values in a certain region to cluster them, which can be done by creating a logistic classifier based decision surfaces. The decision surfaces divide different clusters and subsequent layers can form categories based on a single cluster or a combination of clusters.

In some implementations, an AI model includes two or more layers of inference. The first layer acts as a logistic classifier which can divide different concentration of values into separate clusters, while the second layer combines some of these clusters together to create a category for a distinct region. Additional, subsequent layers can help in extending the distinct regions over more than two categories of clusters. For example, a fully-connected AI model may include an input layer corresponding to the number of features tracked, a middle layer corresponding to the number of effective clusters (through iterating between choices), and a final layer corresponding to different regions. Where complete channel response information is input to the AI model, the first layer may act as a shape filter that can correlate certain shapes. Thus, the first layer may lock to a certain shape, the second layer may generate a measure of variation happening in those shapes, and third and subsequent layers may create a combination of those variations and map them to different regions within the space. The output of different layers may then be combined through a fusing layer.

### B. Wi-Fi Sensing System Example Methods and Apparatus

Section B describes systems and methods that are useful for a Wi-Fi sensing system configurated to send sensing transmissions and make sensing measurements.

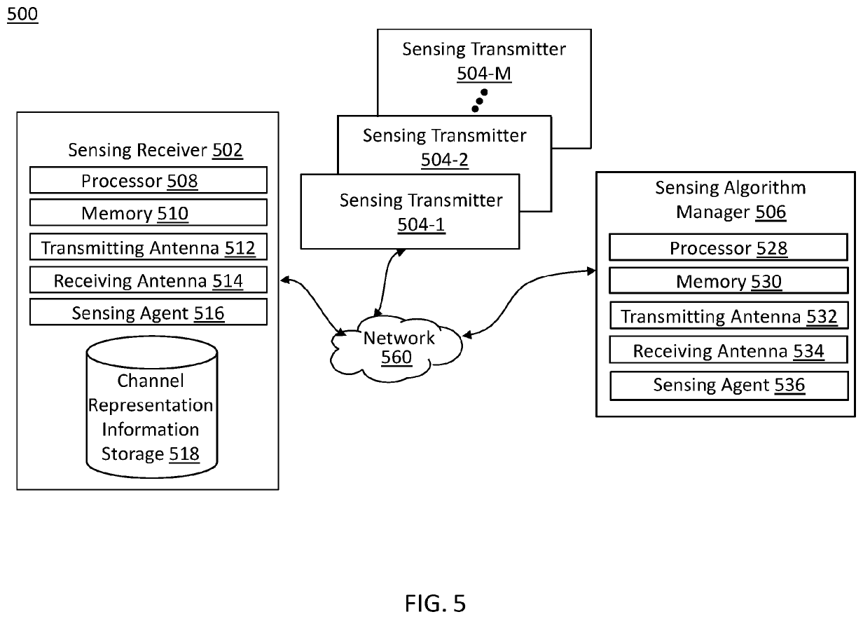

FIG. 5 depicts an implementation of some of an architecture of an implementation of system 500 for Wi-Fi sensing, according to some embodiments.

System 500 may include sensing receiver 502, plurality of sensing transmitter 504-(1-M), sensing algorithm manager 506, and network 560 enabling communication between the system components for information exchange. System 500 may be an example or instance of wireless communication system 100, and network 560 may be an example or instance of wireless network or cellular network, details of which are provided with reference to FIG. 1 and its accompanying description.

According to an embodiment, sensing receiver 502 may be configured to receive a sensing transmission (for example, from each of plurality of sensing transmitters 504-(1-M)) and perform one or more measurements (for example, channel state information (CSI)) useful for Wi-Fi sensing. These measurements may be known as sensing measurements. The sensing measurements may be processed to achieve a sensing result of system 500, such as detecting motions or gestures. In an embodiment, sensing receiver 502 may be an AP. In some embodiments, sensing receiver 502 may take a role of sensing initiator.

According to an implementation, sensing receiver 502 may be implemented by a device, such as wireless communication device 102 shown in FIG. 1. In some implementations, sensing receiver 502 may be implemented by a device, such as wireless communication device 204 shown in FIG. 2A and FIG. 2B. Further, sensing receiver 502 may be implemented by a device, such as wireless communication device 402 shown in FIG. 4A and FIG. 4B. In an implementation, sensing receiver 502 may coordinate and control communication among plurality of sensing transmitters 504-(1-M). According to an implementation, sensing receiver 502 may be enabled to control a measurement campaign to ensure that required sensing transmissions are made at a required time and to ensure an accurate determination of sensing measurement. In some embodiments, sensing receiver 502 may process sensing measurements to achieve the sensing result of system 500. In some embodiments, sensing receiver 502 may be configured to transmit sensing measurements to sensing algorithm manager 506, and sensing algorithm manager 506 may be configured to process the sensing measurements to achieve the sensing result of system 500.

Referring again to FIG. 5, in some embodiments, each of plurality of sensing transmitters 504-(1-M) may form a part of a basic service set (BSS) and may be configured to send a sensing transmission to sensing receiver 502 based on which, one or more sensing measurements (for example, CSI) may be performed for Wi-Fi sensing. In an embodiment, each of plurality of sensing transmitters 504-(1-M) may be a station. According to an implementation, each of plurality of sensing transmitters 504-(1-M) may be implemented by a device, such as wireless communication device 102 shown in FIG. 1. In some implementations, each of plurality of sensing transmitters 504-(1-M) may be implemented by a device, such as wireless communication device 204 shown in FIG. 2A and FIG. 2B. Further, each of plurality of sensing transmitters 504-(1-M) may be implemented by a device, such as wireless communication device 402 shown in FIG. 4A and FIG. 4B. In some implementations, communication between sensing receiver 502 and each of plurality of sensing transmitters 504-(1-M) may happen via station management entity (SME) and MAC layer management entity (MLME) protocols.

In some embodiments, sensing algorithm manager 506 may be configured to receive sensing measurements from sensing receiver 502 and process the sensing measurements. In an example, sensing algorithm manager 506 may process and analyze the sensing measurements to identify one or more features of interest. According to some implementations, sensing algorithm manager 506 may include/execute a sensing algorithm. In an embodiment, sensing algorithm manager 506 may be a station. In some embodiments, sensing algorithm manager 506 may be an AP. According to an implementation, sensing algorithm manager 506 may be implemented by a device, such as wireless communication device 102 shown in FIG. 1. In some implementations, sensing algorithm manager 506 may be implemented by a device, such as wireless communication device 204 shown in FIG. 2A and FIG. 2B. Further, sensing algorithm manager 506 may be implemented by a device, such as wireless communication device 402 shown in FIG. 4A and FIG. 4B. In some embodiments, sensing algorithm manager 506 may be any computing device, such as a desktop computer, a laptop, a tablet computer, a mobile device, a personal digital assistant (PDA) or any other computing device. In embodiments, sensing algorithm manager 506 may take a role of sensing initiator where a sensing algorithm determines a measurement campaign and the sensing measurements required to fulfill the measurement campaign. Sensing algorithm manager 506 may communicate the sensing measurements required to fulfill the measurement campaign to sensing receiver 502 to coordinate and control communication among plurality of sensing transmitters 504-(1-M).

Although sensing algorithm manager 506 is shown in FIG. 5 as a functional block separate from sensing receiver 502 and plurality of sensing transmitters 504-(1-M), in an embodiment of system 500, sensing algorithm manager 506 may be implemented by either sensing receiver 502 or one of plurality of sensing transmitters 504-(1-M).

Referring to FIG. 5, in more detail, sensing receiver 502 may include processor 508 and memory 510. For example, processor 508 and memory 510 of sensing receiver 502 may be processor 114 and memory 116, respectively, as shown in FIG. 1. In an embodiment, sensing receiver 502 may further include transmitting antenna(s) 512, receiving antenna(s) 514, and sensing agent 516. In some embodiments, an antenna may be used to both transmit and receive signals in a half-duplex format. When the antenna is transmitting, it may be referred to as transmitting antenna 512, and when the antenna is receiving, it may be referred to as receiving antenna 514. It is understood by a person of normal skill in the art that the same antenna may be transmitting antenna 512 in some instances and receiving antenna 514 in other instances. In the case of an antenna array, one or more antenna elements may be used to transmit or receive a signal, for example, in a beamforming environment. In some examples, a group of antenna elements used to transmit a composite signal may be referred to as transmitting antenna 512, and a group of antenna elements used to receive a composite signal may be referred to as receiving antenna 514. In some examples, each antenna is equipped with its own transmission and receive paths, which may be alternately switched to connect to the antenna depending on whether the antenna is operating as transmitting antenna 512 or receiving antenna 514.

In an implementation, sensing agent 516 may be responsible for receiving sensing transmissions and associated transmission parameters, calculating sensing measurements, and processing sensing measurements to fulfill a sensing result. In some implementations, receiving sensing transmissions and associated transmission parameters, and calculating sensing measurements may be carried out by an algorithm running in the Medium Access Control (MAC) layer of sensing receiver 502 and processing sensing measurements to fulfill a sensing result may be carried out by an algorithm running in the application layer of sensing receiver 502. In examples, the algorithm running in the application layer of sensing receiver 502 is known as Wi-Fi sensing agent, sensing application, or sensing algorithm. In some implementations, the algorithm running in the MAC layer of sensing receiver 502 and the algorithm running in the application layer of sensing receiver 502 may run separately on processor 508. In an implementation, sensing agent 516 may pass physical layer parameters (e.g., such as CSI) from the MAC layer of sensing receiver 502 to the application layer of sensing receiver 502 and may use the physical layer parameters to detect one or more features of interest. In an example, the application layer may operate on the physical layer parameters and form services or features, which may be presented to an end-user. According to an implementation, communication between the MAC layer of sensing receiver 502 and other layers or components may take place based on communication interfaces, such as MLME interface and a data interface. According to some implementations, sensing agent 516 may include/execute a sensing algorithm. In an implementation, sensing agent 516 may process and analyze sensing measurements using the sensing algorithm and identify one or more features of interest. Further, sensing agent 516 may be configured to determine a number and timing of sensing transmissions and sensing measurements for the purpose of Wi-Fi sensing. In some implementations, sensing agent 516 may be configured to transmit sensing measurements to sensing algorithm manager 506 for further processing.

In an implementation, sensing agent 516 may be configured to cause at least one transmitting antenna of transmitting antenna(s) 512 to transmit messages to plurality of sensing transmitters 504-(1-M). Further, sensing agent 516 may be configured to receive, via at least one receiving antenna of receiving antennas(s) 514, messages from plurality of sensing transmitters 504-(1-M). In an example, sensing agent 516 may be configured to make sensing measurements based on one or more sensing transmissions received from plurality of sensing transmitters 504-(1-M). According to an implementation, sensing agent 516 may be configured to process and analyze the sensing measurements to identify one or more features of interest.

According to some embodiments, sensing receiver 502 may include channel representation information storage 518. In an implementation, channel representation information storage 518 may store information related to sensing measurements that represent a state of the propagation channels between sensing receiver 502 and plurality of sensing transmitters 504-(1-M). In an example, channel representation information storage 518 may store one or more of CSI, full time-domain channel representation information (TD-CRI), filtered TD-CRI, and reduced filtered TD-CRI. Information related to the sensing measurements stored in channel representation information storage 518 may be periodically or dynamically updated as required. In an implementation, channel representation information storage 518 may include any type or form of storage, such as a database or a file system or coupled to memory 510.

Referring again to FIG. 5, sensing algorithm manager 506 may include processor 528 and memory 530. For example, processor 528 and memory 530 of sensing algorithm manager 506 may be processor 114 and memory 116, respectively, as shown in FIG. 1. In an embodiment, sensing algorithm manager 506 may further include transmitting antenna(s) 532, receiving antenna(s) 534, and sensing agent 536. In an implementation, sensing agent 536 may be a block that passes physical layer parameters from the MAC of sensing algorithm manager 506 to application layer programs. Sensing agent 536 may be configured to cause at least one transmitting antenna of transmitting antenna(s) 532 and at least one receiving antenna of receiving antennas(s) 534 to exchange messages with sensing receiver 502. According to some implementations, sensing agent 536 may be responsible for receiving sensing measurements from sensing receiver 502 and processing the sensing measurements to obtain a sensing result. Sensing agent 536 may include/execute a sensing algorithm. In an implementation, sensing agent 536 may process and analyze sensing measurements using the sensing algorithm, and obtain the sensing result.

In some embodiments, an antenna may be used to both transmit and receive in a half-duplex format. When the antenna is transmitting, it may be referred to as transmitting antenna 532, and when the antenna is receiving, it may be referred to as receiving antenna 534. It is understood by a person of normal skill in the art that the same antenna may be transmitting antenna 532 in some instances and receiving antenna 534 in other instances. In the case of an antenna array, one or more antenna elements may be used to transmit or receive a signal, for example, in a beamforming environment. In some examples, a group of antenna elements used to transmit a composite signal may be referred to as transmitting antenna 532, and a group of antenna elements used to receive a composite signal may be referred to as receiving antenna 534. In some examples, each antenna is equipped with its own transmission and receive paths, which may be alternately switched to connect to the antenna depending on whether the antenna is operating as transmitting antenna 532 or receiving antenna 534.

In an embodiment where sensing algorithm manager 506 is implemented by sensing receiver 502 then processor 528 may be implemented by processor 508, memory 530 may be implemented by memory 510, transmitting antenna 532 may implemented by transmitting antenna 512, receiving antenna 534 may implemented by receiving antenna 514, and sensing agent 536 may be implemented by sensing agent 516. In examples where sensing algorithm manager 506 receives signals from sensing receiver 502 or where sensing receiver 502 receives signals from sensing algorithm manager 506 then this may be implemented without transmission over the air.

According to one or more implementations, communications in network 560 may be governed by one or more of the 802.11 family of standards developed by IEEE. Some example IEEE standards may include IEEE 802.11-2020, IEEE 802.11ax-2021, IEEE 802.11me, IEEE 802.11az and IEEE 802.11be. IEEE 802.11-2020 and IEEE 802.11ax-2021 are fully-ratified standards whilst IEEE 802.11me reflects an ongoing maintenance update to the IEEE 802.11-2020 standard and IEEE 802.11be defines the next generation of standard. IEEE 802.11az is an extension of the IEEE 802.11-2020 and IEEE 802.11ax-2021 standards which adds new functionality. In some implementations, communications may be governed by other standards (other or additional IEEE standards or other types of standards). In some embodiments, parts of network 560 which are not required by system 500 to be governed by one or more of the 802.11 family of standards may be implemented by an instance of any type of network, including wireless network or cellular network.

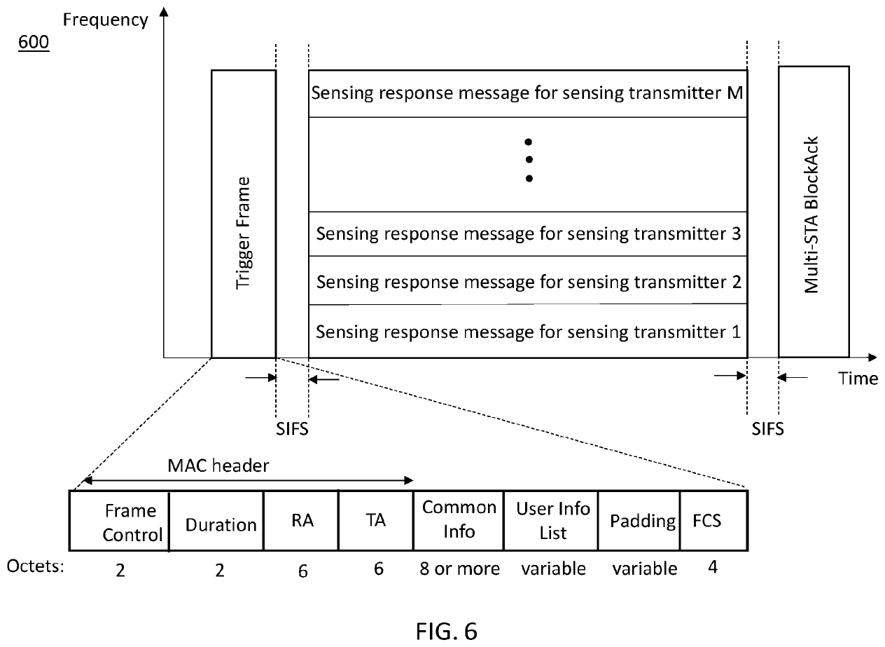

Further, IEEE 802.11ax adopted OFDMA, which allows sensing receiver 502 to simultaneously transmit data to all participating devices, such as plurality of sensing transmitters 504-(1-M), and vice versa using a single TXOP. The efficiency of OFDMA depends on how sensing receiver 502 schedules channel resources (interchangeably referred to as resource units (RUS)) among plurality of sensing transmitters 504-(1-M) and configures transmission parameters. Uplink OFDMA (UL-OFDMA) transmission procedure of IEEE 802.11ax and a trigger frame format are depicted in FIG. 6. According to IEEE 802.11ax, every uplink multiuser transmission follows a trigger frame, the format of which is depicted in FIG. 6. As can be seen in FIG. 6, a sensing transmission (i.e., sensing response message) follows the trigger frame (also referred to as UL-OFDMA sensing trigger message) after one SIFS. In an example, the duration of SIFS is 10 μs. The main purpose of the trigger frame is to solicit an immediate response of multiuser PPDUs from plurality of sensing transmitters 504-(1-M). According to an example, the trigger frame may specify common synchronization parameters to plurality of sensing transmitters 504-(1-M) for the TXOP along with a map to RUs for each sensing transmitter. The map allows the OFDMA to function without any interference. After the sensing transmissions are completed, a Multi-STA BlockAck may be sent to corresponding sensing transmitters 504-(1-M). A message controlled by the trigger frame generally follows a time-frequency message pattern, as shown in FIG. 6. The trigger frame includes a Common Info field, User Info List field, and various other fields.

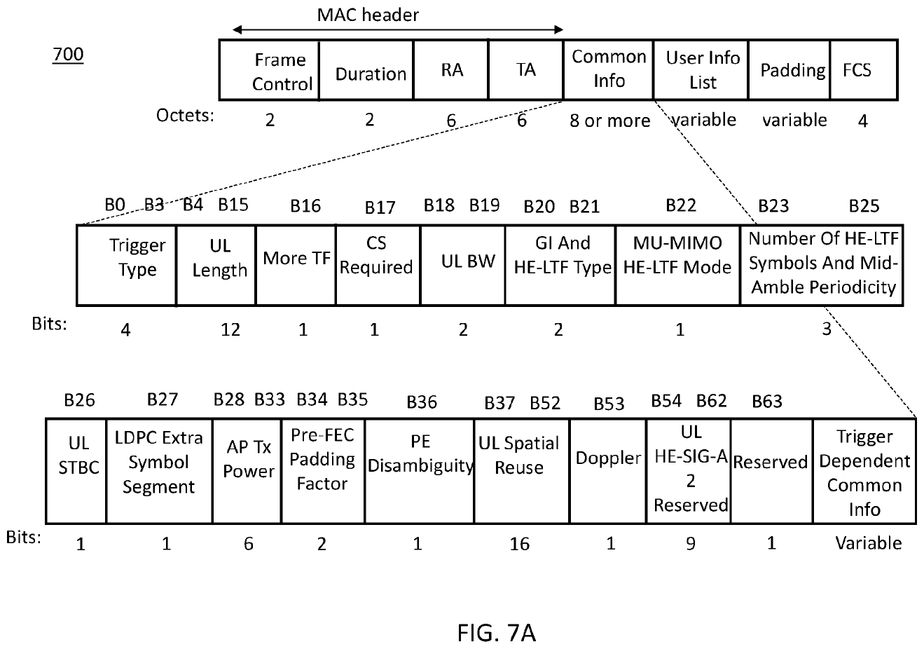

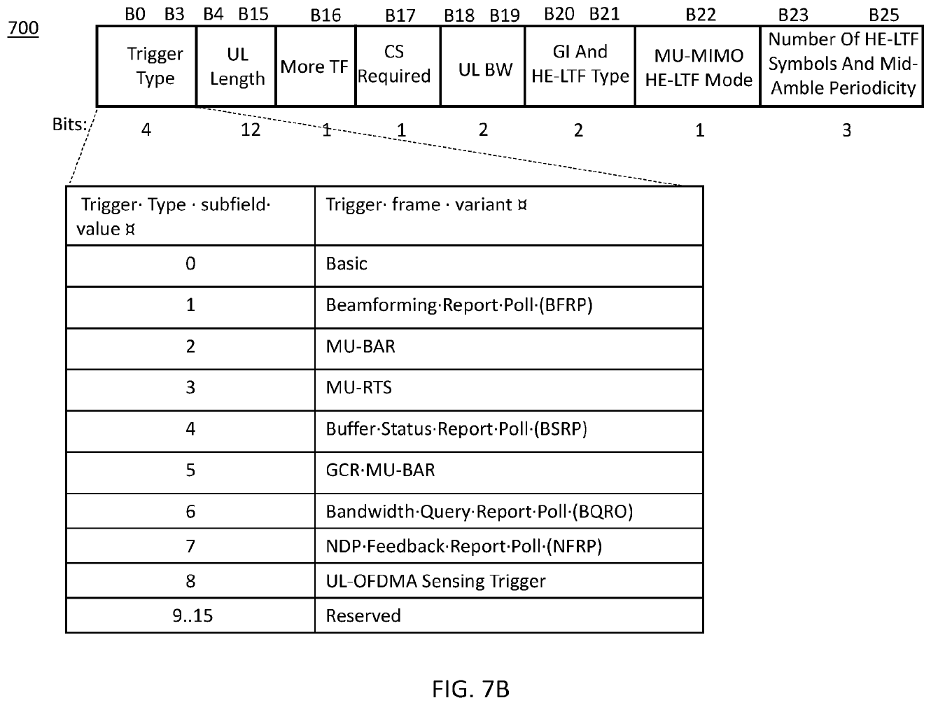

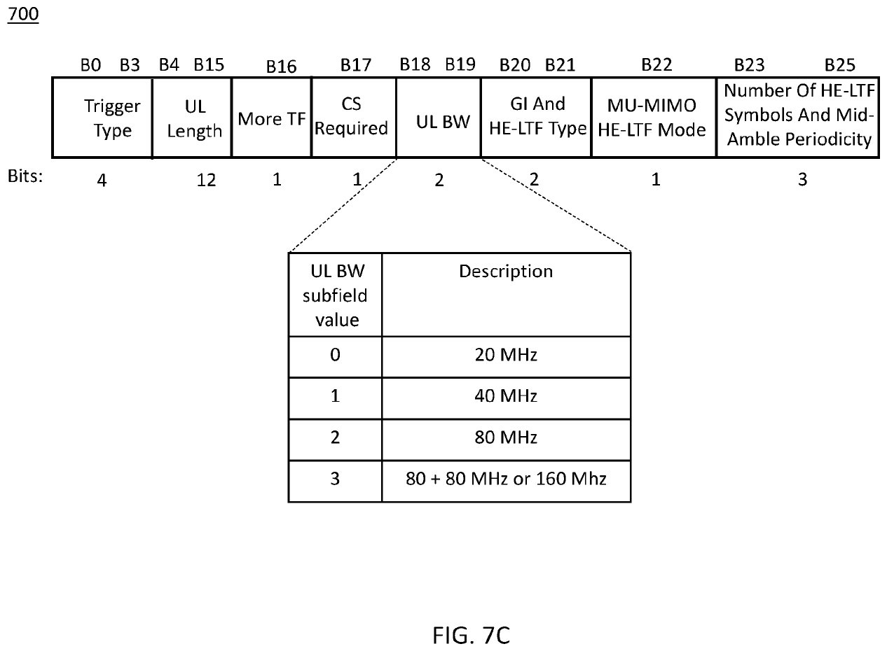

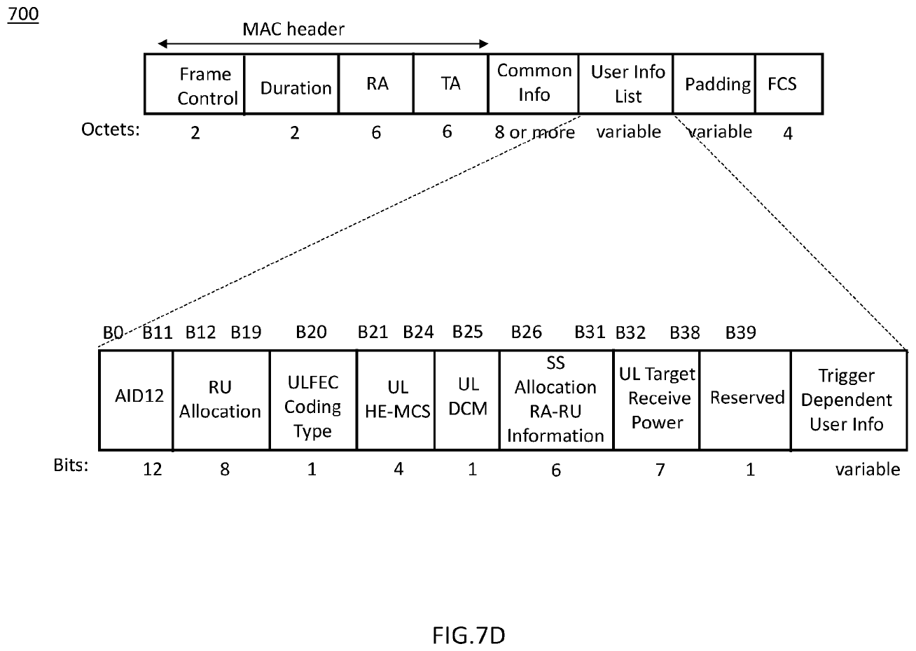

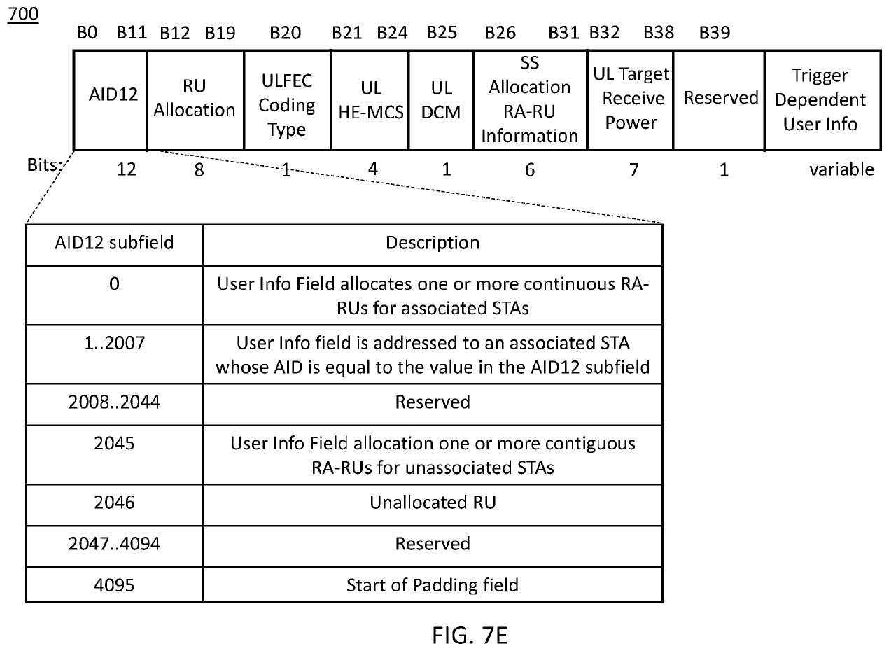

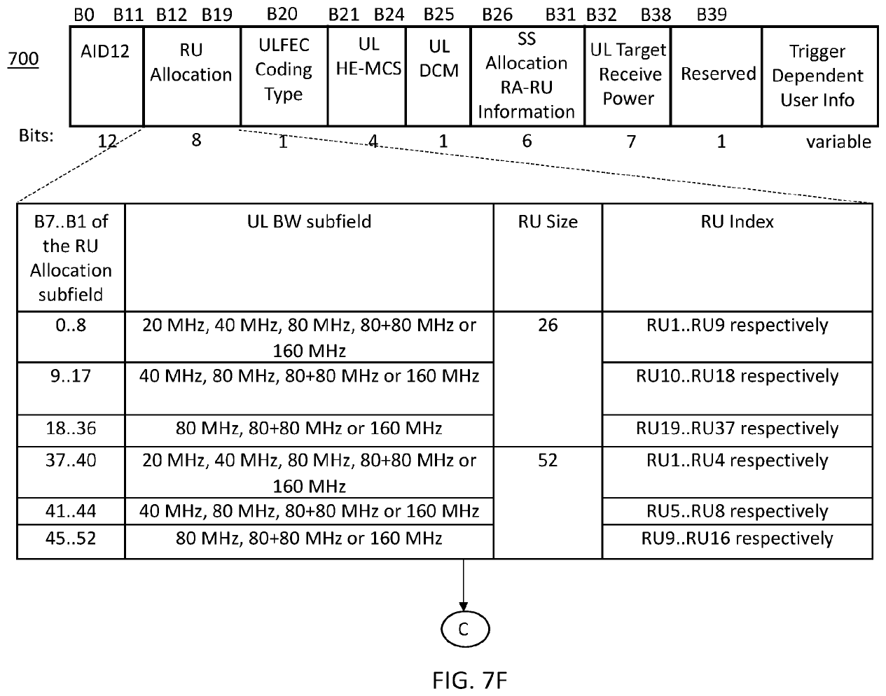

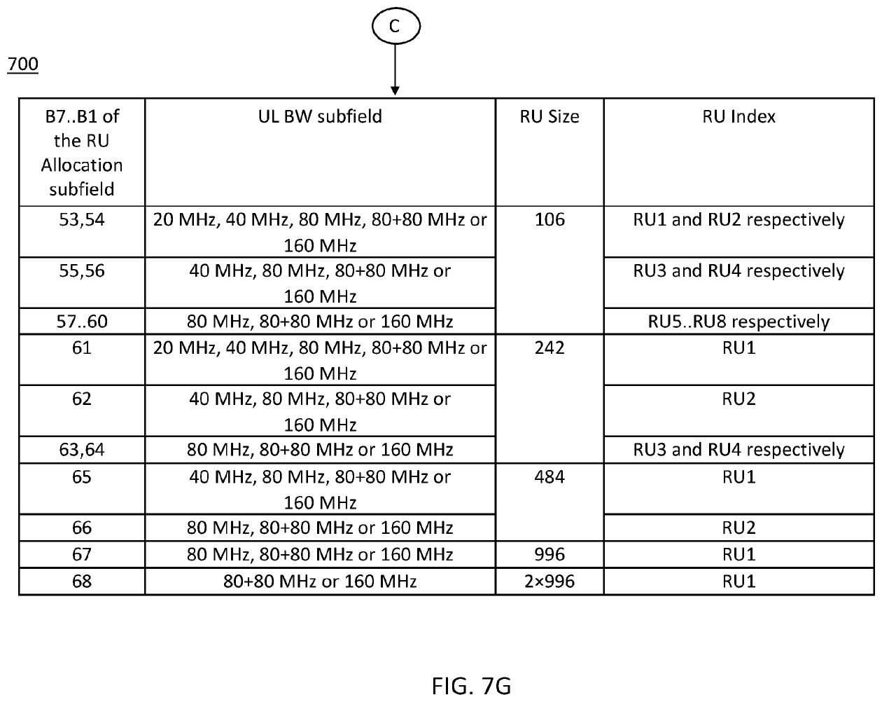

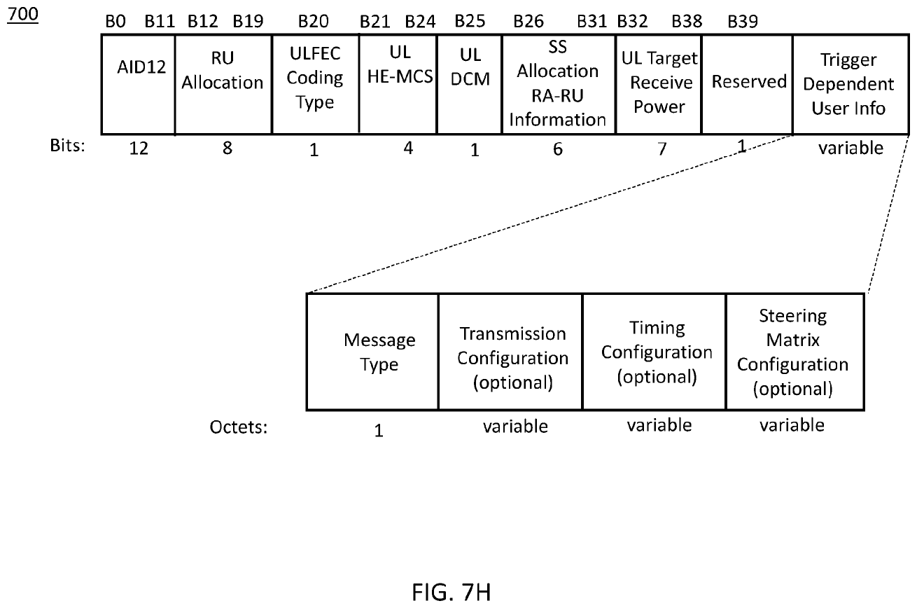

According to an implementation, hierarchy of the fields within a trigger frame is shown in FIG. 7A to FIG. 7H. The trigger frame may interchangeably be referred to as UL-OFDMA sensing trigger message.

As described in FIG. 7A, the Common Info field contains information which is common to plurality of sensing transmitters 504-(1-M). As described in FIG. 7B, a new Trigger Type (bits B0 to B3 of “Common Info” field) may be defined which represents the UL-OFDMA sensing trigger message. The UL-OFDMA sensing trigger message may have a Trigger Type subfield value of 8.

As described in FIG. 7C the UL-OFDMA sensing trigger message may have an uplink bandwidth (UL BW) subfield value of 0, 1, 2 or 4 corresponding to bandwidths of 20 MHz, 40 MHz, 80 MHz, or 80+80 MHz (160 MHz).

As described in FIG. 7D, the User Info List contains information which is specific to each of plurality of sensing transmitters 504-(1-M).

As described in FIG. 7E, the AID12 subfield may be used to address a specific sensing transmitter of plurality of sensing transmitters 504-(1-M).

As described in FIG. 7F and FIG. 7G, the RU Allocation subfield is used to allocate resource units (RU) to each of plurality of sensing transmitters 504-(1-M).

As described in FIG. 7H, the Trigger Dependent User Info subfield may be used to request the transmission configuration and/or steering matrix configuration for each of plurality of sensing transmitters 504-(1-M) that the UL-OFDMA sensing trigger message is triggering.

For ease of explanation and understanding, the description below is provided with reference to sensing transmitter 504-1, however the description is equally applicable to remaining sensing transmitters 504-(2-M).

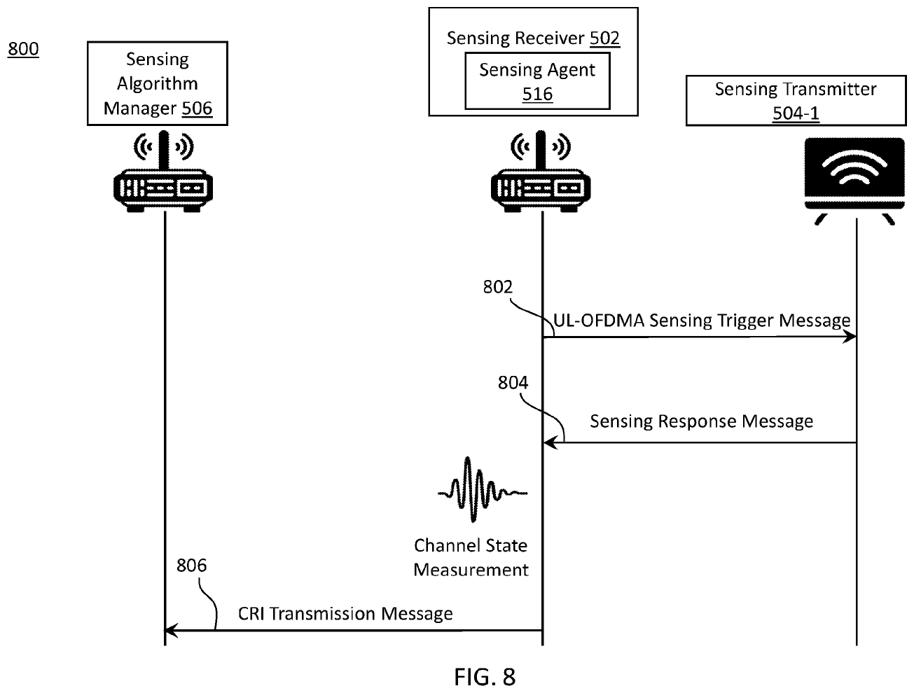

FIG. 8 depicts sequence diagram 800 for communication between sensing receiver 502, sensing transmitter 504-1, and sensing algorithm manager 506, where sensing receiver 502 is a sensing initiator, according to some embodiments. FIG. 8 shows an example of a network (for example, 802.11 network) where sensing algorithm manager 506 is a separate device.

As shown in FIG. 8, at step 802, sensing receiver 502 may initiate a sensing session and send an UL-OFDMA sensing trigger message to sensing transmitter 504-1 requesting a sensing transmission. At step 804, sensing transmitter 504-1 may send a sensing response message as a sensing transmission to sensing receiver 502 in response to the UL-OFDMA sensing trigger message. Upon receiving the sensing response message, sensing receiver 502 may perform a channel state measurement on the received sensing transmission and generate channel representation information (CRI) using the channel representation information configuration. In an example, sensing receiver 502 may generate a reduced CRI. At step 806, sensing receiver 502 may send a CRI transmission message including the channel state measurement (i.e., the reduced CRI) to sensing algorithm manager 506 over the air for further processing. In another example, sensing receiver 502 may generate a reduced filtered CRI. At step 806, sensing receiver 502 may send a CRI transmission message including the channel state measurement (i.e., the reduced filtered CRI) to sensing algorithm manager 506 over the air for further processing.

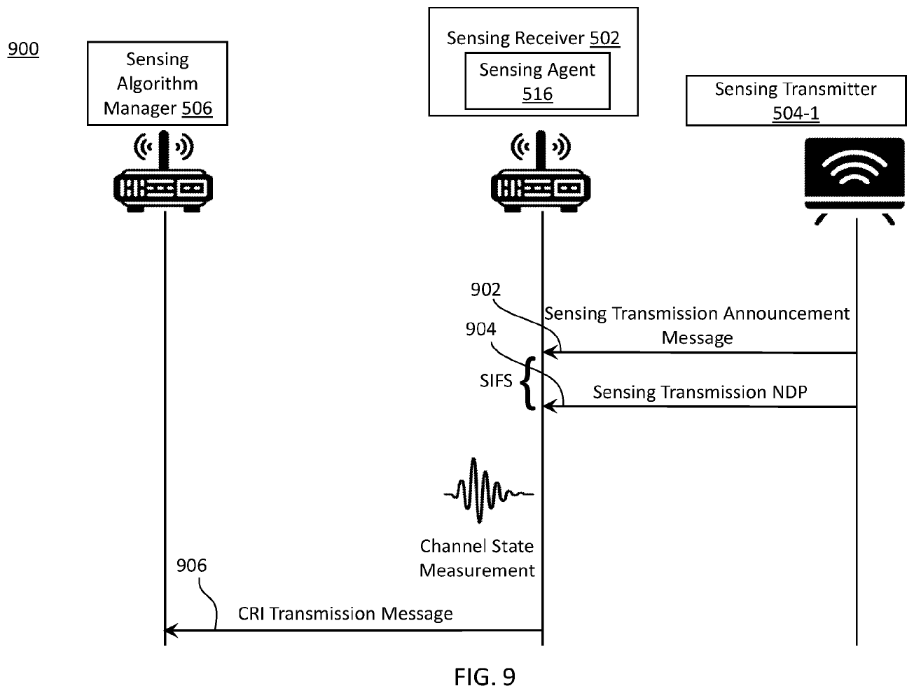

FIG. 9 depicts sequence diagram 900 for communication between sensing receiver 502, sensing transmitter 504-1, and sensing algorithm manager 506, where sensing transmitter 504-1 is a sensing initiator, according to some embodiments. FIG. 9 shows an example of a network (for example, 802.11 network) where sensing algorithm manager 506 is a separate device. As shown in FIG. 9, at step 902, sensing transmitter 504-1 may initiate a sensing session and send a sensing transmission announcement message followed by a sensing transmission NDP to sensing receiver 502. As described in step 904, the sensing transmission NDP follows the sensing transmission announcement message after one SIFS. In an example, the duration of SIFS is 10 μs. Sensing receiver 502 may perform a channel state measurement on the sensing transmission NDP and generate CRI based on the channel representation information configuration. In an example, sensing receiver 502 may generate reduced CRI. At step 906, sensing receiver 502 may send a CRI transmission message including the channel state measurement (i.e., the reduced CRI) to sensing algorithm manager 506 over the air for further processing. In another example, sensing receiver 502 may generate reduced filtered TD-CRI. At step 906, sensing receiver 502 may send a CRI transmission message including the channel state measurement (i.e., the reduced filtered TD-CRI) to sensing algorithm manager 506 over the air for further processing.

As described above, some embodiments of the present disclosure define two sensing message types for Wi-Fi sensing, namely, UL-OFDMA sensing trigger message and sensing response message. In an example, message types are carried in a newly defined IEEE 802.11 Management frame. In some examples, message types are carried in a newly defined IEEE 802.11 Control frame. In some examples, a combination of Management and Control frames may be used to realize these sensing message types. In some examples, timing configuration, transmission configuration, and steering matrix configuration as described in FIG. 7H are implemented as IEEE 802.11 elements.

In one or more embodiments, the sensing message types may be identified by the message type field, and each sensing message type may or may not carry the other identified elements, according to some embodiments. Examples of sensing message types and configuration elements are provided in Table 1.

TABLE 1
Sensing message types and configuration elements

| | Message | Message | Transmission | Timing | Steering Matrix |
|---|---|---|---|---|---|
| Value | Type | Direction | Configuration | Configuration | Configuration |
| 2 | Sensing | Sensing | Optional | Not Required | Optional |
| | trigger | receiver to | Option 1: If | Although this | Option 1: If this |
| | message | sensing | this element is | field may in | element is |
| | | transmitter | absent then | some cases be | absent, then |
| | | | sensing | optional in a | sensing |
| | | | transmitter may | sensing trigger | transmitter |
| | | | use | message, its | transmits the one |
| | | | preconfigured | use is not | or more sensing |
| | | | requested | supported, and | transmissions |
| | | | transmission | it may be | specified by the |
| | | | configuration | omitted. | sensing trigger |
| | | | values. | In examples, if | message using |
| | | | Option 2: If | this | the |
| | | | this element is | configuration | preconfigured |
| | | | present in the | is present then | default steering |
| | | | sensing trigger | it will be | matrix |
| | | | message, | ignored. | configuration. |
| | | | sensing | When this | Option 2: If this |
| | | | transmitter | configuration | element is |
| | | | applies the | is absent, then | present, the |
| | | | requested | this message | element specifies |
| | | | transmission | initiates a | a steering matrix |
| | | | configuration | single sensing | to use for |
| | | | from this | transmission. | sensing |
| | | | element. | | transmitter |
| | | | | | sensing |
| | | | | | transmission, or |
| | | | | | a series of |
| | | | | | steering matrix |
| | | | | | configurations to |
| | | | | | use for sensing |
| | | | | | transmissions of |
| | | | | | a measurement |
| | | | | | campaign. |
| | | | | | The steering |
| | | | | | matrix |
| | | | | | configuration(s) |
| | | | | | can be specified |
| | | | | | using indices |
| | | | | | into a |
| | | | | | preconfigured |
| | | | | | steering matrix |
| | | | | | configuration |
| | | | | | table, or specific |
| | | | | | beamforming |
| | | | | | weights for each |
| | | | | | transmit path or |
| | | | | | transmitting |
| | | | | | antenna of |
| | | | | | sensing |
| | | | | | transmitter may |
| | | | | | be specified. |
| 4 | Sensing | Sensing | Optional | N/A | Optional |
| | response | transmitter | Option 1: | | Option 1: |
| | message | to sensing | Transmission | | Steering matrix |
| | | receiver | parameters of | | configuration |
| | | | this | | applied to this |
| | | | transmission | | transmission. |
| | | | (delivered | | Option 2: Index |
| | | | transmission | | into a |
| | | | configuration) | | preconfigured |
| | | | Option 2: A | | steering matrix |
| | | | single bit flag | | configuration |
| | | | if sensing | | table indicating |
| | | | transmitter | | the steering |
| | | | applies the | | matrix |
| | | | requested | | configuration |
| | | | transmission | | applied to this |
| | | | configuration. | | transmission. |
| | | | Option 3: If | | Option 3: If this |
| | | | this element is | | element is absent |
| | | | absent then | | then sensing |
| | | | sensing | | transmitter |
| | | | transmitter | | applies the |
| | | | applies the | | requested |
| | | | requested | | steering matrix |
| | | | transmission | | configuration |
| | | | parameters | | |
| 0, 1, 3 | Reserved | | N/A | N/A | N/A |
| and | | | | | |
| 5 . . . 255 | | | | | |

Exemplary transmission configuration elements (for example, required transmission configuration or delivered transmission configuration) for a sensing transmission are provided in Table 2.

TABLE 2
Transmission Configuration Element Details

| Name | Type | Valid Range | Description |
|---|---|---|---|
| SensingFrequencyBand | A set of | As defined in Table 3 | Specifies the band in |
| | frequency | (SensingFrequencyBand | which sensing receiver is |
| | band values | details) | to take the sensing |
| | or identifiers | | measurement |
| SensingBandwidth | N/A | N/A | N/A |
| SensingChannel | N/A | N/A | N/A |
| SensingTrainingField | A set of | As defined in Table 4 | Identifies the training |
| | training field | (SensingTrainingField | field which is to be used |
| | values | details) | for the sensing |
| | | | measurement |
| SensingSpatialConf- | Integer | 0 . . . 15 | Index into a table of |
| Index | | | steering matrix |
| | | | configurations, such as |
| | | | may be preconfigured for |
| | | | sensing transmitter via a |
| | | | sensing configuration |
| | | | message and optionally |
| | | | acknowledged by a |
| | | | sensing configuration |
| | | | response message. |
| | | | 0 may be reserved to |
| | | | indicate no configuration |
| | | | requirement (e.g., the |
| | | | sensing transmitter may |
| | | | use a default spatial |
| | | | matrix configuration) and |
| | | | 15 may be reserved to |
| | | | indicate for sensing |
| | | | transmitter to apply the |
| | | | steering matrix |
| | | | configuration specified by |
| | | | the SensingSpatialConf- |
| | | | Index |
| SensingSpatialConfSteeringMatrix | A set of | As defined in Table 5 | A series of steering |
| | spatial | (SensingSpatialConfSteeringMatrix | vectors values (i.e., |
| | steering | details) | spatial matrix |
| | vector | | configurations) which are |
| | values, for | | applied to each of the |
| | example a | | implemented antennas on |
| | phase and | | sensing transmitter prior |
| | gain value, | | to the sending of a |
| | or a real (I) | | sensing transmission |
| | and | | |
| | imaginary | | |
| | (Q) value, | | |
| | each | | |
| | representing | | |
| | a steering | | |
| | matrix | | |
| | configuration | | |

TABLE 3
SensingFrequencyBand details

| Value | Meaning | |
|---|---|---|
| 0 | Reserved | |
| 1 | 2.4 | GHz |
| 2 | 5 | GHz |
| 3 | 6 | GHz |
| 4 | 60 | GHz |

| | |
|---|---|
| 5 . . . 15 | Reserved |

TABLE 4
SensingTrainingField details

| Value | Meaning |
|---|---|
| 0 | Reserved |
| 1 | L-LTF |
| 2 | HT-LTF |
| 3 | VHT-LTF |
| 4 | HE-LTF |
| 5 . . . 15 | Reserved |

TABLE 5
SensingSpatialConfSteeringMatrix details

| | | Valid | |
|---|---|---|---|
| Name | Type | Range | Description |
| TransmissionAntenna- | Integer | 1 . . . 8 | Number of transmission antennas |
| Count | | | on the sensing transmitter used for |
| | | | sensing transmissions. |
| | | | Defines the number of |
| | | | SensingAntennaNSteeringVectorRe |
| | | | and |
| | | | SensingAntennaNSteeringVectorImpairs |
| | | | that follow in the element. At |
| | | | least one antenna must be specified |
| SensingAntenna0- | Half-precision | | Real part of the steering vector for |
| SteeringVectorRe | float (16 bits) | | antenna 0 |
| SensingAntenna0- | Half-precision | | Imaginary part of the steering |
| SteeringVectorIm | float (16 bits) | | vector for antenna 0 |
| . | . | | . |
| . | . | | . |
| . | . | | . |
| SensingAntenna7- | Half-precision | | Real part of the steering vector for |
| SteeringVectorRe | float (16 bits) | | antenna 7 |
| SensingAntenna7- | Half-precision | | Imaginary part of the steering |
| SteeringVectorIm | float (16 bits) | | vector for antenna 7 |

Table 2 describes transmission configuration elements (requested transmission configuration or delivered transmission configuration) for a sensing transmission. In an example, these data are encoded into an element for inclusion in sensing messages between sensing receiver 502 and plurality of sensing transmitters 504-(1-M) or vice versa. In a measurement campaign involving multiple sensing transmitters, these parameters may be defined for all sensing transmitters (i.e., per sensing transmitter). When transmitted from a sensing receiver to a sensing transmitter then these parameters may configure a sensing transmission and when transmitted from the sensing transmitter to the sensing receiver then these parameters may report the configuration used by the sensing transmitter for the sensing transmission.

According to some implementations, the steering matrix configuration element details are described in Table 6.

TABLE 6
Steering Matrix Configuration Element details

| | | Valid | |
|---|---|---|---|
| Name | Type | Range | Description |
| LookupEntriesCount | Integer | 1 . . . 14 | Number of entries in the lookup |
| | | | table specified by this element. |
| | | | Defines the number of EntryM . . . |
| | | | sets of data that follow in the |
| | | | element. At least one entry must be |
| | | | specified. |
| TransmissionAntenna- | Integer | 1 . . . 8 | Number of transmission antennas |
| Count | | | on the sensing transmitters used for |
| | | | sensing transmissions. |
| | | | Defines the number of |
| | | | SensingAntennaNSteeringVectorRe |
| | | | and |
| | | | SensingAntennaNSteeringVectorIm |
| | | | pairs that follow in the element. At |
| | | | least one antenna must be specified. |
| Entry1SensingAntenna0- | Half- | | Real part of the steering vector for |
| SteeringVectorRe | precision | | antenna 0 in lookup table entry 1 |
| | float (16 | | |
| | bits) | | |
| Entry1SensingAntenna0- | Half- | | Imaginary part of the steering |
| SteeringVectorIm | precision | | vector for antenna 0 in lookup table |
| | float (16 | | entry 1 |
| | bits) | | |
| . | . | | . |
| . | . | | . |
| . | . | | . |
| Entry1SensingAntenna7- | Half- | | Real part of the steering vector for |
| SteeringVectorRe | precision | | antenna 7 in lookup table entry 1 |
| | float (16 | | |
| | bits) | | |
| Entry1SensingAntenna7- | Half- | | Imaginary part of the steering |
| SteeringVectorIm | precision | | vector for antenna 7 in lookup table |
| | float (16 | | entry 1 |
| | bits) | | |
| . | . | | . |
| . | . | | . |
| . | . | | . |
| Entry14SensingAntenna0- | Half- | | Real part of the steering vector for |
| SteeringVectorRe | precision | | antenna 0 in lookup table entry 14 |
| | float (16 | | |
| | bits) | | |
| Entry14SensingAntenna0- | Half- | | Imaginary part of the steering |
| SteeringVectorIm | precision | | vector for antenna 0 in lookup table |
| | float (16 | | entry 14 |
| | bits) | | |
| . | . | | . |
| . | . | | . |
| . | . | | . |
| Entry14SensingAntenna7- | Half- | | Real part of the steering vector for |
| SteeringVectorRe | precision | | antenna 7 in lookup table entry 14 |
| | float (16 | | |
| | bits) | | |
| Entry14SensingAntenna7- | Half- | | Imaginary part of the steering |
| SteeringVectorIm | precision | | vector for antenna 7 in lookup table |
| | float (16 | | entry 14 |
| | bits) | | |

In an example, the data provided in Table 6 may be encoded into an element for inclusion in the messages between sensing receiver 502 and plurality of sensing transmitters 504-(1-M). In a measurement campaign involving multiple sensing transmitters, these parameters may be defined for all devices. When transmitted from sensing receiver 502 to plurality of sensing transmitters 504-(1-M), then the steering matrix configurations populate a lookup table (which can later be accessed via an index).

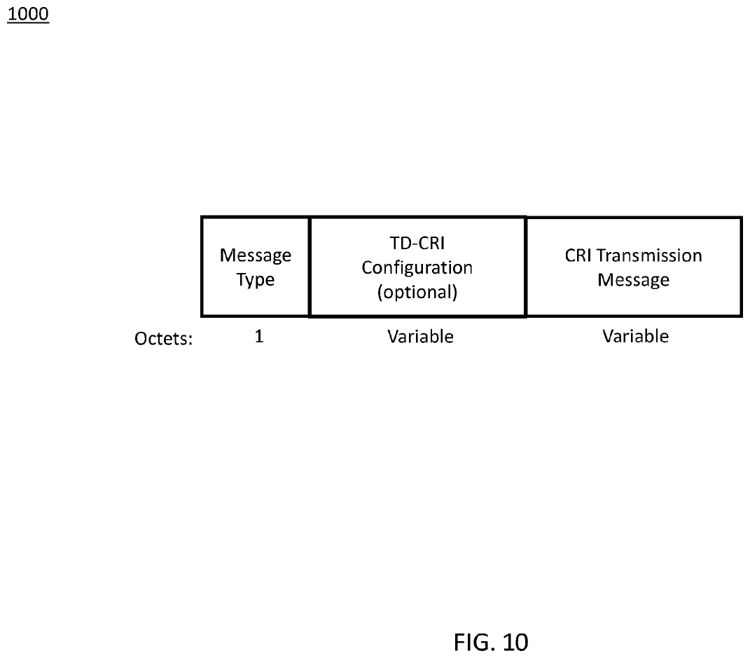

According to some implementations, when sensing receiver 502 has calculated a sensing measurement and created channel representation information (for example, in form of reduced filtered CRI), the sensing receiver 502 may be required to communicate the channel representation information to sensing algorithm manager 506. In the examples, the reduced filtered CRI may be transferred by a management frame. In an example, a message type may be defined which represents a CRI Transmission Message. FIG. 10 illustrates an example of a component of management frame 1200 carrying a CRI transmission message, according to some embodiments. In an example, system 500 may require acknowledgement frames and the management frame carrying the CRI transmission message may be implemented as an Action frame and in another example, system 500 may not require acknowledgement frames and the management frame carrying the CRI transmission message may be implemented as an Action No Ack frame. Examples of CRI transmission message and TD-CRI configuration elements are shown in Table 7. Further, the CRI transmission message element details are shown in Table 8.

TABLE 7
CRI Transmission Message and TD-CRI Configuration Elements

| | Message | Message | TD-CRI | |
|---|---|---|---|---|
| Value | Type | Direction | Configuration | CRI Transmission Message |
| 6 | CRI | Sensing | Optional | CRI Transmission Message as |
| | Transmission | receiver to | In the CRI | defined in Table 8 |
| | Message | sensing | transmission sent | |
| | | algorithm | by the sensing | |
| | | manager | receiver, this | |
| | | | element is present | |
| | | | if reduced filtered | |
| | | | TD-CRI are | |
| | | | being sent and | |
| | | | absent if CSI are | |
| | | | being sent. | |
| | | | If this element is | |
| | | | present, the TD- | |
| | | | CRI | |
| | | | configuration that | |
| | | | was used to | |
| | | | create the reduced | |
| | | | filtered TD-CRI | |
| | | | may be sent along | |
| | | | with the reduced | |
| | | | filtered TD-CRI | |
| | | | values. | |
| | | | In another | |
| | | | example, if this | |
| | | | element is | |
| | | | present, the | |
| | | | element may | |
| | | | contain a bit flag | |
| | | | to indicate that | |
| | | | the sensing | |
| | | | receiver applied | |
| | | | the TD-CRI | |
| | | | configuration that | |
| | | | was in the | |
| | | | sensing | |
| | | | transmission | |
| | | | announcement | |
| | | | message which | |
| | | | triggered the | |
| | | | response. | |
| | | | Alternatively, the | |
| | | | element may | |
| | | | contain multiple | |
| | | | bit flags, one for | |
| | | | each aspect of the | |
| | | | TD-CRI | |
| | | | configuration, to | |
| | | | indicate which of | |
| | | | the aspects of the | |
| | | | TD-CRI | |
| | | | configuration | |
| | | | were applied. | |

TABLE 8
CRI Transmission Message Element Details

| Name | Type | Valid Range | Description |
|---|---|---|---|
| IFFT Length | Integer | NFFT | Specifies the length of the |
| | | | IFFT used to calculate the |
| | | | full TD-CRI from the CSI. |
| | | | This value describes the |
| | | | maximum number of active |
| | | | time-domain pulses which |
| | | | may be returned in the |
| | | | reduced filtered TD-CRI |
| | | | and therefore the length of |
| | | | the bit field that represents |
| | | | the active time domain |
| | | | pulses. |
| Active Frequency | Bit field | Length of NFFT | Specifies the position in the |
| Subcarriers | | with 1 representing | CSI of a subcarrier |
| | | an active subcarrier | associated with a sensing |
| | | in the frequency | transmission from the |
| | | domain | sensing transmitter. (A zero |
| | | | in the bit field indicates that |
| | | | the corresponding |
| | | | subcarrier is part of an |
| | | | isolating band.) |
| Active Time | Bit field | Length of NFFT | Specifies the position in the |
| Domain Pulses | | with 1 representing | full TD-CRI of an active |
| | | an active time- | time domain pulse. A value |
| | | domain pulse | of 0 means that a tone is |
| | | | vacant and a value of 1 |
| | | | means that a tone is |
| | | | occupied. There follows in |
| | | | this element exactly one |
| | | | magnitude value and one |
| | | | phase value for each value |
| | | | of 1 in the bit field. |
| CRI Maximum | Unsigned | [0.0, 1.0] | Maximum magnitude value |
| Magnitude 1 | Float | | for the first active time |
| | | | domain pulse. |
| CRI Minimum | Unsigned | [0.0, 1.0] | Minimum magnitude value |
| Magnitude 1 | Float | | for the first active time |
| | | | domain pulse. |
| CRI Phase 1 | Signed | [−π/2, π/2) | Phase value for the first |
| | Float | | active time domain pulse. |
| . | . | . | . |
| . | . | . | . |
| . | . | . | . |
| CRI Maximum | Unsigned | [0.0, 1.0] | Maximum magnitude value |
| Magnitude m | Float | | for the mth active time |
| | | | domain pulse. |
| CRI Minimum | Unsigned | [0.0, 1.0] | Minimum magnitude value |
| Magnitude m | Float | | for the mth active time |
| | | | domain pulse. |
| CRI Phase m | Signed | [−π/2, π/2) | Phase value for the mth |
| | Float | | active time domain pulse. |

Table 8 shows an example of a CRI transmission message element which transfers the TD-CRI using a bit field to represent the active (included/selected) time domain pulses. In an example, the data structure described in Table 8 may be used to format the reduced filtered TD CRI data. In an example, a proprietary header or descriptor may be added to the data structure to allow sensing algorithm manager 506 to detect that the data structure is of the form of a CRI transmission message element. In an example, data may be transferred in the format shown in FIG. 10 and sensing algorithm manager 506 may be configured to interpret the Message Type value that represents a CRI Transmission Message.

### C. Systems and Methods for Compressed Channel State Information for Virtual Wideband Channels

The present disclosure generally relates to systems and methods for Wi-Fi sensing. In particular, the present disclosure relates to systems and methods for compressed channel state information (CSI) for virtual wideband channels.

According to one or more implementations, wideband channels which include 40 MHz wideband channel, 80 MHz wideband channel, and 160 MHz wideband channel are specified and supported by IEEE 802.11 standard. Other bandwidths which are not specified and supported by IEEE 802.11 standard but may be specified and supported at a future time may also be represented by the definition, wideband channel. Each of the 40 MHz wideband channel, 80 MHz wideband channel, and 160 MHz wideband channel respectively occupies two, four, and eight contiguous 20 MHz component frequency bands. However, creating wideband channels in this way is possible only when multiple contiguous 20 MHz component frequency bands are available. There may be scenarios where no sufficient contiguous 20 MHz component frequency bands are available to constitute a wideband channel.

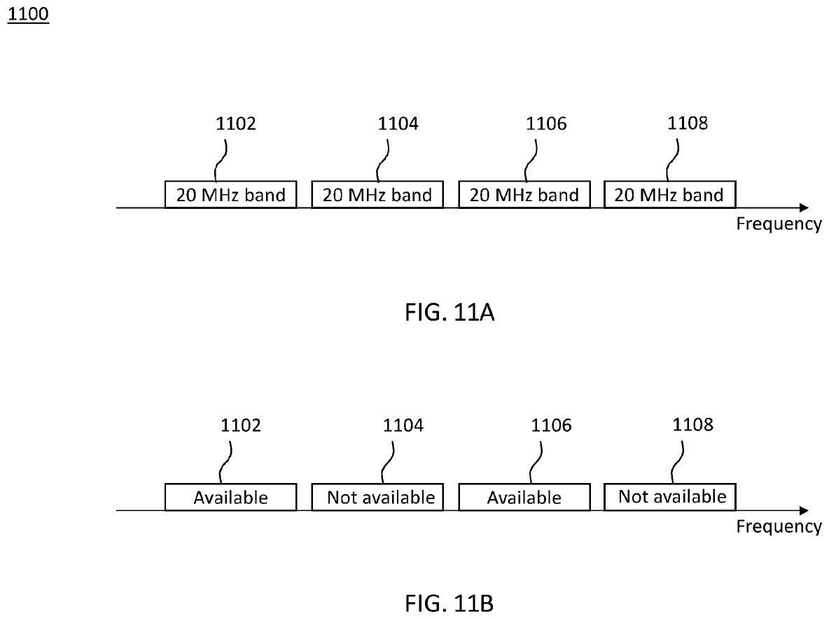

FIG. 11A and FIG. 11B depict four 20 MHz component frequency bands and their availability status, according to some embodiments. FIG. 11A depicts four 20 component frequency bands, namely, first component frequency band 1102, second component frequency band 1104, third component frequency band 1106, and fourth component frequency band 1108. FIG. 11B depicts that only first component frequency band 1102 and third component frequency band 1106 are available for use, while second component frequency band 1104 and fourth component frequency band 1108 are not available. In an example, second component frequency band 1104 and fourth component frequency band 1108 may be in use by other devices. As described in FIG. 11A, first component frequency band 1102 and second component frequency band 1104 are non-contiguous component frequency bands. Further, although first component frequency band 1102 and third component frequency band 1106 are available, first component frequency band 1102 and third component frequency band 1106 are isolated (or separated) by second component frequency band 1104. In an implementation, a 40 MHz wideband channel may be formed by concatenating first component frequency band 1102 and third component frequency band 1106 (even though first component frequency band 1102 and third component frequency band 1106 are isolated by second component frequency band 1104) as a whole entity. This type of a wideband channel, made up of one or more non-contiguous component bands, is called a virtual wideband channel.

The virtual wideband channel made up by first component frequency band 1102 and third component frequency band 1106 is a 40 MHz virtual wideband. First component frequency band 1102 and third component frequency band 1106 may be referred to as the component bands of the virtual wideband while second component frequency band 1104 may be referred to as an isolated frequency band. A virtual wideband may include multiple component frequency bands, and each of the component frequency bands may be contiguous or may be non-contiguous. Where two component frequency bands are non-contiguous, the isolated frequency band that separates the two component frequency bands from each other may be unused or may potentially be assigned to a different device. In some implementations, component frequency bands assigned to a sensing transmitter may or may not be separated by one or more isolated frequency bands.

While the example shown in FIG. 11A and FIG. 11B illustrates that each of the component frequency bands are 20 MHz in bandwidth, in some embodiments, each component frequency band may have a different bandwidth. In an example, the amount of bandwidth (in particular in the case where the bandwidth is variable) may be expressed in terms of “Resource Units (RUS)”. Each RU is made up of a numbers of subcarriers. Subcarriers may also be referred to as tones.

Referring again to FIG. 5, according to one or more implementations, uplink orthogonal frequency division multiple access (UL-OFDMA) as defined by IEEE 802.11ax may be used to assign bandwidth for plurality of sensing transmitters 504-(1-M) to make uplink (i.e., from a sensing transmitter to a sensing receiver) sensing transmissions. According to an implementation, sensing receiver 502 may secure a TXOP which may be allocated to the uplink sensing transmissions by plurality of sensing transmitters 504-(1-M). In an implementation, sensing receiver 502 may assign multiple component frequency bands of a wideband signal in the secured TXOP to plurality of sensing transmitters 504-(1-M) for simultaneous uplink sensing transmissions.

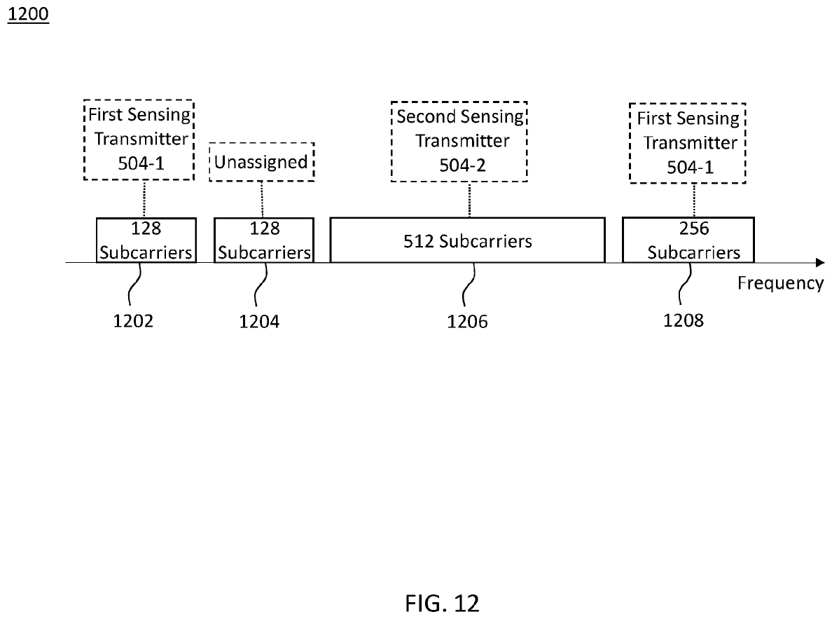

FIG. 12 depicts an example of a virtual wideband for a multi-user uplink sensing transmission using UL-OFDMA, according to some embodiments. In the example, the complete uplink sensing transmission received by sensing receiver 502 may include three component frequency bands assigned to first sensing transmitter 504-1 and second sensing transmitter 504-2 and one unassigned component frequency band. Each of the assigned component frequency bands has a different bandwidth. As described in FIG. 12, component frequency band 1202 with 128 subcarriers and component frequency band 1208 with 256 subcarriers are assigned to first sensing transmitter 504-1. Further, single component frequency band 1206 with 512 subcarriers is assigned to second sensing transmitter 504-2. Also, component frequency band 1204 with 128 subcarriers is unassigned. From the perspective of first sensing transmitter 504-1, component frequency band 1202 and component frequency band 1208 are separated by 640 isolated subcarriers (i.e., subcarriers of an isolated band). In the example of FIG. 12, component bands assigned to the same sensing transmitter (for example, component frequency band 1202 and component frequency band 1208 assigned to first sensing transmitter 504-1) may form a virtual wideband. Accordingly, the virtual wideband of first sensing transmitter 504-1 includes 384 subcarriers.

Referring to FIG. 5, according to one or more implementations, for the purpose of Wi-Fi sensing, sensing receiver 502 may initiate a measurement campaign (or a Wi-Fi sensing session). In the measurement campaign, exchange of transmissions between sensing receiver 502 and plurality of sensing transmitter 504-(1-M) may occur. In an example, control of these transmissions may be by the MAC layer of the IEEE 802.11 stack.

According to an example implementation, sensing receiver 502 may initiate the measurement campaign via one or more sensing trigger messages. In an implementation, sensing agent 516 may be configured to generate a sensing trigger message configured to trigger a series of sensing transmissions from plurality of sensing transmitters 504-(1-M). According to an implementation, sensing receiver 502 may secure a TXOP which may be allocated by sensing receiver 502 to the series of sensing transmissions by plurality of sensing transmitters 504-(1-M). The series of sensing transmissions may include a sensing transmission from each of plurality of sensing transmitters 504-(1-M). In an example, the sensing trigger message may be an UL-OFDMA sensing trigger message which may instruct plurality of sensing transmitters 504-(1-M) to make sensing transmissions using UL-OFDMA. In an example, the sensing trigger message may include a requested transmission configuration field. Other examples of information/data included in the sensing trigger message that are not discussed here are contemplated herein.

According to an implementation, sensing agent 516 may transmit the sensing trigger message to plurality of sensing transmitters 504-(1-M). In an implementation, sensing agent 516 may transmit the sensing trigger message to plurality of sensing transmitters 504-(1-M) via transmitting antenna 512 to trigger the series of sensing transmissions from plurality of sensing transmitters 504-(1-M).

In response to receiving the sensing trigger message, each of plurality of sensing transmitters 504-(1-M) may generate a sensing transmission. In an example, the sensing transmission that the sensing trigger message triggers from each of plurality of sensing transmitters 504-(1-M) may be a sensing response message. In an implementation, each of plurality of sensing transmitters 504-(1-M) may generate a sensing transmission using the requested transmission configuration. In an implementation, plurality of sensing transmitters 504-(1-M) may make the sensing transmissions in a single TXOP. According to an implementation, each of plurality of sensing transmitters 504-(1-M) may transmit respective sensing transmission to sensing receiver 502 in response to the sensing trigger message and in accordance with the requested transmission configuration. In an example, each sensing transmission may include a delivered transmission configuration corresponding to the transmission configuration used to deliver the sensing transmission. In an example, when it may be supported by the sensing transmitter the delivered transmission configuration corresponds to the requested transmission configuration.

In an implementation, sensing receiver 502 may receive the sensing transmissions from plurality of sensing transmitters 504-(1-M) transmitted in response to the sensing trigger message. Sensing receiver 502 may be configured to receive the sensing transmissions from plurality of sensing transmitters 504-(1-M) via receiving antenna 514. According to an implementation, sensing agent 516 may be configured to generate a sensing measurement representing a channel state information (CSI) based on the sensing transmissions.

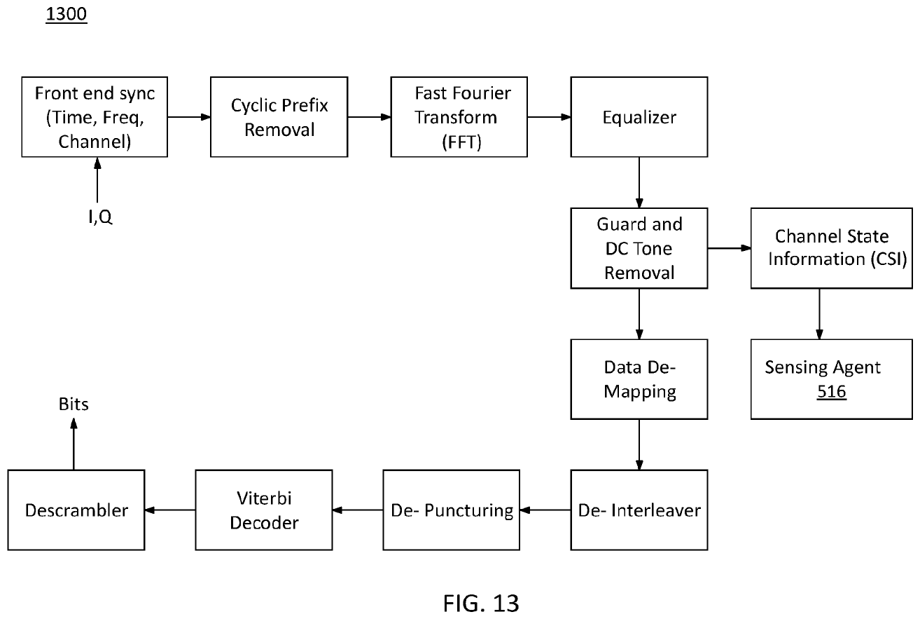

According to an implementation, a baseband receiver of sensing receiver 502 may be configured to calculate the CSI based on the sensing transmissions. In some implementations, sensing receiver 502 may calculate a contribution to the CSI by a receiver chain. In an example, the receiver chain of sensing receiver 502 may include analog elements and digital elements. For example, the receiver chain may include the analog and digital components through which a received signal may travel from a reference point to a point at which the received signal may be read, i.e., by sensing agent 516 of sensing receiver 502. A representation 1300 of the receiver chain of sensing receiver 502 is illustrated in FIG. 13. As described in FIG. 13, In-phase (I) and Quadra phase (Q) modulated symbols arrive at a frond end of the receiver where synchronization is performed including frequency and timing recovery. Further, time domain guard period (cyclic prefix) is removed, and the receiver performs a discrete Fourier transform (DFT) on the received signal (for example, the I and Q modulated symbols). Guard tones and DC tones are then removed. CSI is then generated prior to data de-mapping, de-interleaving (using a de-interleaver), de-puncturing, decoding (using a Viterbi decoder) and finally descrambling (using a descrambler). As a result of descrambling, data bits are generated. The generated CSI is provided to sensing agent 516.

In some implementations, an automatic gain control (AGC) may precondition the I and Q samples prior to digitization. The AGC is a dynamic process, and its gain may change over time depending on conditions in the propagation channel. In some examples, a value of gain applied to the signal may be fed from the AGC processing to allow for a compensation operation.

In an example, sensing receiver 502 may receive an 80 MHz OFDMA signal. The 80 MHz OFDMA signal may include the sensing transmissions from plurality of sensing transmitters 504-(1-M). In the example, the baseband receiver may calculate the CSI on the entire 80 MHz received bandwidth. According to an example, the 80 MHz OFDMA signal may comprise 1024 subcarriers and the baseband receiver may generate 1024 frequency-domain CSI values. According to an implementation, the number of subcarriers in a bandwidth and the number of CSI values generated by the baseband receiver varies according to the total receive bandwidth and to the version of the IEEE 802.11 standard that is used.

According to an implementation, upon receiving the CSI, sensing agent 516 may generate a full time-domain channel representation information (TD-CRI) of the CSI. In an implementation, sensing agent 516 may convert the frequency domain CSI into the full TD-CRI by performing an IDFT on the CSI. In the example, for 1024 CSI values, sensing agent 516 may convert the frequency domain CSI into the full TD-CRI by applying a 1024-point IDFT to the 1024 CSI values. In an implementation, to reduce the amount of CRI that needs to be transmitted over the air, the CRI may be represented by the TD-CRI instead of by the CSI. While CSI provides information of the channel's frequency response (i.e., magnitude attenuation and phase rotation on a signal) at each subcarrier, full TD-CRI may provide the channel's impulse response (i.e., magnitude attenuation and phase rotation of each propagation path delay in a multi-path propagation environment).

In time domain, a propagation channel may be described by a transfer function. In an example, the transfer function may be referred to as h(t). The transfer function may also be described as an impulse response of the propagation channel. The impulse response of the propagation channel may include a plurality of time domain pulses. The plurality of time domain pulses may represent reflections that transmitted signals (for example, those transmitted by a transmitter) underwent before reaching a receiver. A reflected time domain pulse may be represented as:

$$h ( {t}_{k} ) = {α}_{k} δ ( t - {t}_{k} ) \qquad ( 8 )$$

where tₖ represents a time taken by the reflected time domain pulse to reach the receiver by following a discrete reflective path and αₖ represents an attenuation experienced by the reflected time domain pulse between the transmitter and the receiver.

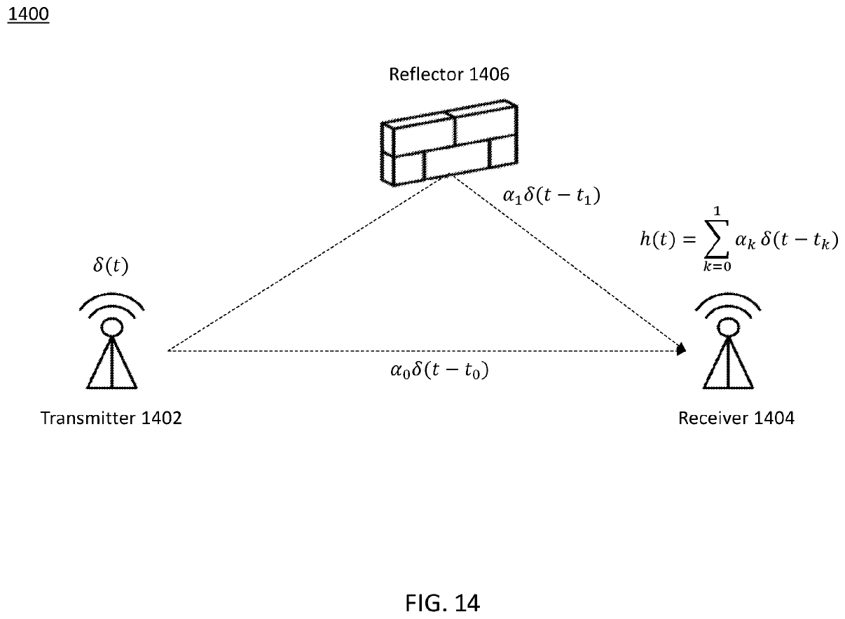

FIG. 14 depicts example representation 1400 of a channel, which includes a direct signal path and a single multipath, according to some embodiments. In an implementation, FIG. 14 depicts discrete multipaths of a time domain pulse, δ(t), between transmitter 1402 and receiver 1404 according to some embodiments. In FIG. 14, a direct path signal is represented as:

$$h ( {t}_{0} ) = {α}_{0} δ ( t - {t}_{0} ) \qquad ( 9 )$$

and a first reflected time domain pulse is represented as:

$$h ( {t}_{1} ) = {α}_{1} δ ( t - {t}_{1} ) \qquad ( 10 )$$

In an implementation, if a number of discrete multipaths is given by Lₚ, then the impulse response of the propagation channel may be represented as:

$$h ( t ) = {\sum }_{k = 0}^{{L}_{p}} {α}_{k} δ ( t - {t}_{k} ) \qquad ( 11 )$$

A time domain representation may be converted to a frequency domain representation using a Fourier transform. In an example, the frequency domain representation of the impulse response of the propagation channel may be given by equation (12):

$$H ( f ) = {\sum }_{k = 0}^{{L}_{p}} {α}_{k} {e}^{- j 2 {π ft}_{k}} \qquad ( 12 )$$

Each value of H(f) in equation (12) may be a linear combination of all values of h(t) in equation (11). In an implementation, the equation (12) may be represented using matrix vector multiplication according to equation (13), provided below.

$${A}_{F , N} = [ \begin{matrix}{e}^{- j 2 π {f}_{0} {t}_{0}} & {e}^{- j 2 π {f}_{0} {t}_{1}} & … & {e}^{- j 2 π {f}_{0} {t}_{{L}_{p}}} \\ {e}^{- j 2 π {f}_{1} {t}_{0}} & {e}^{- j 2 π {f}_{1} {t}_{1}} & … & {e}^{- j 2 π {f}_{1} {t}_{{L}_{p}}} \\ ⋮ & & … & ⋮ \\ {e}^{- j 2 π {f}_{N - 1} {t}_{0}} & … & … & {e}^{- j 2 π {f}_{N - 1} {t}_{{L}_{p}}}\end{matrix} ] \qquad ( 13 )$$

where, A_{F,N} is a Fourier basis matrix of dimension N×(Lₚ+1) and N is the number of frequencies over which the Fourier transform is calculated.

The CSI (H(f)) representation of equation (12) may be expressed in a matrix form according to equation (13). Further, a matrix equation for determining H(f) is shown in equation (14) and equation (15), provided below.

$$[ \text{} \begin{matrix}{e}^{- j 2 π {f}_{0} {t}_{0}} & {e}^{- j 2 π {f}_{0} {t}_{1}} & … & {e}^{- j 2 π {f}_{0} {t}_{{L}_{p}}} \\ {e}^{- j 2 π {f}_{1} {t}_{0}} & {e}^{- j 2 π {f}_{1} {t}_{1}} & … & {e}^{- j 2 π {f}_{1} {t}_{{L}_{p}}} \\ ⋮ & & … & ⋮ \\ {e}^{- j 2 π {f}_{N - 1} {t}_{0}} & … & … & {e}^{- j 2 π {f}_{N - 1} {t}_{{L}_{p}}}\end{matrix} ] \times \text{} \text{} [ \text{} \begin{matrix}{α}_{0} \\ {α}_{1} \\ ⋮ \\ {α}_{{L}_{p}}\end{matrix} ] \text{} = \text{} [ \text{} \begin{matrix}H ( {f}_{0} ) \\ H ( {f}_{1} ) \\ ⋮ \\ H ( {f}_{N - 1} )\end{matrix} ] \qquad ( 14 )$$

$${A}_{F , N} \times α = H ( f ) \qquad ( 15 )$$

In an example, each column of A_{F,N} corresponds to a time domain pulse of h(t). Accordingly, the columns of A_{F,N} are set of all possible tₖ from equation (9). The columns of A_{F,N} together with the column vector, a, are the TD-CRI corresponding to the CSI. In an implementation, the CSI (H(f)) may be represented as time domain pulses.

According to an implementation, sensing agent 516 may generate the full TD-CRI of the CSI by performing IDFT on the CSI (H(f)). When TD-CRI is generated by taking the IDFT of the CSI, H(f), there is a one-to-one correspondence between a frequency domain tone (a complex value of the CSI) and a time domain tone (a complex value of the TD-CRI), and it is referred to as full TD-CRI. The full TD-CRI and CSI form a pair of DFTs. Accordingly, the CSI and the TD-CRI are represented in a Fourier matrix form. In an example, by considering full TD-CRI as a time-domain sequence h and CSI as a frequency-domain sequence H, the full TD-CRI can be derived as the IDFT of a known CSI using equation (16), provided below.

$$H = {B}_{N} h \qquad ( 16 )$$

where B_{N}={b_{n,k}} is the N×N IDFT matrix, whose element at n^{th} row and k^{th} column is:

$${b}_{n , k} = \frac{1}{\sqrt{N}} {e}^{j 2 π k n / N} \qquad ( 17 )$$

The n^{th} row of By corresponds to hₙ of h, the k^{th} column row of B_{N} corresponds to Hₖ of H, and b_{n,k} represents the contribution of Hₖ on hₙ.

In some embodiments, the CSI may be reconstructed as the DFT of a known full TD-CRI using equation (18), provided below.

$$H = {A}_{N} h , \\ where , \qquad ( 18 )$$

$$h = {[ {h}_{0} , {h}_{1} , {h}_{2} , . . . {h}_{N - 1} ]}^{T} \qquad ( 19 )$$

$$H = {[ {H}_{0} , {H}_{1} , {H}_{2} , . . . {H}_{N - 1} ]}^{T} \qquad ( 20 )$$

In an example, hi and Hi represent complex numbers, T represents matrix transpose, N represents a number of DFT points (i.e., DFT size), and A_{N}={αₖ,n} is the N×N DFT matrix, whose element at k^{th} row and n^{th} column is:

$${a}_{k , n} = \frac{1}{\sqrt{N}} {e}^{- j 2 π kn / N} \qquad ( 21 )$$

where, k and n are the frequency and time indices, respectively. In DFT, k=0, 1, . . . , N−1 and n=0, 1, . . . , N−1. Further, k^{th} row of A_{N} corresponds to Hₖ of H in equation (20), the n^{th} column of A_{N} corresponds to hₙ of h in equation (19), and αₖ,n represents the contribution of hₙ on Hₖ.

In equation (16) and equation (18), the subscript of A_{N} and B_{N} indicates that the size of the matrices is N×N. In an example, equation (16) may be used to obtain h when His known, while equation (18) may be used to obtain H when h is known. Alternatively, the equation (16) and the equation (18) may be expressed as equations (22) and (23), respectively, provided below.

$${H}^{T} = {h}^{T} {A}_{N}^{T} \qquad ( 22 )$$

$${h}^{T} = {H}^{T} {B}_{N}^{T} \qquad ( 23 )$$

where the superscript T stands for the matrix transpose.

In an implementation, sensing agent 516 may create a column vector α′ of dimension 1×N comprising amplitudes of each of the TD-CRI values, where N is the number of points in the IDFT. In an example, if N=1024, then the column vector α′ has dimensions of 1×1024. In an example implementation, the column vector α′ is represented using equation (24):

$${α}^{′} = [ \begin{matrix}{α}_{0} \\ {α}_{1} \\ ⋮ \\ {α}_{N - 1}\end{matrix} ] \qquad ( 24 )$$

According to an implementation, sensing agent 516 may remove any an from the column vector α′ whose value is equal to zero or below a predefined threshold. In an example, sensing agent 516 may use other criteria to remove any an from the column vector α′ and to simplify subsequent processing. Sensing agent 516 may retain the information of the position in the column vector α′ for which an was removed. In an example implementation, sensing agent 516 may create a bit field that is N=1024 bits long. Sensing agent 516 may place a zero (0) at each of the locations (starting from the 0th and increasing in order to the N−1th) from where an was removed. Further, sensing agent 516 may place a one (1) at all other locations. The bit field created by sensing agent 516 may be referred to as time domain (TD) bit map. In an example, the number of ones (referred to as the bit weight of the bit map) in the TD bit map is k. The retained, k values may be renumbered from 0 to k−1 and placed consecutively into a new column vector α. In an example implementation, the new column vector α may be represented using equation (25):

$$α = [ \begin{matrix}{α}_{0} \\ {α}_{1} \\ ⋮ \\ {α}_{k - 1}\end{matrix} ] \qquad ( 25 )$$

In an example, the TD bit map may represent active subcarriers in the frequency domain response and guard subcarriers and DC (direct current) subcarriers are not represented. The term active tone TD bit map may describe a TD bit map that represents active subcarriers in the frequency domain response. In another example, the TD bit map may represent all subcarriers in the frequency domain response and guard subcarriers and DC subcarriers are represented by a zero in the TD bit map. The term full tone TD bit map may describe a TD bit map that represents all subcarriers in the frequency domain response.

According to an implementation, sensing agent 516 may create an N×N matrix A_{F,N} represented using equation (26), provided below.

$${A}_{F , N} = [ \begin{matrix}{e}^{- j 2 π {f}_{0} {t}_{0}} & {e}^{- j 2 π {f}_{0} {t}_{1}} & … & {e}^{- j 2 π {f}_{0} {t}_{N - 1}} \\ {e}^{- j 2 π {f}_{1} {t}_{0}} & {e}^{- j 2 π {f}_{1} {t}_{1}} & … & {e}^{- j 2 π {f}_{1} {t}_{N - 1}} \\ ⋮ & & & ⋮ \\ {e}^{- j 2 π {f}_{N - 1} {t}_{0}} & … & … & {e}^{- j 2 π {f}_{N - 1} {t}_{N - 1}}\end{matrix} ] \qquad ( 26 )$$

where, each TD-CRI is arranged in a column of A_{F,N}. For example, column

$$[ \begin{matrix}{e}^{- j 2 π {f}_{0} {t}_{0}} \\ {e}^{- j 2 π {f}_{1} {t}_{0}} \\ ⋮ \\ {e}^{- j 2 π {f}_{N - 1} {t}_{0}}\end{matrix} ]$$

represents the value of TD-CRI 0, column

$$[ \begin{matrix}{e}^{- j 2 π {f}_{0} {t}_{1}} \\ {e}^{- j 2 π {f}_{1} {t}_{1}} \\ ⋮ \\ {e}^{- j 2 π {f}_{N - 1} {t}_{1}}\end{matrix} ]$$

represents the value of TD-CRI 1, and column

$$[ \begin{matrix}{e}^{- j 2 π {f}_{0} {t}_{N - 1}} \\ {e}^{- j 2 π {f}_{1} {t}_{N - 1}} \\ ⋮ \\ {e}^{- j 2 π {f}_{N - 1} {t}_{N - 1}}\end{matrix} ]$$

represents the value of TD-CRI N−1.

In an implementation, sensing agent 516 may use the TD bit map to remove columns of A_{F,N} which correspond to zeros in the TD bit map. For a bit weight of k, the matrix is now A_{F,k} (given by equation (27)).

$${A}_{F , k} = [ \begin{matrix}{e}^{- j 2 π {f}_{0} {t}_{0}} & {e}^{- j 2 π {f}_{0} {t}_{1}} & … & {e}^{- j 2 π {f}_{0} {t}_{k - 1}} \\ {e}^{- j 2 π {f}_{1} {t}_{0}} & {e}^{- j 2 π {f}_{1} {t}_{1}} & … & {e}^{- j 2 π {f}_{1} {t}_{k - 1}} \\ ⋮ & & & ⋮ \\ {e}^{- j 2 π {f}_{N - 1} {t}_{0}} & … & & {e}^{- j 2 π {f}_{N - 1} {t}_{k - 1}}\end{matrix} ] \qquad ( 27 )$$

In an example, the full TD-CRI includes the same channel representation information as the CSI, however the information may be concentrated in only a few of the time domain pulses. In an example, CRI may be represented with less data by only sending the time domain pulses that are needed. In an implementation, optimum time domain pulses required to represent the CSI at a defined level of accuracy may be determined. The optimum number of time domain pulses required to represent the CSI at a defined level of accuracy may be referred to as principal impulses. According to an implementation, the level of accuracy may be defined by setting out a maximum error that is allowable between the CSI and the R-CSI.

According to an implementation, sensing agent 516 may be configured to identify principal impulses of the full TD-CRI. In an example, the principal impulses may represent a subset of time domain pulses of the full TD-CRI. The subset of time domain pulses may include the optimum time domain pulses required to represent the CSI accurately. In an implementation, sensing agent 516 may identify a filtered TD-CRI according to the principal impulses. The filtered TD-CRI may be an example of channel representation information.

In an implementation, sensing agent 516 may identify the principal impulses of the full TD-CRI based on constraint processing. An example of the constraint processing is described hereinafter.

In an implementation, sensing agent 516 may identify a subset of time domain complex pairs of the full TD-CRI. Sensing agent 516 may then use the subset of time domain complex pairs as an initial filtered TD-CRI representation of the propagation channel. According to an implementation, sensing agent 516 may filter the full TD-CRI using Fourier matrix representations. An expansion of the matrix A_{F,k} is shown in equation (28) below.

$${A}_{F , k} = [ \begin{matrix}e ( 0 , 0 ) & e ( 0 , 1 ) & … & e ( 0 , 6 ) & e ( 0 , 7 ) & e ( 0 , 8 ) & e ( 0 , 9 ) & … & e ( 0 , k - 1 ) \\ e ( 1 , 0 ) & e ( 1 , 1 ) & … & e ( 1 , 6 ) & e ( 1 , 7 ) & e ( 1 , 8 ) & e ( 1 , 9 ) & … & e ( 1 , k - 1 ) \\ … & … & & e ( 2 , 6 ) & e ( 2 , 7 ) & e ( 2 , 8 ) & e ( 2 , 9 ) & … & … \\ & & & … & … & … & … & & \\ e ( N - 1 , 0 ) & e ( N - 1 , 1 ) & & e ( N - 1 , 6 ) & e ( N - 1 , 7 ) & e ( N - 1 , 8 ) & e ( N - 1 , 9 ) & & e ( N - 1 , k - 1 )\end{matrix} ] \qquad ( 28 )$$

For simplicity of notation, in equation (28), e^{-j2πf}^{₀}^{t}^{₀} is written as e(0,0), e^{-j2πf}^{₁}^{t}^{₀} is written as e(1,0), and so on.

In an implementation, sensing agent 516 may constrain the matrix A_{F,k} by eliminating the columns that do not contribute to the channel representation by some measure keeping the columns that do contribute to the channel representation by the same measure (i.e., principal impulses) In an example, it may be assumed that the set of contributing αᵢ occurs for i={6, 7, 8, 9} and is referred to as c as shown in equation (29).

$$α = [ \begin{matrix}{α}_{6} \\ {α}_{7} \\ {α}_{8} \\ {α}_{9}\end{matrix} ] \qquad ( 29 )$$

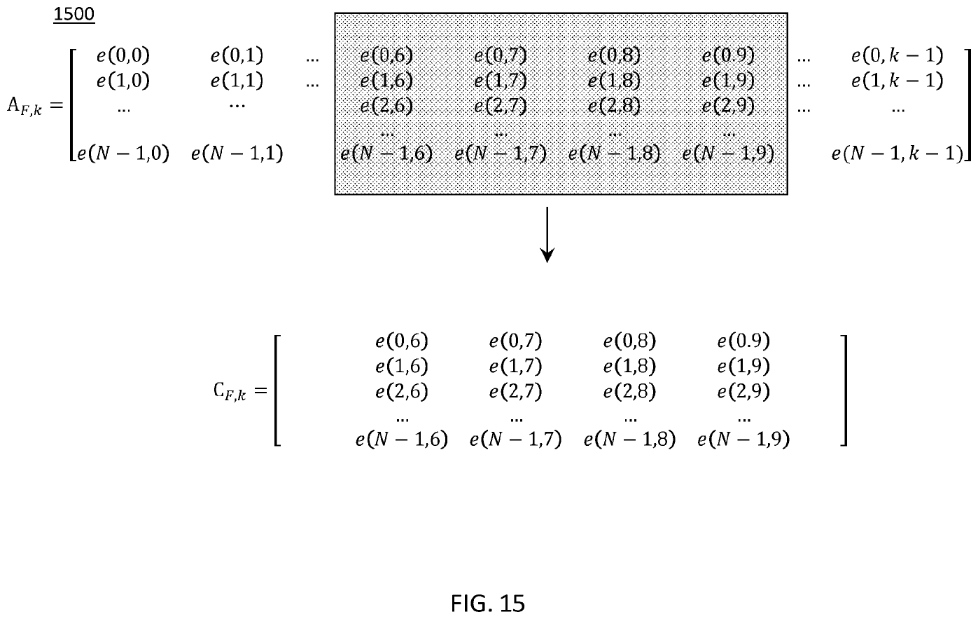

The constrained version of A_{F,k} (also referred to as constrained basis matrix C_{F,k}) is created by only keeping the set of column numbers that correspond to the contributing at, that is columns {6, 7, 8, 9}. Example 1500 of the creation of the constrained basis matrix C_{F,k} is depicted in FIG. 15. As depicted in FIG. 15, the constrained basis matrix C_{F,k} is created by keeping the set of column numbers that correspond to the contributing αᵢ.

In an implementation, sensing agent 516 may update the TD bit map with zeros (0s) for additional time domain pulses that are removed. The new bit weight of the TD bit map is calculated to be m. Sensing agent 516 may update the column vector resulting in a new (smaller) column vector c of length m. In an example implementation, the column vector c is represented using equation (30), provided below.

$$α = [ \begin{matrix}{α}_{0} \\ {α}_{1} \\ … \\ {α}_{m - 1}\end{matrix} ] \qquad ( 30 )$$

In an implementation, using the updated TD bit map, sensing agent 516 may remove the columns of the matrix A_{F,k} which correspond to the new zeros in the TD bit map. For a bit weight (number of 1's in the TD bit map) of m, the matrix A_{F,k} is now referred to as C_{F,m}. The m columns represent the principal impulses. The matrix C_{F,m} may be represented using equation (31), provided below.

$${C}_{F , m} = [ \begin{matrix}{e}^{- j 2 π {f}_{0} {t}_{0}} & {e}^{- j 2 π {f}_{0} {t}_{1}} & … & {e}^{- j 2 π {f}_{0} {t}_{m - 1}} \\ {e}^{- j 2 π {f}_{1} {t}_{0}} & {e}^{- j 2 π {f}_{1} {t}_{1}} & … & {e}^{- j 2 π {f}_{1} {t}_{m - 1}} \\ ⋮ & & & ⋮ \\ {e}^{- j 2 π {f}_{N - 1} {t}_{0}} & … & & {e}^{- j 2 π {f}_{N - 1} {t}_{m - 1}}\end{matrix} ] \qquad ( 31 )$$

In an implementation, although sensing receiver 502 may calculate the CSI of the entire wideband signal, only the CSI associated with the component frequency bands of each sensing transmitter is relevant for Wi-Fi sensing calculations over the channel between that sensing transmitter and sensing receiver 502. According to an implementation, sensing agent 516 may identify component frequency bands associated with a virtual wideband sensing transmission from a selected sensing transmitter of plurality of sensing transmitters 504-(1-M). In an example, the selected sensing transmitter may be sensing transmitter 504-1. According to an example, in an 80 MHz channel bandwidth, sensing transmitter 504-1 may have three component frequency bands which together form a virtual wideband. In an implementation, sensing receiver 502 may receive all the component frequency bands in the virtual wideband at the same time (i.e., in the same TXOP).

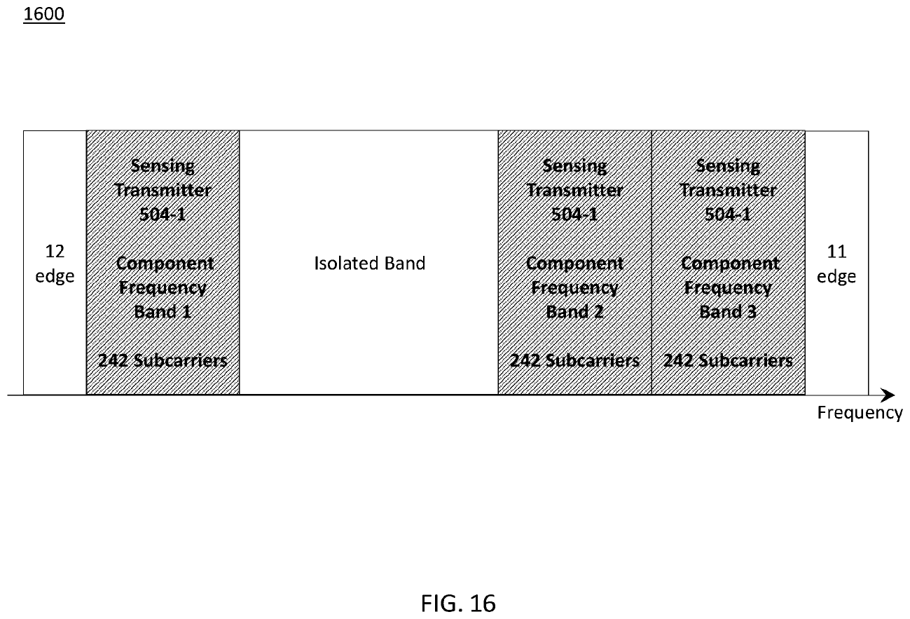

FIG. 16 depicts example 1600 of a virtual wideband of sensing transmitter 504-1 in an 80 MHz channel bandwidth, according to some embodiments. As depicted in FIG. 16, three component frequency bands, namely, component frequency band 1, component frequency band 2, and component frequency band 3 are assigned to sensing transmitter 504-1. In an example, each of the component frequency band 1, component frequency band 2, and component frequency band 3 has 242 subcarriers. The subcarriers marked as “edge” in FIG. 16 are null subcarriers. Although it has been shown that each component frequency band (i.e., each of the component frequency band 1, component frequency band 2, and component frequency band 3) has 242 subcarriers, each component frequency band may be of any size and may be different sizes to each other.

According to an example, a number of subcarriers of all the component frequency bands assigned to sensing transmitter 504-1 may be M_{c|A}. Accordingly, the virtual wideband for sensing transmitter 504-1 may be M_{c|a} subcarriers wide. In an example, a number of subcarriers of an isolated band may be M_{i|a}. According to an example, upon receiving an 80 MHz OFDMA signal, sensing receiver 502 may calculate CSI on the entire 80 MHz received signal. The total number of subcarriers of all component frequency bands and the isolated band is M_{c|a}+M_{i|A}. However, of the M_{c|A}+M_{i|a} subcarriers, only the CSI calculated on M_{c|a} subcarriers is relevant for Wi-Fi sensing calculations for sensing transmitter 504-1. In an implementation, the isolated band may be used for a sensing transmission from a different sensing transmitter (for example, sensing transmitter 504-2).

FIG. 17 depicts example 1700 of an UL-OFDMA signal received by sensing receiver 502, according to some embodiments. In an example implementation, the UL-OFDMA signal may include sensing transmissions from two sensing transmitters, i.e., from sensing transmitter 504-1 and sensing transmitter 504-2. In the example implementation, multiple component frequency bands assigned to sensing transmitter 504-1 form a virtual wideband for sensing transmitter 504-1. As depicted in FIG. 17, three component frequency bands, namely, component frequency band 1, component frequency band 2, and component frequency band 3 are assigned to sensing transmitter 504-1. In an example, each of the component frequency band 1, component frequency band 2, and component frequency band 3 has 242 subcarriers. Further, a single component frequency band, component frequency band 4, is assigned to sensing transmitter 504-2. Component frequency band 4 assigned to sensing transmitter 504-2 has 242 subcarriers. FIG. 17 also shows 7 subcarriers in the center which are DC tones as well as a further, 13 subcarriers on either side of the 7 subcarriers, 12 edge subcarriers, and 11 edge subcarriers which are unused for the uplink sensing transmission received by sensing receiver 502.

Referring back to FIG. 5, in an implementation, sensing receiver 502 may process the CSI from all component frequency bands assigned to same sensing transmitter as a whole entity. According to an implementation, sensing agent 516 may be configured to identify component frequency bands associated with a virtual wideband sensing transmission from a selected sensing transmitter of plurality of sensing transmitters 504-(1-M). In an example, the selected sensing transmitter may be sensing transmitter 504-1 and remainder of the plurality of sensing transmitters may include sensing transmitters 504-(2-M). In some examples, the selected sensing transmitter may be any sensing transmitter from amongst plurality of sensing transmitters 504-(1-M). In an example, the component frequency bands associated with the sensing transmission may be contiguous bands within a transmission channel. In some examples, the component frequency bands associated with the sensing transmission may include non-contiguous bands within the transmission channel.

Upon identifying the component frequency bands associated with the virtual wideband sensing transmission from the selected sensing transmitter of plurality of sensing transmitters 504-(1-M), sensing agent 516 may generate a reduced filtered TD-CRI including the component frequency bands associated with the selected sensing transmitter and omitting component frequency bands associated with a remainder of plurality of sensing transmitters.

According to an implementation, sensing agent 516 may be configured to create a frequency domain (FD) bit map. The FD bit map may indicate locations of frequencies in the Fourier basis matrix that align with the component frequency bands associated with the virtual wideband sensing transmission. In an example, the FD bit map may be 1024 bits long. In an implementation, sensing agent 516 may populate the FD bit map with ones (1s) at the location of component frequency band subcarriers and zeros (0s) at the location of edge subcarriers, subcarriers used by other sensing transmitters, DC tones, and unused subcarriers. In an example, the FD bit map may be populated according to table 9, provided below:

### Table 9: FD Bit Map Positions and Value

TABLE 9
FD Bit map positions and value

| FD Bit map positions | Value |
|---|---|
| 0-11 | 0 |
| 12-253 | 1 |
| 254-528 | 0 |
| 529-1012 | 1 |
| 1013-1023 | 0 |

According to an implementation, sensing agent 516 may remove the rows of C_{F,m} given by equation (31) corresponding to the bit positions in the FD bit map that are zero. The number of remaining rows may be equivalent to the bit weight of the FD bit map. The bit weight of the FD bit map may be p. In an implementation, sensing agent 516 may concatenate the remaining rows in a contiguous manner such that the remaining matrix is of dimension p×m, and is referred as F_{p,m} (given by equation (32), provided below). The matrix F_{p,m} may include principal impulses for the selected sensing transmitter.

$${F}_{p , m} = [ \begin{matrix}{e}^{- j 2 π {f}_{0} {t}_{0}} & {e}^{- j 2 π {f}_{0} {t}_{1}} & … & {e}^{- j 2 π {f}_{0} {t}_{m - 1}} \\ {e}^{- j 2 π {f}_{1} {t}_{0}} & {e}^{- j 2 π {f}_{1} {t}_{1}} & … & {e}^{- j 2 π {f}_{1} {t}_{m - 1}} \\ ⋮ & & & ⋮ \\ {e}^{- j 2 π {f}_{p - 1} {t}_{0}} & … & & {e}^{- j 2 π {f}_{p - 1} {t}_{m - 1}}\end{matrix} ] \qquad ( 32 )$$

According to an implementation, sensing agent 516 may store the principal impulses of the full TD-CRI and/or the principal impulses of the reduced filtered TD-CRI (i.e., the principal impulses of the selected sensing transmitter) in channel representation information storage 518 for future use.

In an implementation, sensing agent 516 may send the reduced filtered TD-CRI to sensing algorithm manager 506. Sensing agent 516 may also send location information indicating locations of the principal impulses in the full TD-CRI to sensing algorithm manager 506. In an example, the location information may be included in the reduced filtered TD-CRI. In an implementation, sensing agent 516 may communicate the reduced filtered TD-CRI and corresponding location information to sensing algorithm manager 506 via a CRI transmission message. In an example implementation, sensing agent 516 may communicate the CRI transmission message including the reduced filtered TD-CRI and corresponding location information to sensing algorithm manager 506. In an implementation, sensing agent 516 may encode the reduced filtered TD-CRI and corresponding location information for transmission to sensing algorithm manager 506 over the air via transmitting antenna 512.

In an example, the location information may represent positions of the principal impulses in a Fourier basis matrix. In an example, the location information may include one or more bit maps. In an implementation, sensing algorithm manager 506 may be required to create a reconstructed TD-CRI from the reduced filtered TD-CRI prior to performing a DFT to create a R-CSI. In an implementation, for sensing algorithm manager 506 to correctly create the reconstructed TD-CRI, the sensing algorithm manager 506 may identify where to place each of the filtered TD-CRI complex values that it receives from sensing receiver 502 in the reconstructed TD-CRI prior to performing the DFT.

When the reduced filtered TD-CRI is generated, the selection of the time domain pulses that are kept (i.e., the principal impulses) is captured in the indices of the values of a that are captured in c. Therefore, for sensing algorithm manager 506 to determine how to create the reconstructed TD-CRI from the values that sensing algorithm manager 506 receives over the air from sensing receiver 502, sensing algorithm manager 506 is required to identify in which columns and in which rows of the Fourier basis matrix, the received reduced filtered TD-CRI should be located.

FIG. 18 illustrates representation 1800 of communication of locations of the principal impulses in a Fourier basis matrix from sensing receiver 502 to sensing algorithm manager 506 using a TD bit map and an FD bit map, according to some embodiments. In an example, the TD bit map describes active tones. In an example, information of locations of the principal impulses is included in the reduced filtered TD-CRI. According to an example of FIG. 18, the TD bit map sent from sensing receiver 502 to sensing algorithm manager 506 is 10 bits long, corresponding to 10 pilot and data tones of a 16-point DFT. The value of the TD bit map, “1110111011” indicates that 8 filtered TD-CRI values will follow (as there are 8 “1″”s in the active tone bitmap) and sensing algorithm manager 506 should arrange the received reduced filtered TD-CRI by applying, in order, each reduced filtered TD-CRI to a reconstructed TD-CRI tone according to the active tone bit map, i.e., TD-CRI 1 in tone 1, TD-CRI 2 in tone 2, TD-CRI 3 in tone 3, null in tone 4, TD-CRI 4 in tone 5, TD-CRI 5 in tone 6, TD-CRI 6 in tone 7, null in tone 8, TD-CRI 7 in tone 9, and TD-CRI 8 in tone 10. Furthermore in the example of FIG. 18 the FD bit map sent from sensing receiver 502 to sensing algorithm manager 506 is 10 bits long, corresponding to 10 frequency points in the Fourier basis matrix. The value of the FD bit map, “1110000111” indicates that the Fourier basis matrix describes two bands in the virtual wideband and a location of an isolated band. In an embodiment, sensing agent 516 of sensing receiver 502 may communicate the locations of the principal impulses.

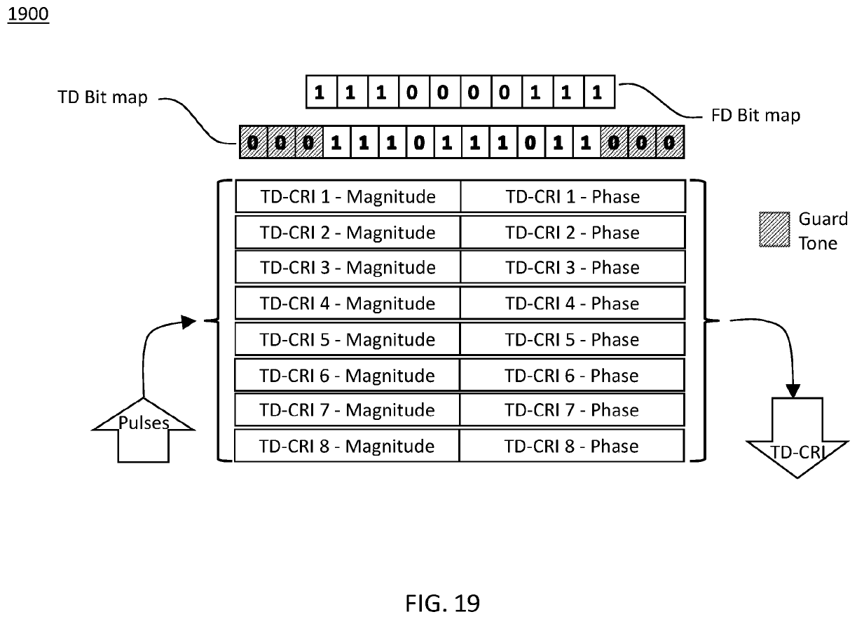

FIG. 19 illustrates representation 1900 of communication of locations of the principal impulses in a Fourier basis matrix from sensing receiver 502 to sensing algorithm manager 506 using a TD bit map and an FD bit map, according to some embodiments. In an example, the TD bit map describes full tones. In an example, information of locations of the principal impulses is included in the reduced filtered TD-CRI. According to an example of FIG. 19, the TD bit map may be equal to the total number of tones in the full TD-CRI including the guard tones and DC tones, e.g., 64 bits for an example of a 20 MHz propagation channel bandwidth and 128 bits for an example of 40 MHz propagation channel bandwidth. In the example, some most significant bits (MSB) are “0” to account for the guard tones and some least significant bits (LSB) are “0” to account for the DC tone and the guard tones. In the 16-point DFT example shown in FIG. 19, zeros are placed in the first three locations of the full bit map followed by the location of the eight TD-CRI, followed by three more zeros. Furthermore in the example of FIG. 19 the FD bit map sent from sensing receiver 502 to sensing algorithm manager 506 is 10 bits long, corresponding to 10 frequency points in the Fourier basis matrix. The value of the FD bit map, “1110000111” indicates that the Fourier basis matrix describes two bands in the virtual wideband and a location of an isolated band. In an embodiment, sensing agent 516 of sensing receiver 502 may communicate the locations of the principal impulses.

According to some implementations, for each reduced filtered TD-CRI, communicated from sensing receiver 502 to sensing algorithm manager 506, sensing receiver 502 may send three values instead of two values (first value being amplitude of the complex number and second value being phase of the complex number) and an FD bit map. In an example, the third value may represent the locations of the principal impulses in the full TD-CRI. In an example, the number of bits used to represent the third value may vary depending on the channel bandwidth and therefore the number of pulses in the full TD-CRI. For example, if the channel bandwidth is 20 MHz, a 64-point DFT is required and thus the additional value may be 6 bits long. If the channel bandwidth is 40 MHz, a 128-point DFT is required and thus the additional value may be 7 bits long. In an example, the additional value could precede the values of reduced filtered TD-CRI. In some examples, the additional value could follow the values of reduced filtered TD-CRI. In an example, the number of bits used for the reduced filtered TD-CRI may be determined based on the resolution of the actual CSI output by the baseband receiver.

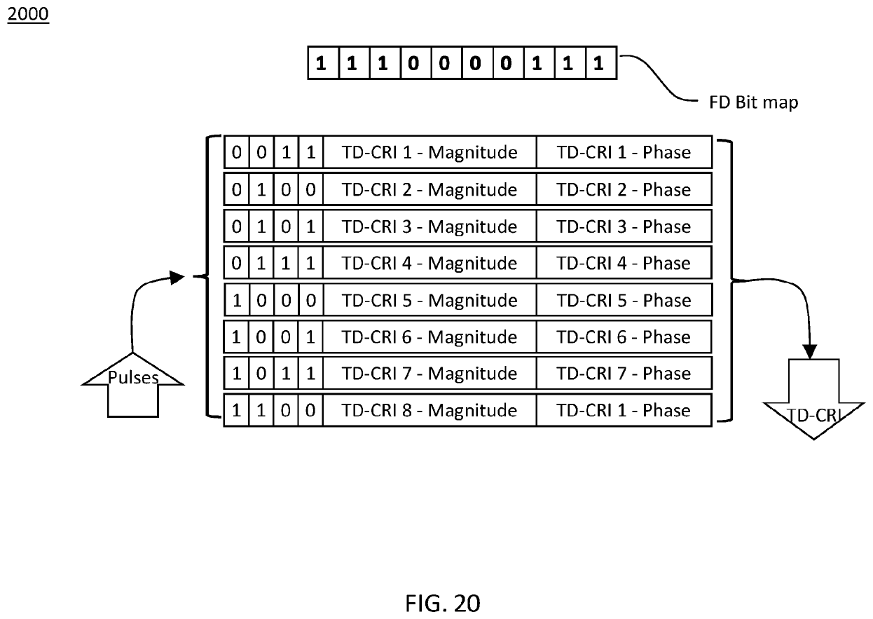

FIG. 20 illustrates representation 2000 of communication of locations of the principal impulses from sensing receiver 502 to sensing algorithm manager 506 using positions of the principal impulses in the full TD-CRI, according to some embodiments. In an example of FIG. 20, the numbering of the symbols has been shifted to start at “0” and end at “15” to facilitate mapping of the symbols to the position of the reduced filtered TD-CRI value in the reconstructed TD-CRI. Furthermore in the example of FIG. 20 the FD bit map sent from sensing receiver 502 to sensing algorithm manager 506 is 10 bits long, corresponding to 10 frequency points in the Fourier basis matrix. The value of the FD bit map, “1110000111” indicates that the Fourier basis matrix describes two bands in the virtual wideband and a location of an isolated band. In an embodiment, sensing agent 516 of sensing receiver 502 may communicate the locations of the principal impulses.

FIG. 18 to FIG. 20 illustrate examples of communication of the principal impulses in the full TD-CRI utilizing 16-point DFT with 3 guard tones on either side (leaving 10 tones for pilot and data symbols), however the description is equally applicable to 32-point DFT, 64-point DFT, 128-point DFT, 256-point DFT, 512-point DFT, 1024-point DFT, and any other number of points in an DFT, and a variable number of guard tones.

In an example implementation, sensing agent 516 may send the TD bit map and the FD bit map to sensing algorithm manager 506. In an example, the TD bit map may indicate the columns (for example, left to right) and the FD bit map may indicate the rows (for example, top to bottom) in which to locate the reduced filtered TD-CRI (in the order that they are received) to calculate the R-CSI. In another examples, each reduced filtered TD-CRI value may include three parts: a value which represents the column (or DFT tone number) that the reduced filtered TD-CRI value belongs in, and an amplitude and a time delay value of the principal impulse. Accordingly, for each reduced filtered TD-CRI, sensing agent 516 may send the FD bit map indicating the row of each of the communicated reduced filtered TD-CRI and three values-{k, αₖ, and tₖ}.

In response to receiving the reduced filtered TD-CRI and corresponding location information, sensing agent 536 may be configured to generate a reconstructed TD-CRI prior to performing a DFT to create an R-CSI. In an implementation, sensing agent 536 may generate the reconstructed TD-CRI from the reduced filtered TD-CRI and the location information. According to an example, sensing agent 536 may use the location information to determine placement of the principal impulses in the reconstructed TD-CRI. Sensing agent 536 may then transform the reconstructed TD-CRI into R-CSI.

According to an implementation, sensing agent 536 may construct an empty Fourier basis matrix of dimension N×m(D_{N,m}). In an implementation, according to the FD bit map, for each bit position in the FD bit map, where there is a zero (0), sensing agent 536 may populate the corresponding row of D_{N,m} with all zeros (0s). In an implementation, sensing agent 536 may fill in the p non-zero rows of D_{N,m} with the values of F_{p,m}. In an example sensing agent 536 may process the rows and then the columns of D_{N,m} filing in values of F_{p,m}. An example code excerpt for filling in the p non-zero rows of D_{N,m} with the values of F_{p,m} is provided below.

| | |
|---|---|
| s = 0 | // s is a column index |
| q = 0 | // q is a row index of D_{N,m} |
| r = 0 | // r is a row index of F_{p,m} |
| For q = 0 to (N − 1) | // for each of the rows of D_{N,m} |
| If D_{q,s} ≠ 0 | // if this cell of the matrix has not already |

been set to zero

| | |
|---|---|
| For s = 0 to (m − 1) | // for each of the columns in |

that row

| | |
|---|---|
| D_{q,s} = F_{r,s} | // place a value of F_{p,m} |

```
    End For
    r = r + 1 (move to the next row of F_{p,m})
  End if
```

| | |
|---|---|
| s = 0 | // start again at the first column of D_{N,m} |

End For

According to an implementation, sensing agent 536 may calculate the R-CSI for the selected transmitter using equation (33), provided below.

$${D}_{N , m} \times c = {H}_{R} ( f ) \qquad ( 33 )$$

In an implementation, sensing agent 536 may use the FD bit map to remove the rows of the R-CSI that are not part of the component frequency bands assigned to the selected sensing transmitter. The resulting R-CSI is then used for Wi-Fi sensing for the selected sensing transmitter. In some implementations, the rows of the reduced filtered TD-CRI are arranged contiguous in a reduced dimension reconstructed Fourier basis matrix and a reduced dimension DFT is performed to calculate the R-CSI which is then used for Wi-Fi sensing for the selected sensing transmitter. In an implementation, sensing agent 536 may execute a sensing algorithm on the R-CSI to obtain a sensing result, such as detecting motions or gestures.

Although, it has been described that sensing receiver 502 first generates the filtered TD-CRI and then reduces the filtered TD-CRI to the parts of the virtual wideband sensing transmission from the selected sensing transmitter, thereby generating the reduced filtered TD-CRI, in some embodiments, it may not be required to generate the filtered TD-CRI, and instead sensing receiver 502 may generate a reduced CRI. In an example, the reduced CRI may be a reduced TD-CRI. The reduced TD-CRI may include time domain representations of the component frequency bands associated with the selected sensing transmitter and omitting component frequency bands associated with the remainder of the plurality of sensing transmitters. The reduced TD-CRI may further include FD bit map indicating locations of the time domain representations in the full TD-CRI. In an implementation, sensing receiver 502 may send the reduced CRI to sensing algorithm manager 506 for obtaining a sensing result.

When the encoded reduced filtered TD-CRI is received by sensing algorithm manager 506, a reconstruction of the CSI is made from the information of the reduced filtered TD-CRI. In an example, the correctly positioned reconstructed TD-CRI, when translated back to the frequency domain via the DFT, creates the R-CSI. In an implementation, since there are significantly fewer reduced filtered TD-CRI than CSI values then there is a significant reduction in the amount of information that needs to be transmitted over the air as CRI to sensing algorithm manager 506 without losing the fidelity of the information which would compromise the performance of sensing algorithm manager 506. Accordingly, minimizing the amount of information that needs to be sent minimizes the overhead that system 500 puts on network 560. Further, the CRI that is sent to sensing algorithm manager 506 enables the R-CSI for a virtual wideband sensing transmission from the selected sensing transmitter to appear as though the selected sensing transmitter actually transmitted a wideband signal, such that the R-CSI may be used to determine movement or motion.

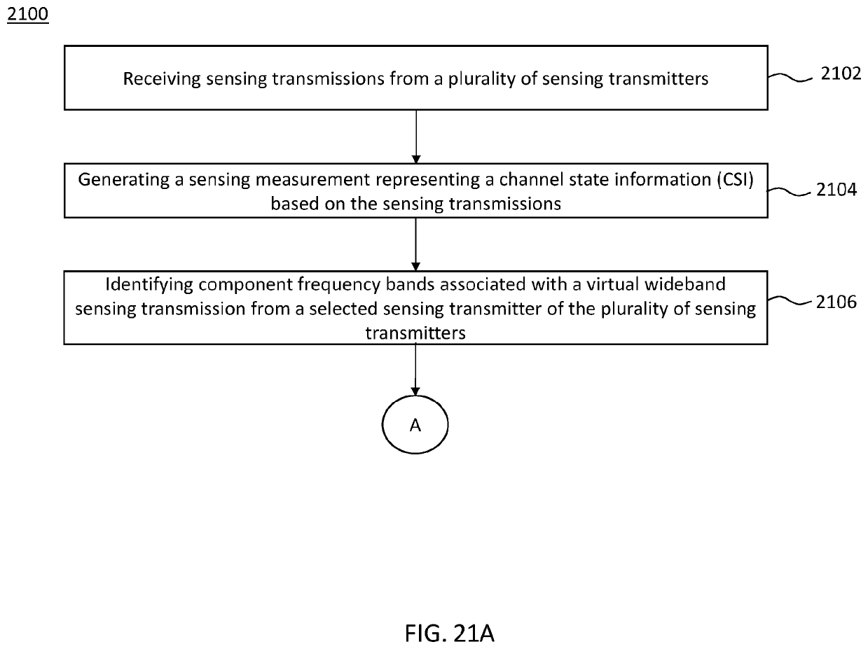

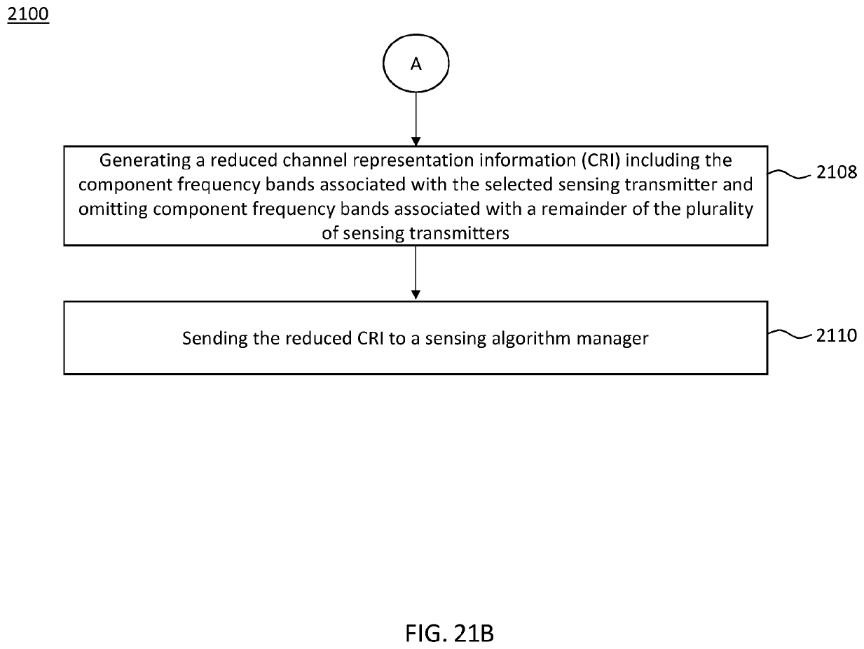

FIG. 21A and FIG. 21B depict flowchart 2100 for sending a reduced CRI to sensing algorithm manager 506, according to some embodiments.

In a brief overview of an implementation of flowchart 2100, at step 2102, sensing transmissions from plurality of sensing transmitters 504-(1-M) are received. At step 2104, a sensing measurement representing CSI is generated based on the sensing transmissions. At step 2106, component frequency bands associated with a virtual wideband sensing transmission from a selected sensing transmitter of plurality of sensing transmitters 504-(1-M) are identified. At step 2108, a reduced CRI including the component frequency bands associated with the selected sensing transmitter and omitting component frequency bands associated with a remainder of the plurality of sensing transmitters is generated. At step 2110, the reduced CRI is sent to sensing algorithm manager.

Step 2102 includes receiving sensing transmissions from a plurality of sensing transmitters. In an implementation, sensing receiver 502 may be configured to receive the sensing transmissions from plurality of sensing transmitters 504-(1-M).

Step 2104 includes generating a sensing measurement representing CSI based on the sensing transmissions. In an implementation, sensing receiver 502 may be configured to generate the sensing measurement representing CSI based on the sensing transmissions.

Step 2106 includes identifying component frequency bands associated with a virtual wideband sensing transmission from a selected sensing transmitter of the plurality of sensing transmitters. In an implementation, sensing receiver 502 may be configured to identify the component frequency bands associated with the virtual wideband sensing transmission from the selected sensing transmitter of plurality of sensing transmitters 504-(1-M). In an example, the component frequency bands associated with the sensing transmission are contiguous bands within a transmission channel. In some examples, the component frequency bands associated with the sensing transmission include non-contiguous bands within the transmission channel.

Step 2108 includes generating a reduced CRI including the component frequency bands associated with the selected sensing transmitter and omitting component frequency bands associated with a remainder of plurality of sensing transmitters or that are not allocated. In an implementation, sensing receiver 502 may be configured to generate the reduced CRI including the component frequency bands associated with the selected sensing transmitter and omitting component frequency bands associated with a remainder of plurality of sensing transmitters or that are not allocated.

In an implementation, generating the reduced CRI includes generating a full TD-CRI of the CSI, generating, a reduced TD-CRI including time domain representations of the component frequency bands associated with the selected sensing transmitter and omitting component frequency bands associated with the remainder of the plurality of sensing transmitters, and generating a FD bit map indicating locations of the time domain representations in the full TD-CRI.

In some implementations, generating the reduced CRI further includes generating a reduced filtered TD-CRI including principal impulses of the reduced TD-CRI, the principal impulses representing a subset of time domain pulses of the full TD-CRI, and generating location information indicating locations of the principal impulses in the reduced TD-CRI. In an example, the principal impulses are selected to permit reconstruction of the reduced TD-CRI. In another example, the location information includes a bit map and in a further example, the location information is included in the reduced filtered TD-CRI.

Step 2110 includes sending the reduced CRI to sensing algorithm manager 506. In an implementation, sensing receiver 502 may be configured to send the reduced CRI to sensing algorithm manager 506.

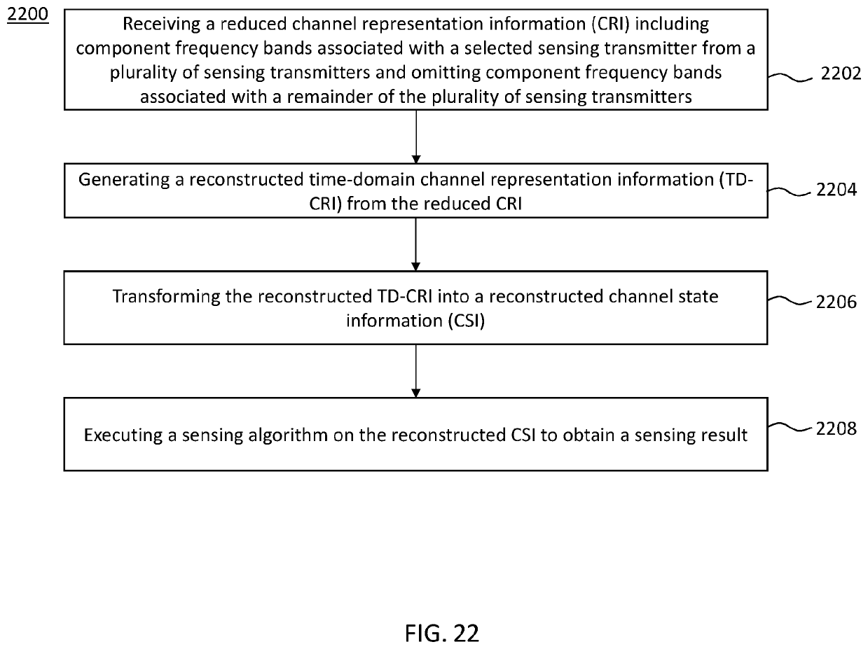

FIG. 22 depicts flowchart 2200 for executing a sensing algorithm on a reconstructed CSI to obtain a sensing result, according to some embodiments.

In a brief overview of an implementation of flowchart 2200, at step 2202, a reduced CRI, including component frequency bands associated with a selected sensing transmitter from a plurality of sensing transmitters and omitting component frequency bands associated with a remainder of the plurality of sensing transmitters, is received. At step 2204, a reconstructed TD-CRI is generated from the reduced CRI. At step 2206, the reconstructed TD-CRI is transformed into a reconstructed CSI. At step 2208, a sensing algorithm is executed on the reconstructed CSI to obtain a sensing result.

Step 2202 includes receiving a reduced CRI including component frequency bands associated with a selected sensing transmitter from a plurality of sensing transmitters and omitting component frequency bands associated with a remainder of the plurality of sensing transmitters. According to an implementation, sensing algorithm manager 506 may receive the reduced CRI including the component frequency bands associated with the selected sensing transmitter from plurality of sensing transmitters 504-(1-M) and omitting the component frequency bands associated with the remainder of the plurality of sensing transmitters.

In an implementation, the reduced CRI is a reduced TD-CRI including time domain representations of the component frequency bands associated with the selected sensing transmitter and omitting component frequency bands associated with a remainder of the plurality of sensing transmitters, and a FD bit map indicating locations of the time domain representations in a full TD-CRI.

In some implementations, the reduced CRI is a reduced filtered TD-CRI including time domain representations of principal impulses of the component frequency bands associated with the selected sensing transmitter and omitting component frequency bands associated with a remainder of the plurality of sensing transmitters, a FD bit map indicating the locations of the time domain representations in a full TD-CRI, and location information indicating the locations of the principal impulses in the full TD-CRI.

Step 2204 includes generating a reconstructed TD-CRI from the reduced CRI. According to an implementation, sensing algorithm manager 506 may generate the reconstructed TD-CRI from the reduced CRI. In an implementation, sensing algorithm manager 506 may generate the reconstructed TD-CRI based on the location information, the FD bit map, and the principal impulses of the reduced filtered TD-CRI.

Step 2206 includes transforming the reconstructed TD-CRI into a reconstructed CSI. According to an implementation, sensing algorithm manager 506 may transform the reconstructed TD-CRI into the reconstructed CSI.

Step 2208 includes executing a sensing algorithm on the reconstructed CSI to obtain a sensing result. According to an implementation, sensing algorithm manager 506 may execute the sensing algorithm on the reconstructed CSI to obtain the sensing result.

While various embodiments of the methods and systems have been described, these embodiments are illustrative and in no way limit the scope of the described methods or systems. Those having skill in the relevant art can effect changes to form and details of the described methods and systems without departing from the broadest scope of the described methods and systems. Thus, the scope of the methods and systems described herein should not be limited by any of the illustrative embodiments and should be defined in accordance with the accompanying claims and their equivalents.

Additional embodiments consistent with the disclosure include at least the following.

Embodiment 1 is a method for Wi-Fi sensing carried out by a sensing receiver including a transmitting antenna, a receiving antenna, and at least one processor configured to execute instructions, the method comprising: receiving, via the receiving antenna, sensing transmissions from a plurality of sensing transmitters; generating, by the at least one processor, a sensing measurement representing a channel state information (CSI) based on the sensing transmissions; identifying, by the at least one processor, component frequency bands associated with a virtual wideband sensing transmission from a selected sensing transmitter of the plurality of sensing transmitters; generating, by the at least one processor, a reduced channel representation information (CRI) including the component frequency bands associated with the selected sensing transmitter and omitting component frequency bands associated with a remainder of the plurality of sensing transmitters; and sending the reduced CRI to a sensing algorithm manager.

Embodiment 2 is the method of embodiment 1, wherein the component frequency bands associated with the sensing transmission are contiguous bands within a transmission channel.

Embodiment 3 is the method of embodiment 1 or 2, wherein the component frequency bands associated with the sensing transmission include non-contiguous bands within a transmission channel.

Embodiment 4 is the method of any of embodiments 1-3, wherein generating the reduced CRI includes: generating, by the at least one processor, a full time-domain channel representation information (TD-CRI) of the CSI; generating, by the at least one processor, a reduced TD-CRI including time domain representations of the component frequency bands associated with the selected sensing transmitter and omitting component frequency bands associated with a remainder of the plurality of sensing transmitters; and generating, by the at least one processor, a frequency domain bit map indicating the locations of the time domain representations in the full TD-CRI.

Embodiment 5 is the method of embodiment 4, further comprising: generating a reduced filtered TD-CRI including principal impulses of the reduced TD-CRI, the principal impulses representing a subset of time domain pulses of the full TD-CRI; and generating location information indicating locations of the principal impulses in the reduced TD-CRI.

Embodiment 6 is the method of embodiment 5, wherein the principal impulses are selected to permit reconstruction of the reduced TD-CRI.

Embodiment 7 is the method of embodiment 5 or 6, wherein the location information includes a bit map.

Embodiment 8 is the method of any of embodiments 5-7, wherein the location information is included in the reduced filtered TD-CRI.

Embodiment 9 is the method of any of embodiments 1-8, further comprising: obtaining, by the sensing algorithm manager, the reduced CRI; generating, by the sensing algorithm manager, a reconstructed CSI based on the reduced CRI; and executing, by the sensing algorithm manager, a sensing algorithm according to the reconstructed CSI to obtain a sensing result.

Embodiment 10 is the method of any of embodiments 5-9, further comprising: obtaining, by the sensing algorithm manager, the reduced filtered TD-CRI, the location information, and the frequency domain bit map; generating, by the sensing algorithm manager, a reconstructed TD-CRI based on the location information, the frequency domain bit map, and the principal impulses of the filtered TD-CRI; generating, by the sensing algorithm manager, a reconstructed CSI according to the reconstructed TD-CRI; and executing, by the sensing algorithm manager, a sensing algorithm according to the reconstructed CSI to obtain a sensing result.

Embodiment 11 is a method for Wi-Fi sensing carried out by a device including a receiving antenna and at least one processor configured to execute instructions, the method comprising: receiving, via the receiving antenna, a reduced channel representation information (CRI) including component frequency bands associated with a selected sensing transmitter from a plurality of sensing transmitters and omitting component frequency bands associated with a remainder of the plurality of sensing transmitters; generating, by a sensing algorithm manager operating on the at least one processor, a reconstructed time-domain channel representation information (TD-CRI) from the reduced CRI; transforming the reconstructed TD-CRI into a reconstructed channel state information (CSI); and executing, by the sensing algorithm manager, a sensing algorithm on the reconstructed CSI to obtain a sensing result.

Embodiment 12 is the method of embodiment 11, wherein: the reduced CRI is a reduced TD-CRI including: time domain representations of the component frequency bands associated with the selected sensing transmitter and omitting component frequency bands associated with a remainder of the plurality of sensing transmitters, and a frequency domain bit map indicating the locations of the time domain representations in a full TD-CRI.

Embodiment 13 is the method of embodiment 11 or 12, wherein the reduced CRI is a reduced filtered TD-CRI including: time domain representations of principal impulses of the component frequency bands associated with the selected sensing transmitter and omitting component frequency bands associated with a remainder of the plurality of sensing transmitters, a frequency domain bit map indicating the locations of the time domain representations in a full TD-CRI, and location information indicating the locations of the principal impulses in the full TD-CRI.

Embodiment 14 is a system for Wi-Fi sensing, the system comprising: a sensing receiver including: a transmitting antenna, a receiving antenna, and at least one processor configured to execute instructions for: receiving, via the receiving antenna, sensing transmissions from a plurality of sensing transmitters; generating a sensing measurement representing a channel state information (CSI) based on the sensing transmissions; identifying component frequency bands associated with a virtual wideband sensing transmission from a selected sensing transmitter of the plurality of sensing transmitters; generating a reduced channel representation information (CRI) including the component frequency bands associated with the selected sensing transmitter and omitting component frequency bands associated with a remainder of the plurality of sensing transmitters; and sending the reduced CRI to a sensing algorithm manager.

Embodiment 15 is the system of embodiment 14, wherein the component frequency bands associated with the sensing transmission are contiguous bands within a transmission channel.

Embodiment 16 is the system of embodiment 14 or 15, wherein the component frequency bands associated with the sensing transmission include non-contiguous bands within a transmission channel.

Embodiment 17 is the system of any of embodiments 14-16, wherein generating the reduced CRI includes: generating a full time-domain channel representation information (TD-CRI) of the CSI; generating a reduced TD-CRI including time domain representations of the component frequency bands associated with the selected sensing transmitter and omitting component frequency bands associated with a remainder of the plurality of sensing transmitters; and generating a frequency domain bit map indicating the-locations of the time domain representations in the full TD-CRI.

Embodiment 18 is the system of any of embodiments 14-17, wherein the at least one processor is further configured with instructions for: generating a reduced filtered TD-CRI including principal impulses of the reduced TD-CRI, the principal impulses representing a subset of time domain pulses of the full TD-CRI; and generating location information indicating locations of the principal impulses in the reduced TD-CRI.

Embodiment 19 is the system of embodiment 18, wherein the principal impulses are selected to permit reconstruction of the reduced TD-CRI.

Embodiment 20 is the system of embodiment 18 or 19, wherein the location information includes a bit map.

Embodiment 21 is the system of any of embodiments 18-20, wherein the location information is included in the reduced filtered TD-CRI.

Embodiment 22 is the system of any of embodiments 14-21, wherein the at least one processor is further configured with instructions for: obtaining, by the sensing algorithm manager, the reduced CRI; generating, by the sensing algorithm manager, a reconstructed CSI based on the reduced CRI; and executing, by the sensing algorithm manager, a sensing algorithm according to the reconstructed CSI to obtain a sensing result.

Embodiment 23 is the system of any of embodiments 18-22, further comprising: obtaining, by the sensing algorithm manager, the reduced filtered TD-CRI, the location information, and the frequency domain bit map; generating, by the sensing algorithm manager, a reconstructed TD-CRI based on the location information, the frequency domain bit map, and the principal impulses of the filtered TD-CRI; generating, by the sensing algorithm manager, a reconstructed CSI according to the reconstructed TD-CRI; and executing, by the sensing algorithm manager, a sensing algorithm according to the reconstructed CSI to obtain a sensing result.

Embodiment 24 is a system for Wi-Fi sensing, the system comprising: a sensing receiver including: a transmitting antenna, a receiving antenna, and at least one processor configured to execute instructions for: receiving a reduced channel representation information (CRI) including component frequency bands associated with a selected sensing transmitter from a plurality of sensing transmitters and omitting component frequency bands associated with a remainder of the plurality of sensing transmitters; generating, by a sensing algorithm manager, a reconstructed time-domain channel representation information (TD-CRI) from the reduced CRI; transforming the reconstructed TD-CRI into a reconstructed channel state information (CSI); and executing, by the sensing algorithm manager, a sensing algorithm on the reconstructed CSI to obtain a sensing result.

Embodiment 25 is the system of embodiment 24, wherein: the reduced CRI is a reduced TD-CRI including: time domain representations of the component frequency bands associated with the selected sensing transmitter and omitting component frequency bands associated with a remainder of the plurality of sensing transmitters, and a frequency domain bit map indicating the locations of the time domain representations in a full TD-CRI.

Embodiment 26 is the system of embodiment 24 or 25, wherein the reduced CRI is a reduced filtered TD-CRI including: time domain representations of principal impulses of the component frequency bands associated with the selected sensing transmitter and omitting component frequency bands associated with a remainder of the plurality of sensing transmitters, a frequency domain bit map indicating the locations of the time domain representations in a full TD-CRI, and location information indicating the locations of the principal impulses in the full TD-CRI.

1. A method for Wi-Fi sensing carried out by a sensing receiver including a transmitting antenna, a receiving antenna, and at least one processor configured to execute instructions, the method comprising:
receiving, via the receiving antenna, sensing transmissions from a plurality of sensing transmitters;
generating, by the at least one processor, a one or more sensing measurements representing a channel state information (CSI) based on the sensing transmissions;
identifying, by the at least one processor, component frequency bands of a wideband channel, wherein the component frequency bands are associated with the sensing transmissions from the sensing transmitter;
generating, by the at least one processor, a reduced channel representation information (CRI) including the component frequency bands associated with the sensing transmissions from the sensing transmitter; and
sending the reduced CRI to a sensing algorithm manager.

2. The method of claim 1, wherein the component frequency bands of the wideband channel are contiguous bands within a transmission channel.

3. The method of claim 1, wherein the component frequency bands of the wideband channel include non-contiguous bands within a transmission channel.

4. The method of claim 1, wherein generating the reduced CRI includes:
generating, by the at least one processor, a full time-domain channel representation information (TD-CRI) of the CSI;
generating, by the at least one processor, a reduced TD-CRI including time domain representations of the component frequency bands of the wideband channel; and
generating, by the at least one processor, a frequency domain bit map indicating the locations of the time domain representations in the full TD-CRI.

5. The method of claim 4, further comprising:
generating a reduced filtered TD-CRI including principal impulses of the reduced TD-CRI, the principal impulses representing a subset of time domain pulses of the full TD-CRI; and
generating location information indicating locations of the principal impulses in the reduced TD-CRI.

6. The method of claim 5, wherein the principal impulses are selected to permit reconstruction of the reduced TD-CRI.

7. The method of claim 5, wherein the location information includes a bit map.

8. The method of claim 5, wherein the location information is included in the reduced filtered TD-CRI.

9. The method of claim 1, further comprising:
obtaining, by the sensing algorithm manager, the reduced CRI;
generating, by the sensing algorithm manager, a reconstructed CSI based on the reduced CRI; and
executing, by the sensing algorithm manager, a sensing algorithm according to the reconstructed CSI to obtain a sensing result.

10. The method of claim 5, further comprising:
obtaining, by the sensing algorithm manager, the reduced filtered TD-CRI, the location information, and the frequency domain bit map;
generating, by the sensing algorithm manager, a reconstructed TD-CRI based on the location information, the frequency domain bit map, and the principal impulses of the filtered TD-CRI;
generating, by the sensing algorithm manager, a reconstructed CSI according to the reconstructed TD-CRI; and
executing, by the sensing algorithm manager, a sensing algorithm according to the reconstructed CSI to obtain a sensing result.

11-13. (canceled)

14. A system for Wi-Fi sensing comprising a sensing receiver including:
a transmitting antenna, a receiving antenna, and at least one processor configured to execute instructions for:
receiving, via the receiving antenna, sensing transmissions from a plurality of sensing transmitters;
generating one or more sensing measurements representing a channel state information (CSI) based on the sensing transmissions;
identifying component frequency bands of a wideband channel, wherein the component frequency bands are associated with the sensing transmissions from the sensing transmitter;
generating a reduced channel representation information (CRI) including the component frequency bands associated with the sensing transmissions from the_sensing transmitter; and
sending the reduced CRI to a sensing algorithm manager.

15. The system of claim 14, wherein the component frequency bands of the wideband channel are contiguous bands within a transmission channel.

16. The system of claim 14, wherein the component frequency bands of the wideband channel include non-contiguous bands within a transmission channel.

17. The system of claim 14, wherein generating the reduced CRI includes:
generating a full time-domain channel representation information (TD-CRI) of the CSI;
generating a reduced TD-CRI including time domain representations of the component frequency bands of the wideband channel; and
generating a frequency domain bit map indicating the locations of the time domain representations in the full TD-CRI.

18. The system of claim 14, wherein the at least one processor is further configured with instructions for:
generating a reduced filtered TD-CRI including principal impulses of the reduced TD-CRI, the principal impulses representing a subset of time domain pulses of the full TD-CRI; and
generating location information indicating locations of the principal impulses in the reduced TD-CRI.

19. The system of claim 18, wherein the principal impulses are selected to permit reconstruction of the reduced TD-CRI.

20. The system of claim 18, wherein the location information includes a bit map.

21. The system of claim 18, wherein the location information is included in the reduced filtered TD-CRI.

22. The system of claim 14, wherein the at least one processor is further configured with instructions for:
obtaining, by the sensing algorithm manager, the reduced CRI;
generating, by the sensing algorithm manager, a reconstructed CSI based on the reduced CRI; and
executing, by the sensing algorithm manager, a sensing algorithm according to the reconstructed CSI to obtain a sensing result.

23. The system of claim 18, further comprising:
obtaining, by the sensing algorithm manager, the reduced filtered TD-CRI, the location information, and the frequency domain bit map;
generating, by the sensing algorithm manager, a reconstructed TD-CRI based on the location information, the frequency domain bit map, and the principal impulses of the filtered TD-CRI;
generating, by the sensing algorithm manager, a reconstructed CSI according to the reconstructed TD-CRI; and
executing, by the sensing algorithm manager, a sensing algorithm according to the reconstructed CSI to obtain a sensing result.

24-26. (canceled)
